(19) United States
(12) Patent Application Publication
(10) Pub. No.: US 2015/0203924 A1
(43) Pub. Date: Jul. 23, 2015

(54) DIAGNOSTIC TOOLS FOR HERBICIDE RESISTANCE IN PLANTS

(71) Applicant: PURDUE RESEARCH FOUNDATION, West Lafayette, IN (US)

(72) Inventors: Burkhard Schulz, Lafayette, IN (US); Stephen Craig Weller, West Lafayette, IN (US); Michael R Gribskov, West Lafayette, IN (US); Karthik Ramaswamy Padmanabhan, West Lafayette, IN (US); Kabelo Segboye, Mochudi (BW)

(73) Assignee: PURDUE RESEARCH FOUNDATION, West Lafayette, IN (US)

(21) Appl. No.: 14/557,580

(22) Filed: Dec. 2, 2014

Related U.S. Application Data
(60) Provisional application No. 61/910,770, filed on Dec. 2, 2013.

(51) Int. Cl. C12Q 1/68

(52) U.S. Cl. CPC C12Q 1/6895; C12Q 2600/13; C12Q 2600/158; C12Q 2600/16

(57) ABSTRACT

Embodiments of the present disclosure relate generally to kits for identifying herbicide resistant plants and methods for determining whether a plant is herbicide resistant. The methods are based on the discovery that some genes are over-expressed or under-expressed in herbicide resistant plants when compared to herbicide-sensitive plants prior to application of the herbicide. The methods include determining the expression level of the one or more genes of interest in a biological sample, such as via qRT PCR, and determining whether the biological sample is from an herbicide-resistant plant based on the expression level. In an embodiment, the kit and methods are used to identify glyphosate-resistant Ambrosia trifida. The kits and methods can be used to improve crop management strategies.

### CROSS-REFERENCE TO RELATED APPLICATIONS

The present U.S. patent application is related to and claims the priority benefit of U.S. Provisional Patent Application Ser. No. 61/910,770, filed Dec. 2, 2013, the contents of which is hereby incorporated by reference in its entirety into this disclosure.

### TECHNICAL FIELD

This disclosure relates to kits and methods can be used to improve crop management strategies, and in particular to kits and methods for identifying herbicide resistant plants.

### BACKGROUND

This section introduces aspects that may help facilitate a better understanding of the disclosure. Accordingly, these statements are to be read in this light and are not to be understood as admissions about what is or is not prior art.

Giant ragweed (Ambrosia trifida L.) is one of the most damaging weeds in the Corn Belt and the southern United States and in some parts of Canada (Abul-Fatih 1979; Bassett and Crompton, 1982; Webster et al., 1994). Prior to GMO glyphosate-resistant crops, giant ragweed was the most troublesome weed in Midwestern crops and its management has become even more difficult with the recent occurrence of glyphosate-resistant giant ragweed.

Giant ragweed has a large seed ranging from 27 to 45 mg in weight. The seed has a high energy reserve which is beneficial in allowing seedlings to emerge and grow quickly in the spring (Hartzler, et al., 2002). Giant ragweed emerges in early spring with germination highest between March and May but germination can occur through mid-July (Abul-Fatih and Bazzaz, 1979). Rapid emergence and growth of giant ragweed seedlings make it very competitive with crops and allows it to dominate any cropping system if left uncontrolled. Giant ragweed is more competitive in soybean than in corn and season long interference from one Giant ragweed plant per m² in soybean resulted in a yield loss of over 75% compared to weed free soybean (Baysinger and Sims, 1991). One plant per 10 m² in corn fields reduced yield by 10% (Baysinger and Sims, 1991). Giant ragweed has successfully adapted to thrive in both conventionally tilled and no-till crop production systems.

The widespread adoption of glyphosate-resistant cropping systems occurred first in soybean (Glycine max L.) in 1996 followed by canola (Brassica campestris L.), cotton (Gossypium hirsutum L.) and corn (Zea mays L.) and the use of glyphosate significantly increased in these systems. Since the late 1990's, glyphosate-resistant crops have become dominate because glyphosate offers excellent weed control and crop safety. The system is simple and its use has reduced cost of weed control and fuel costs and improved soil conservation through widespread adoption of no-till soil and crop management (Feng et al., 2010).

Glyphosate-resistant crops have led to changes in herbicide use patterns with glyphosate being often the only herbicide used for weed control (Reddy and Norsworthy, 2010). In glyphosate-resistant cropping systems, glyphosate is often applied before planting (preplant) as a weed “burndown treatment” or preemergence and post emergence. Glyphosate is applied at least one of these times but is often applied multiple times, which has led to an overreliance and repeated use for weed control in glyphosate-resistant crops. These use patterns resulted in strong selection pressure for the evolution of glyphosate-resistance in weeds (Duke and Powles, 2009).

Glyphosate was introduced commercially as a non-selective total kill herbicide in 1974 and was used worldwide for more than 20 years with no reports of glyphosate-resistance in weeds species (Bradshaw et al., 1997; Cereira and Duke, 2006). Glyphosate-resistance was first reported in rigid grass (Lolium rigidum) from an orchard in Australia and soon thereafter in goosegrass (Eleusine indica) in Malaysia (Duke and Powles 2009; Lee and Ngim, 2000). However, since 1996, with the introduction of glyphosate-resistant crops, twenty-four weed species in seventeen countries, including Brazil, Canada, Australia, and the United States have developed resistance to glyphosate. This resistance is especially prevalent in Amaranthus, Ambrosia, Conyza, and Lolium species (Heap, 2012). Glyphosate-resistance in a glyphosate resistant cropping system was first reported in Canada fleabane (Conyza canadensis L.) in the state of Delaware in 2001 (VanGessel, 2001). There are now thirty-three states in the USA that have reported glyphosate-resistance in fourteen weed species.

The database website of the International Survey of Herbicide Resistant Weeds reported that populations of glyphosate-resistant giant ragweed are now present in eleven US states. The existence of glyphosate-resistant giant ragweed and many other problematic weeds in glyphosate-tolerant cropping systems threatens the utility of glyphosate and the glyphosate-tolerant-cropping systems due to poor control of resistant weeds in agronomic crops resulting in large yield losses. The increasing prevalence of glyphosate-resistant giant ragweed and resistance to some acetolactate synthase inhibitors (ALS) will result in increased use of alternative herbicides, an increase in tilling and a possible return to less environmentally beneficial practices.

The main resistance mechanisms initially identified in plant species that survived glyphosate treatment included mutations of the 5-enol-pyruvylshikimate-phosphate synthase (EPSPS) gene (target-site resistance) and reduced translocation (non-target site-based resistance), (Feng et al., 2004; Powles and Preston, 2006), amplification of the EPSPS gene, vacuolar sequestration of glyphosate (Ge et al., 2010) and catabolic metabolism of glyphosate (Reddy et al., 2008). Mutations in the EPSPS gene result in amino acid changes involving replacing a proline at position 106 with a serine (P106S), alanine (P106A), or threonine (P106T), which interfere with glyphosate binding to the target site (Baerson et al., 2002; Wakelin and Preston, 2006a; Powles and Preston, 2006). Glyphosate-resistant populations of Eleusine indica from Malaysia were shown to contain two mutations (P106S and P106T) (Baerson et al., 2002). A P106T and P106A substitutions were found in Glyphosate-resistant Lolium rigidum from Australia and South Africa, respectively (Wakelin and Preston, 2006a). Glyphosate-resistant weeds with non-target site mechanism can be caused by altered translocation of glyphosate to the susceptible, actively growing tissues (Shaner, 2009). A nuclear encoded gene with partial or complete dominance causes this form of resistance (Lorraine-Colwill et al., 2001; Wakelin and Preston, 2006b; Preston et al., 2009). Resistant rigid ryegrass with altered translocation accumulated about 50% of the applied glyphosate into the leaf tips, whereas susceptible plants accumulated the majority of glyphosate in the roots and shoot meristem (Lorraine-Colwill et al., 2002). Feng et al. (2004) observed reduced translocation from leaves to root tissues in resistant horseweed (Conyza canadensis) biotypes, and this was directly related to the sequestration of the majority of applied glyphosate in the vacuoles of mature leaves in resistant plants within 24 h of application (Ge et al., 2010). It is speculated that a tonoplast localized glyphosate transporter is either only present or is up-regulated in resistant biotypes; however, the detailed mechanism and the transporter involved have not yet been described (Yuan et al., 2007; Shaner, 2009). Amplification of the EPSPS gene in Glyphosate-resistant Palmer amaranth (Amaranthus palmeri S.) was shown to confer a high level of glyphosate resistance and some plants in the Palmer amaranth population had a greater than 100-fold increase in EPSPS copies (Gaines et al., 2013).

In the case of giant ragweed, glyphosate-resistant biotypes were first identified in Ohio in 2004 and now have been reported in ten other US states and Ontario, Canada (Heap, 2012). Westhoven et al. (2008) confirmed glyphosate-resistant giant ragweed in Noble County Indiana in 2004 and surveys in Indiana between 2005 and 2006 indicated that 22 out of 101 randomly surveyed fields contained some glyphosate-resistant giant ragweed plants. The specific mechanism of resistance in giant ragweed has not been analyzed.

The identification of herbicide resistance is becoming increasingly important in view of discussions on increasing use of herbicides, ineffectiveness of herbicides, and efficient use of one or more strategies to increase yield. Ideally, such identification method is both quick and simple, without the need for an extensive laboratory set-up. Furthermore, the method should provide results that allow identification of herbicide resistance without expert interpretation, but which hold up under expert scrutiny if necessary. An effective identification tool would assist in weed control strategy. Specific tools for use in the identification of herbicide resistance in biological samples are described herein.

### SUMMARY

Embodiments of the present disclosure relate generally to methods of identifying herbicide-resistant plants, kits for implementing the method, and primers used in the kits. In an embodiment, a method of identifying herbicide resistant plants including screening a plant for an enhanced expression level of one or more genes is provided. In an embodiment, a kit comprising primers for amplifying genes associated with herbicide resistance is provided. The kit and method are directed to identifying one or more of a set of differentially expressed genes that are down-regulated or up-regulated in glyphosate-resistant plants in comparison to glyphosate-sensitive plants without prior treatment with glyphosate herbicides.

In a first aspect, a method for identifying an herbicide-resistant plant is provided. In some embodiments, the method includes determining one or more genes that are differentially expressed in an herbicide-resistant plant compared to an herbicide-sensitive plant of the same species; amplifying a nucleic acid associated with the one or more genes present in a biological sample from a plant; quantifying expression of the nucleic acid; and determining that the plant is herbicide-resistant based on the quantification of the nucleic acid.

In some embodiments, the biological sample is from Ambrosia trifida. In some embodiments, the herbicide is glyphosate (IUPAC name: N-(phosphonomethyl)glycine, UNII number 4632WW1X5A). In further embodiments, the expression of the nucleic acid is quantified using qRT PCR. In still further embodiments, the one or more genes have at least four-fold differential expression in the herbicide-resistant plant compared to the herbicide sensitive plant prior to application of the herbicide.

In an embodiment, the one or more genes is associated with cDNA selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 48, SEQ ID NO: 49, SEQ ID NO: 50, SEQ ID NO: 51, SEQ ID NO: 52, SEQ ID NO: 53, and SEQ ID NO: 54.

In a second aspect, a related method for identifying an herbicide-resistant plant is provided. In some embodiments, the method includes amplifying a nucleic acid sequence present in a biological sample from a plant using at least two primers, wherein said at least two primers recognize a gene that is differentially expressed in an herbicide-resistant plant compared to an herbicide-sensitive plant of the same species; quantifying expression of the nucleic acid sequence in the biological sample; and determining that the plant is herbicide resistant based on the quantification of the nucleic acid.

In some embodiments, the at least two primers are selected from the group consisting of: SEQ ID NO: 121 and SEQ ID NO: 122, SEQ ID NO: 123 and SEQ ID NO: 124, SEQ ID NO: 125 and SEQ ID NO: 126, SEQ ID NO: 127 and SEQ ID NO: 128, SEQ ID NO: 129 and SEQ ID NO: 130, SEQ ID NO: 131 and SEQ ID NO: 132, SEQ ID NO: 133 and SEQ ID NO: 134, SEQ ID NO: 135 and SEQ ID NO: 136, SEQ ID NO: 137 and SEQ ID NO: 138, SEQ ID NO: 139 and SEQ ID NO: 140, SEQ ID NO: 141 and SEQ ID NO: 142, SEQ ID NO: 143 and SEQ ID NO: 144, SEQ ID NO: 145 and SEQ ID NO: 146, SEQ ID NO: 147 and SEQ ID NO: 148, SEQ ID NO: 149 and SEQ ID NO: 150, SEQ ID NO: 151 and SEQ ID NO: 152, SEQ ID NO: 153 and SEQ ID NO: 154, SEQ ID NO: 155 and SEQ ID NO: 156, SEQ ID NO: 157 and SEQ ID NO: 158, SEQ ID NO: 159 and SEQ ID NO: 160, SEQ ID NO: 161 and SEQ ID NO: 162, SEQ ID NO: 163 and SEQ ID NO: 164, SEQ ID NO: 165 and SEQ ID NO: 166, and SEQ ID NO: 167 and SEQ ID NO: 168.

In further embodiments, the at least two primers are selected from the group consisting of: SEQ ID NO: 169 and SEQ ID NO: 170, SEQ ID NO: 171 and SEQ ID NO: 172, SEQ ID NO: 173 and SEQ ID NO: 174, SEQ ID NO: 175 and SEQ ID NO: 176, SEQ ID NO: 177 and SEQ ID NO: 178, SEQ ID NO: 179 and SEQ ID NO: 180, SEQ ID NO: 181 and SEQ ID NO: 182, SEQ ID NO: 183 and SEQ ID NO: 184, SEQ ID NO: 185 and SEQ ID NO: 186, SEQ ID NO: 187 and SEQ ID NO: 188, SEQ ID NO: 189 and SEQ ID NO: 190, SEQ ID NO: 191 and SEQ ID NO: 192, SEQ ID NO: 193 and SEQ ID NO: 194, SEQ ID NO: 195 and SEQ ID NO: 196, SEQ ID NO: 197 and SEQ ID NO: 198, SEQ ID NO: 199 and SEQ ID NO: 200, SEQ ID NO: 201 and SEQ ID NO: 202, SEQ ID NO: 203 and SEQ ID NO: 204, SEQ ID NO: 205 and SEQ ID NO: 206, SEQ ID NO: 207 and SEQ ID NO: 208, and SEQ ID NO: 209 and SEQ ID NO: 210.

In additional embodiments, the biological sample is from Ambrosia trifida and the herbicide is glyphosate.

In a further aspect, a kit for identifying an herbicide-resistant plant is provided. In some embodiments, the kit includes at least two primers, wherein said at least two primers recognize a gene that is differentially expressed in an herbicide-resistant plant compared to an herbicide-sensitive plant of the same species.

In an embodiment, the kit includes at least one of a positive control and a negative control. In some embodiments, the gene is associated with cDNA selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 48, SEQ ID NO: 49, SEQ ID NO: 50, SEQ ID NO: 51, SEQ ID NO: 52, SEQ ID NO: 53, and SEQ ID NO: 54. In further embodiments, the kit includes components of a qRT PCR solution for amplifying a sequence associated with the gene. In some embodiments, the plant is Ambrosia trifida and the herbicide is glyphosate.

In a still further aspect, a specific probe for the identification of an herbicide-resistant plant is provided. In some embodiments, the specific probe includes a nucleic acid that recognizes a part of a sequence of a gene of interest, wherein the gene of interest is differentially expressed in an herbicide-resistant plant compared to an herbicide-sensitive plant of the same species; and a detectable marker linked to the nucleic acid.

In some embodiments, the detectable marker is selected from the group consisting of a radioactive marker and a fluorescent marker.

In an aspect, a primer pair comprising a first primer capable of recognizing a first portion of a gene of interest and a second primer capable of recognizing a second portion of a gene of interest, wherein the gene of interest is differentially expressed in an herbicide-resistant plant compared to an herbicide-sensitive plant of the same species.

In some embodiments, the primer pair is selected from the group consisting of: SEQ ID NO: 121 and SEQ ID NO: 122, SEQ ID NO: 123 and SEQ ID NO: 124, SEQ ID NO: 125 and SEQ ID NO: 126, SEQ ID NO: 127 and SEQ ID NO: 128, SEQ ID NO: 129 and SEQ ID NO: 130, SEQ ID NO: 131 and SEQ ID NO: 132, SEQ ID NO: 133 and SEQ ID NO: 134, SEQ ID NO: 135 and SEQ ID NO: 136, SEQ ID NO: 137 and SEQ ID NO: 138, SEQ ID NO: 139 and SEQ ID NO: 140, SEQ ID NO: 141 and SEQ ID NO: 142, SEQ ID NO: 143 and SEQ ID NO: 144, SEQ ID NO: 145 and SEQ ID NO: 146, SEQ ID NO: 147 and SEQ ID NO: 148, SEQ ID NO: 149 and SEQ ID NO: 150, SEQ ID NO: 151 and SEQ ID NO: 152, SEQ ID NO: 153 and SEQ ID NO: 154, SEQ ID NO: 155 and SEQ ID NO: 156, SEQ ID NO: 157 and SEQ ID NO: 158, SEQ ID NO: 159 and SEQ ID NO: 160, SEQ ID NO: 161 and SEQ ID NO: 162, SEQ ID NO: 163 and SEQ ID NO: 164, SEQ ID NO: 165 and SEQ ID NO: 166, and SEQ ID NO: 167 and SEQ ID NO: 168.

In further embodiments, the primer pair is selected from the group consisting of: SEQ ID NO: 169 and SEQ ID NO: 170, SEQ ID NO: 171 and SEQ ID NO: 172, SEQ ID NO: 173 and SEQ ID NO: 174, SEQ ID NO: 175 and SEQ ID NO: 176, SEQ ID NO: 177 and SEQ ID NO: 178, SEQ ID NO: 179 and SEQ ID NO: 180, SEQ ID NO: 181 and SEQ ID NO: 182, SEQ ID NO: 183 and SEQ ID NO: 184, SEQ ID NO: 185 and SEQ ID NO: 186, SEQ ID NO: 187 and SEQ ID NO: 188, SEQ ID NO: 189 and SEQ ID NO: 190, SEQ ID NO: 191 and SEQ ID NO: 192, SEQ ID NO: 193 and SEQ ID NO: 194, SEQ ID NO: 195 and SEQ ID NO: 196, SEQ ID NO: 197 and SEQ ID NO: 198, SEQ ID NO: 199 and SEQ ID NO: 200, SEQ ID NO: 201 and SEQ ID NO: 202, SEQ ID NO: 203 and SEQ ID NO: 204, SEQ ID NO: 205 and SEQ ID NO: 206, SEQ ID NO: 207 and SEQ ID NO: 208, and SEQ ID NO: 209 and SEQ ID NO: 210.

Other aspects and features, as recited by the claims, will become apparent to those skilled in the art upon review of the following non-limited detailed description in conjunction with the accompanying figures.

### BRIEF DESCRIPTION OF DRAWINGS

Having thus described embodiments in general terms, reference will now be made to the accompanying drawings, which are not necessarily drawn to scale, and wherein:

FIGS. 1a-1c provide a comparison of resistant and sensitive Giant ragweed populations at varying rates of glyphosate. In FIG. 1a, a whole plant tissue was harvested at 21 days after treatment and fresh weight was measured. In FIG. 1b, the table provides data on a comparison within populations (resistant or sensitive). In FIG. 1c, representative photographs of phenotypic presentation of control, glyphosate-sensitive and glyphosate-resistant plants after varying concentrations of glyphosate twenty-one days after treatment are provided, in accordance with embodiments of the disclosure;

FIGS. 2a and 2b provide representative photographs (FIG. 2a) of sensitive and resistant biotypes 24 hours and 3 weeks after treatment as well as pie charts (FIG. 2b) depicting a visual rating of the sensitive and resistant biotypes, in accordance with embodiments of the disclosure;

FIG. 3 provides data on chlorophyll fluorescence measurements in resistant and sensitive plants at 0 hrs, 6 hrs, and 12 hrs after treatment with 1× field rate glyphosate, in accordance with an embodiment of the disclosure;

FIGS. 4a and 4b provide representative photographs of localized application of glyphosate on resistant and sensitive plants, in accordance with an embodiment of the disclosure;

FIG. 5 provides data on translocation of glyphosate in resistant and sensitive plants at different concentrations at different times post-treatment, in accordance with an embodiment of the disclosure;

FIGS. 6a and 6b provide data on salicylic acid (FIG. 6a) and jasmonic acid (FIG. 6b) production in resistant and sensitive plants, in accordance with an embodiment of the disclosure;

FIG. 7 provides the relative seed numbers produced by glyphosate-resistant and sensitive biotypes grown at total input density of 4 plants 0.25 m², in accordance with an embodiment of the disclosure;

FIG. 8 provides the relative shoot dry biomass of glyphosate-resistant and glyphosate susceptible giant ragweed biotypes grown at total input density of 4 plants 0.25 m², in accordance with an embodiment of the disclosure;

FIG. 9 provides the results of a study comparing H₂O₂ accumulation on glyphosate treated mature and immature glyphosate-resistant and glyphosate-sensitive leaves using DAB staining, in accordance with an embodiment of the disclosure; and

FIG. 10 provides the enrichment of gene ontology (GO)-terms in the biological process category, in accordance with an embodiment of the disclosure.

### DETAILED DESCRIPTION

Embodiments of the present disclosure will now be described more fully hereinafter with reference to the accompanying drawings, in which some, but not all, embodiments are shown. Indeed, the disclosure may be embodied in many different forms and should not be construed as limited to the embodiments set forth herein; rather, these embodiments are provided so that this disclosure will satisfy applicable legal requirements. Additionally, while embodiments are disclosed as “comprising” elements, it should be understood that the embodiments may also “consist of” elements or “consist essentially of” elements. Where possible, any terms expressed in the singular form herein are meant to also include the plural form and vice versa unless explicitly stated otherwise. Also, as used herein, the term “a” and/or “an” shall mean “one or more,” even though the phrase “one or more” is also used herein. Like numbers refer to like elements throughout.

Throughout this disclosure, various information sources are referred to and/or are specifically incorporated. The information sources include scientific journal articles, patent documents, textbooks, and websites. While the reference to these information sources clearly indicates that they can be used by one of skill in the art, each and every one of the information sources cited herein are specifically incorporated in their entirety, whether or not a specific mention of “incorporation by reference” is noted. The contents and teachings of each and every one of the information sources can be relied on and used to make and use embodiments of the disclosure.

The present disclosure is based on newly discovered differences between herbicide-resistant plants and herbicide-sensitive plants. In an embodiment, a method evaluates the expression level of one or more genes in Ambrosia trifida L. (Giant ragweed) to determine whether the A. trifida is glyphosate-resistant or glyphosate-sensitive. Unexpectedly, one or more genes are overexpressed in glyphosate-resistant A. trifida compared to glyphosate-sensitive A. trifida. Similarly, one or more genes are underexpressed in glyphosate-resistant A. trifida compared to glyphosate-sensitive A. trifida.

While the examples disclosed herein are directed to A. trifida, one skilled in the art would understand that the method for detecting herbicide-resistance may be applied to other species of plants without undue experimentation. For example, the method for detecting herbicide-resistance may be applied to all species in the genus Ambrosia. In another example, based on the principle that phylogenetically related species have similar physiological conditions and responses, the method for detecting herbicide-resistance may be applied to all genera in the Asteraceae. In other embodiments, the method is used to detect herbicide-resistance in flowering plants in the class Magnoliopsida, in seed plants in the division Magnoliophyta, or in vascular plants in general.

The examples are directed to identifying resistance to the herbicide glyphosate but one skilled in the art would understand that the mechanism by which the resistance developed may also provide resistance to other herbicides. For example, plants that have an herbicide resistance mechanism to glyphosate may have also developed an herbicide resistance mechanism to herbicides such as paraquat, mesotrione, glufosinate, acetochlor, 2,4-D, mesosulfuron, Smetolachlor, fenoxaprop, clodinafop, nicosulfuron, and/or atrazine. As used herein, “herbicide-resistant” means that a plant has an increased tolerance to herbicide application. The increased tolerance may mean that the plant survives application of the herbicide at usual field rates. In an embodiment, the increased tolerance means that the plant initially indicates senescence and rapid cell death of one or more portions of the plant but that the plant survives based on meristematic growth. In some embodiments, the increased tolerance is limited to specific concentrations of the herbicide, specific times of applications of herbicide, specific type of application, or specific frequency of application of the herbicide. In some embodiments, the increased tolerance results in a delay in senescence of the entire plant from the administration of the herbicide. In some embodiments, the increased tolerance is compared to a wild type or control plant. Glyphosate is a specific type of herbicide to which plants may be resistant.

As used herein, “herbicide-sensitive” means that a plant has an expected response to application of the herbicide. Thus, an herbicide that is applied with the intention that it will kill a specific type of plant will be effective in managing the plant if the plant is herbicide sensitive. In other words, an herbicide-sensitive plant has the expected response to application of the herbicide. In some embodiments, the herbicide-sensitive plant is a wild type or nontransformed plant. For example, the herbicide-sensitive plant may be a naturally occurring biotype of a plant. In some embodiments, plants are devised to be herbicide-sensitive. Glyphosate is a specific type of herbicide to which plants may be sensitive.

Herbicide application may be at any life stage of the plant, including seeds, seedlings, and flowering. Herbicide may be applied directly to the plant, to the seed, to the soil, through the water, via a mist, or the like. In some embodiments, herbicide application method does not affect whether the plant is resistant to the herbicide.

The present disclosure is directed to providing a method for identifying an herbicide-resistant plant, or cells or tissues thereof. In an embodiment, the method is based on identifying the expression level of one or more genes in the plant. In some embodiments, the method includes using primers or probes which specifically recognize a portion of the sequence of the gene or genes of interest. In some embodiments, a PCR-based technique is used to quantify the expression of one or more genes that are differentially expressed in resistant plants compared to sensitive plants prior to treatment. In other words, basal expression levels are heightened or lowered in resistant plants compared to sensitive plants prior to herbicide treatment.

More specifically, the disclosure relates to a method comprising amplifying a sequence of a nucleic acid present in biological samplings using a polymerase chain reaction with at least two primers that recognize one or more of the genes of interest.

In some embodiments, the identification is performed using polymerase chain reaction. The method may also include providing a detectable marker specific to the one or more genes or interest. In embodiments, the detection is performed using an Enzyme-Linked Immunosorbent Assay (ELISA), a quantitative real-time polymerase chain reaction (qPCR), or a RNA-hybridization technique.

The disclosure further relates to kits for identifying herbicide-resistant plants, said kits comprising at least two primers or probes that specifically recognize one or more of the genes of interest. For example, SEQ ID NO: 1 discloses cDNA associated with a gene that is expressed at a ratio of 26:1 in glyphosate-resistant plants compared to glyphosate-sensitive plants prior to treatment with glyphosate. Primers have been developed to amplify and/or quantify the expression of the gene associated with SEQ ID NO: 1. By evaluating the expression level of one or more genes of interest, one skilled in the art is able to determine whether a plant sample comes from a glyphosate-resistant plant. In an embodiment, the kit includes more than one primer pair for different genes of interest. For example, primers for a gene that is over-expressed in a resistant plant as well as a gene that is under-expressed in a resistant plant compared to a sensitive plant may be included in the kit. The kit may also include one or more positive or negative controls.

In some embodiments, the kits includes a specific probe having a sequence which corresponds to or is complementary to a sequence having between 80% and 100% sequence identity with a specific region of the one or more genes of interest. In some embodiments, the kit includes a specific probe which corresponds to or is complementary to a sequence having between 90% and 100% sequence identity with a specific region of the one or more genes of interest.

According to another embodiment of the disclosure, primers sequences are disclosed comprising the corresponding or complementary DNA to the portions of the genes of interest that are amplified in order to determine over-expression or under-expression. Additionally, cDNA sequences corresponding to the one or more genes of interest are provided. While exemplary nucleotide sequences will be provided herein, it should be understood that additional nucleotide sequences may be diagnostic for herbicide-resistance due to the degeneracy of the DNA code.

The methods, kits, and primers of the disclosure can be used for different purposes including, but not limited to the following: identifying the presence or absence of herbicide resistance in plants, plant material such as seeds or cuttings; determining the presence of herbicide-resistant weeds in crop fields; and tailoring an herbicide regime to effectively and economically manage weeds affecting agricultural crops.

### DEFINITIONS

The following terms are used throughout this disclosure:

The term “kit” as used herein refers to a set of reagents for the purpose of performing the method disclosed herein, more particularly, the identification of herbicide resistant plants based on biological samples. More particularly, an embodiment of the kit comprises at least one or two specific primers, as described above for identification of the one or more genes of interest. Optionally, the kit can further comprise any other reagent included in a PCR identification protocol. Alternatively or additionally, according to another embodiment, the kit comprises a specific probe, which specifically hybridizes with nucleic acid of biological samples to identify the presence of the one or more genes of interest therein. Optionally, the kit can further comprise any other reagent (such as but not limited to hybridizing buffer, labeling chemicals, signal intensifier, etc.) for identification of the one or more genes of interest in biological samples, using the specific probe.

The term “gene” as used herein refers to the partial or complete coding sequence of a gene, its complement, and its 5′ or 3′ untranslated regions. A gene is also a functional unit of inheritance and in physical terms is a particular segment or sequence of nucleotides along a molecule of DNA (or RNA, in the case of RNA viruses) involved in producing a polypeptide chain. The latter may be subjected to subsequent processing such as chemical modification or folding to obtain a functional protein or polypeptide. A gene may be isolated, partially isolated, or found with an organism's genome. By way of example, a transcription factor gene encodes a transcription factor polypeptide, which may be functional or require processing to function as an initiator of transcription.

The term “operably linked” or “operably-linked” as used herein refers to positioning of a regulatory region and a nucleotide sequence to enable influencing transcription initiation or translation initiation or transcription termination of the nucleotide sequence.

The term “over-expression” as used herein refers to an increased expression level of a gene in a plant, plant cell, or plant tissue. In an embodiment over-expression is measured compared to expression of a wild-type plant, cell, or tissue, at any developmental or temporal stage. In another embodiment, over-expression is measured compared to an absolute level of expression. Over-expression can occur when, for example, the genes encoding one or more polypeptides are under the control of a strong promoter (e.g., the cauliflower mosaic virus 35S transcription initiation region). Over-expression may also be under the control of an inducible, stress-responsive, or tissue-specific promoter. Over-expression may occur throughout the plant, in specific tissue of the plant, or in the presence or absence of particular environmental signals, depending on the promoter used. Over-expression may take place in plant cells normally lacking expression of polypeptides functionally equivalent or identical to the present polypeptides. Over-expression may also occur in plant cells where endogenous expression of the present polypeptide or functionally equivalent molecules normally occurs, but such normal expression is at a lower level. Over-expression results in a greater than normal production, or “overproduction” of the polypeptide in the plant, cell, or tissue.

The term “plant” as used herein includes whole plants, shoot vegetative organs/structures (for example, leaves, stems, and tubers), roots, flowers and floral organs/structures (for example, bracts, sepals, petals, stamens, carpels, anthers, and ovules), seed (including embryo, endosperm, and seed coat), and fruit (the mature ovary), plant tissue (for example, vascular tissue, ground tissue, and the like), and cells and cell cultures (for example, guard cells, egg cells, and the like), and progeny of the same.

The term “polynucleotide” as used herein is a nucleic acid molecule comprising a plurality of polymerized nucleotides, e.g., at least about five consecutive polymerized nucleotides. A polynucleotide may be a nucleic acid, oligonucleotide, nucleotide, or any fragment thereof. In many instances, a polynucleotide comprises a nucleotide sequence encoding a polypeptide (or protein) or a domain or fragment thereof. Additionally, the polynucleotide may comprise a promoter, an intron, an enhancer region, a polyadenylation site, a translation initiation site, 5′ or 3′ untranslated regions, a reporter gene, a selectable marker, or the like. The polynucleotide can be single-stranded or double-stranded DNA or RNA. The polynucleotide optionally comprises modified bases or a modified backbone. The polynucleotide can be, e.g., genomic DNA or RNA, a transcript (such as an mRNA), a cDNA, a PCR product, a cloned DNA, a synthetic DNA or RNA, or the like. The polynucleotide can be combined with carbohydrate, lipids, protein, or other materials to perform a particular activity such as transformation or form a useful composition such as a peptide nucleic acid (PNA). The polynucleotide can comprise a sequence in either sense or antisense orientations. “Oligonucleotide” is substantially equivalent to the terms amplimer, amplicon, primer, oligomer, element, target, and probe and in some embodiments is single-stranded.

The term “polypeptide” as used herein is an amino acid sequence comprising a plurality of consecutive polymerized amino acid residues e.g., at least about five consecutive polymerized amino acid residues. In many instances, a polypeptide comprises a polymerized amino acid residue sequence that is a transcription factor or a domain or portion or fragment thereof. Additionally, the polypeptide may comprise: (i) a localization domain; (ii) an activation domain; (iii) a repression domain; (iv) an oligomerization domain; (v) a protein-protein interaction domain; (vi) a DNA-binding domain; or the like. The polypeptide optionally comprises modified amino acid residues, naturally occurring amino acid residues not encoded by a codon, and/or non-naturally occurring amino acid residues.

The term “primer” as used herein encompasses any nucleic acid that is capable of priming the synthesis of a nascent nucleic acid in a template-dependent process, such as PCR. Typically, primers are oligonucleotides from 10 to 30 nucleotides in length, but longer sequences may be used. Primers may be provided in single or double-stranded form. Probes may be used as primers, but are designed to bind to the target DNA or RNA and need not be used in an amplification process.

The term “protein” as used herein refers to an amino acid sequence, oligopeptide, peptide, polypeptide or portions thereof whether naturally occurring or synthetic.

The term “regulatory region” as used herein refers to nucleotide sequences that, when operably linked to a sequence, influence transcription initiation or translation initiation or transcription termination of said sequence and the rate of said processes, and/or stability, and/or mobility of transcription or translation product. Regulatory regions include, without limitation, promoter sequences, enhancer sequences, response elements, protein recognition sites, inducible elements, protein-binding sequences, 5′ and 3′ untranslated regions (UTRs), transcriptional start sites, termination sequences, polyadenylation sequences, and introns. Regulatory regions can be classified into two categories: promoters and other regulatory regions.

As used herein, “sequence identity” with regard to nucleotide sequences (DNA or RNA), refers to the number of positions with identical nucleotides divided by the number of nucleotides in the shorter of the two sequences. The alignment of the two nucleotide sequences is performed by the Wilbur and Lipmann algorithm (Wilbur and Lipmann, 1983, Proc. Nat. Acad. Sci. USA 80:726) using a window-size of 20 nucleotides, a word length of 4 nucleotides, and a gap penalty of 4. Computer-assisted analysis and interpretation of sequence data, including sequence alignment as described above, can, e.g., be conveniently performed using the sequence analysis software package of the Genetics Computer Group (GCG, University of Wisconsin Biotechnology center). Sequences are indicated as “essentially similar” when such sequences have a sequence identity of at least about 75%, particularly at least about 80%, more particularly at least about 85%, quite particularly about 90%, especially about 95%, more especially about 100%. It is clear that when RNA sequences are said to be essentially similar or have a certain degree of sequence identity with DNA sequences, thymidine (T) in the DNA sequence is considered equal to uracil (U) in the RNA sequence.

The term “under-expression” as used herein refers to a decreased expression level of a gene in a plant, plant cell, or plant tissue. In an embodiment under-expression is measured compared to expression of a wild-type plant, cell, or tissue, at any developmental or temporal stage. In another embodiment, under-expression is measured compared to an absolute level of expression. Under-expression can occur when, for example, the genes encoding one or more polypeptides are inhibited. Under-expression may also be under the control of an inducible, stress-responsive, or tissue-specific promoter. Under-expression may occur throughout the plant, in specific tissue of the plant, or in the presence or absence of particular environmental signals, depending on the promoter used. Under-expression may take place in plant cells normally expressing polypeptides functionally equivalent or identical to the present polypeptides. Underexpression may also occur in plant cells where endogenous expression of the present polypeptide or functionally equivalent molecules normally occurs, but such normal expression is at a higher level. Under-expression results in a less than normal production, or “underproduction” of the polypeptide in the plant, cell, or tissue.

Comparison of Glyphosate-Resistant and Glyphosate-Sensitive A. trifida:
Foliar Response Differences in Resistant and Sensitive A. trifida:

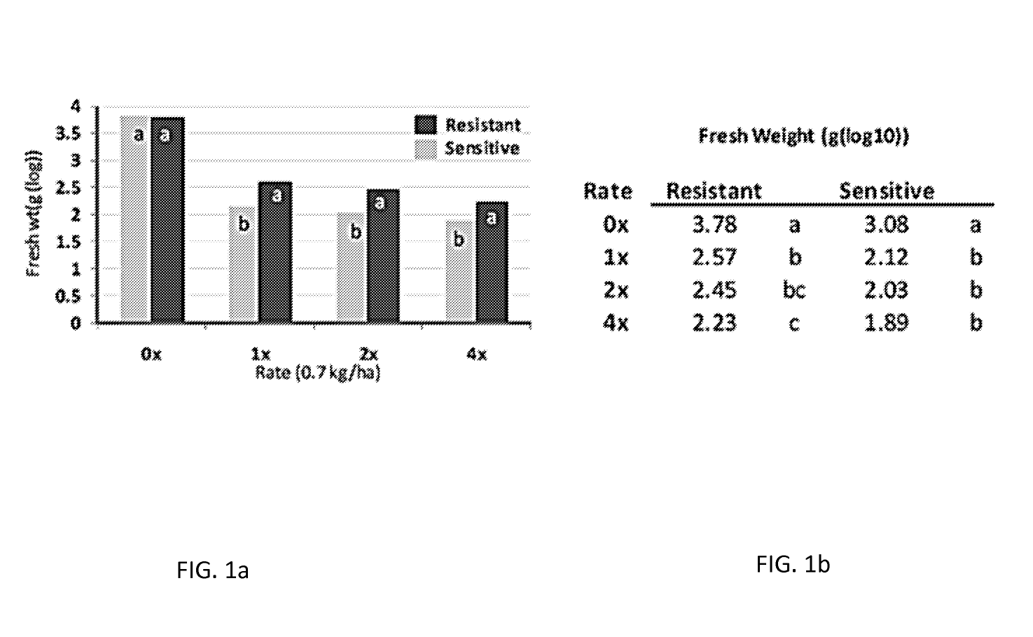

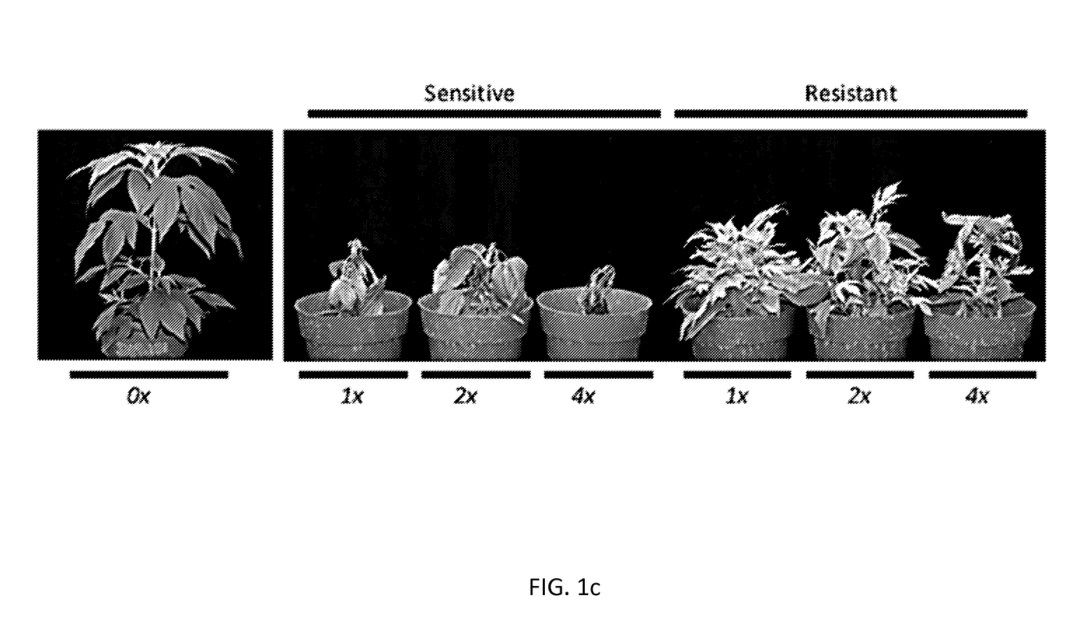

FIGS. 1a-1c provide data relating to glyphosate resistance in a Noble County, IN population of A. trifida. Specifically, FIG. 1a shows that the glyphosate resistance occurred at multiple glyphosate rates (1×=0.7 kg ae ha-1, 2×=1.4 kg ae ha-1, and 4×=2.8 kg ae ha-1). The resistant plants showed increased fresh weight at all treatment levels compared to the sensitive plants. Significance (<0.05) between populations (within rates) is indicated by letters (a, b). In FIG. 1b, the fresh weight of glyphosate-resistant and glyphosate-sensitive plants twenty-one days after treatment is provided. The difference in fresh weight between the resistant and the sensitive plants was significant at 4× treatment. Significance (<0.05) between rates (within populations) is indicated by letters (a, b, c). FIG. 1c provides a visual comparison of plant injury between sensitive and resistant plants at 1×, 2×, and 4× treatment levels after twenty-one days. As shown, the resistant plants exhibited decreased visual injury compared to the sensitive plants.

In this example, Touchdown HiTech® (N-(phosphonomethyl)glycine, in form of the monopotassium salt) (Syngenta Crop Protection, Inc., Greensboro, N.C. 27419) was the glyphosate solution used for plant treatments. This formulation does not contain surfactant but a non-ionic spreader-sticker adjuvant surfactant (NIS) at 0.25% v/v and 1.0% w/v Ammonium Sulfate (AMS) was added. Foliar application of herbicide solution was applied using a compressed-air bench top track sprayer at a pressure of 249 kPa and a volume of 187 L of spray solution ha-1. All unsprayed control plants were treated with the same solution minus glyphosate. All plants were at the third node growth stage at the time of treatment.

Phenotypic Differences Between Resistant and Sensitive A. trifida:

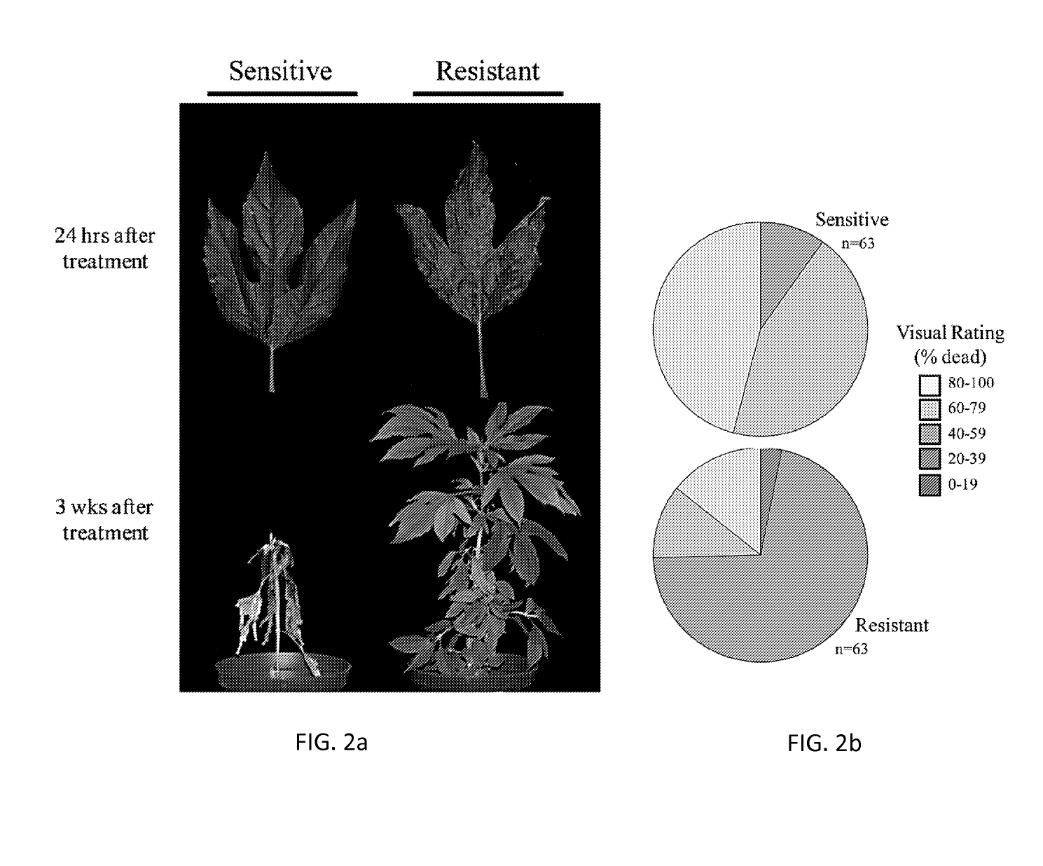

Resistant and sensitive A. trifida exhibited no significant phenotypic differences prior to glyphosate treatment (data not shown). However, after glyphosate treatment a unique phenotype displaying rapid necrosis of mature leaves was observed in glyphosate-resistant plants. FIG. 2 shows resistant and sensitive plant response at 24 hrs and at 3 weeks after treatment with 1× glyphosate field rate. Still referring to FIG. 2, at 24 hrs after treatment, the sensitive and resistant leaves showed similar levels of decline. At three weeks after treatment, however, the sensitive plant showed an extremely decline while the resistant plant recovered from the glyphosate treatment.

FIG. 2 also includes a visual injury rating at 3 weeks after treatment for the sensitive and resistant plants (n=63). The visual injury rating show that resistant plants had a much lower rating of % dead compared to the sensitive plants. The resistant plants exposed to glyphosate expressed significant initial stunting and tissue necrosis of older leaves but were able to resume growth from growing points and meristems (Brabham et al., 2011; Westhoven et al., 2008).

Fluorescence Differences Between Resistant and Sensitive A. trifida:

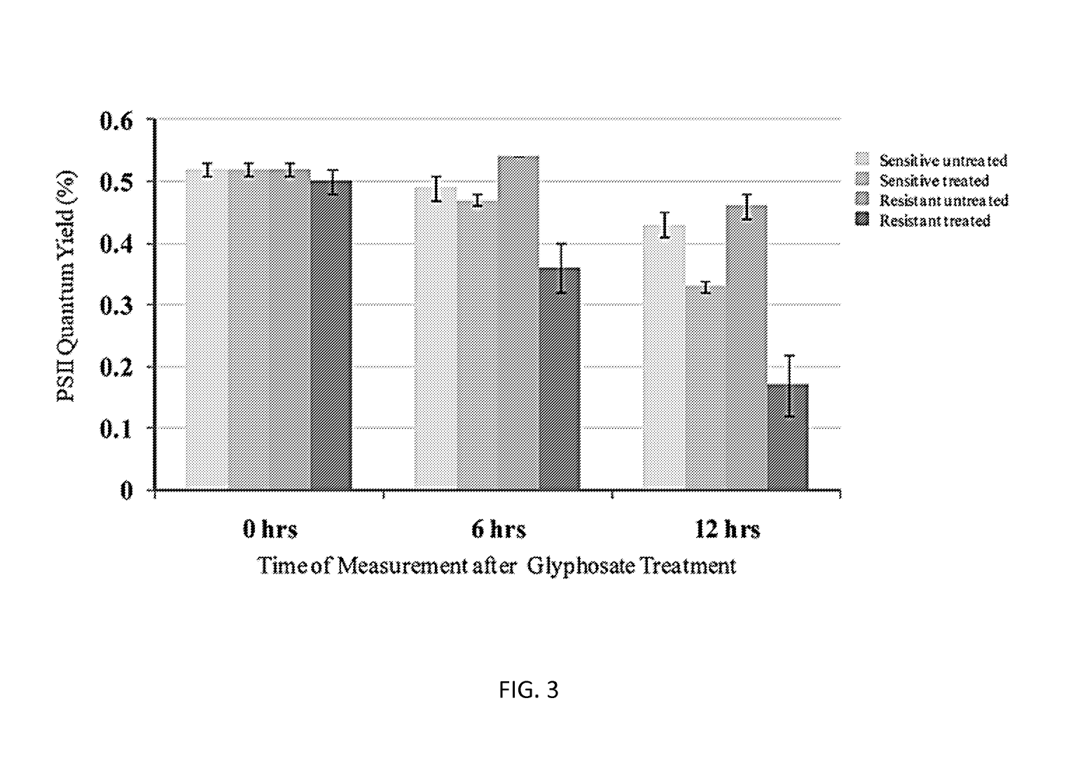

To quantify early physiological reactions related to this resistance mechanism, a method was developed using chlorophyll fluorescence measurements to determine photosynthetic activity. Rapid necrosis in mature leaves occurred in glyphosate-resistant but not in glyphosate-sensitive A. trifida plants within 12 hours of glyphosate treatment, as shown in FIG. 3. FIG. 3 depicts fluorescence measurements at 0, 6, and 12 hours after glyphosate treatment. Treatments at each time point consisted of glyphosate-resistant and glyphosate-sensitive A. trifida untreated and treated leaves.

Additional biochemical experiments, investigating possible mechanisms for glyphosate resistance, showed no difference in EPSPS protein abundance or transcript levels or differences in EPSPS sensitivity to glyphosate challenge between glyphosate-resistant and glyphosate-sensitive plants. DNA sequencing of both alleles of EPSPS in glyphosate-resistant as well as glyphosate-sensitive A. trifida plants revealed no amino acid mutations in the EPSPS enzymes.

In an embodiment, to measure fluorescence the maximum quantum efficiency of photosystem II (Fv/Fm) is determined using an Imaging-PAM fluorometer IMAG-MAX/GS with a mounted LED-Array Illumination Unit IMAG-MAX/L and CCD-Camera (Heinz Walz GmbH, 91090 Effeltrich, Germany) along with ImagingWin software. Briefly, dark acclimated plants are exposed to a short saturating light pulse to maximally reduce the QA pool and where fluorescence is increased to maximum values (Fm) and this is followed by a weak non-actinic modulated measuring light to determine the minimal level of fluorescence (F0) (Oxborough, 2004). Readings are taken at three different time points (0 hr, 6 hr, and 12 hr) after glyphosate treatment and for untreated controls with an initial reading immediately before glyphosate treatment on all plants.

Translocation of Glyphosate in Resistant and Sensitive A. trifida:

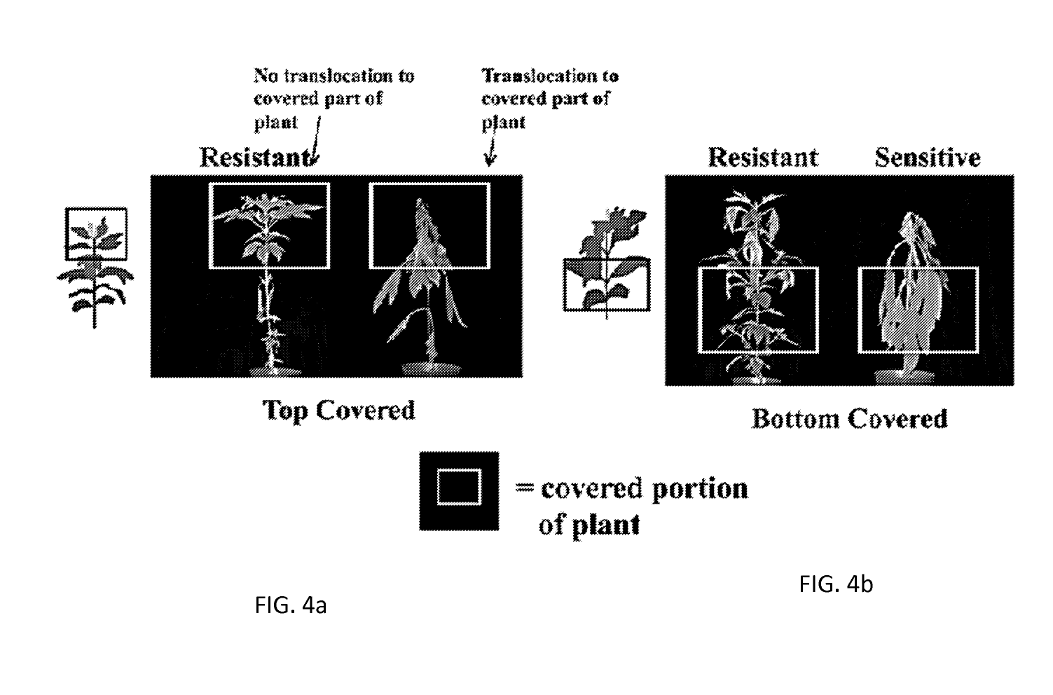

Differential exposure studies to glyphosate treatment showed that covering the upper or lower portion of glyphosate-resistant plants prior to glyphosate application resulted in no translocation of glyphosate away from the treated tissues. However, translocation of glyphosate from the treated tissues occurred in glyphosate-resistant plants. FIGS. 4a and 4b provide the results of tissue specific glyphosate treatments. The white frame represents the portion of the plant covered with foil and not treated with glyphosate. Uncovered portions of plants were sprayed with 0.7 kg ae/ha of glyphosate. Glyphosate-resistant plants did not translocate glyphosate while glyphosate-sensitive plants did.

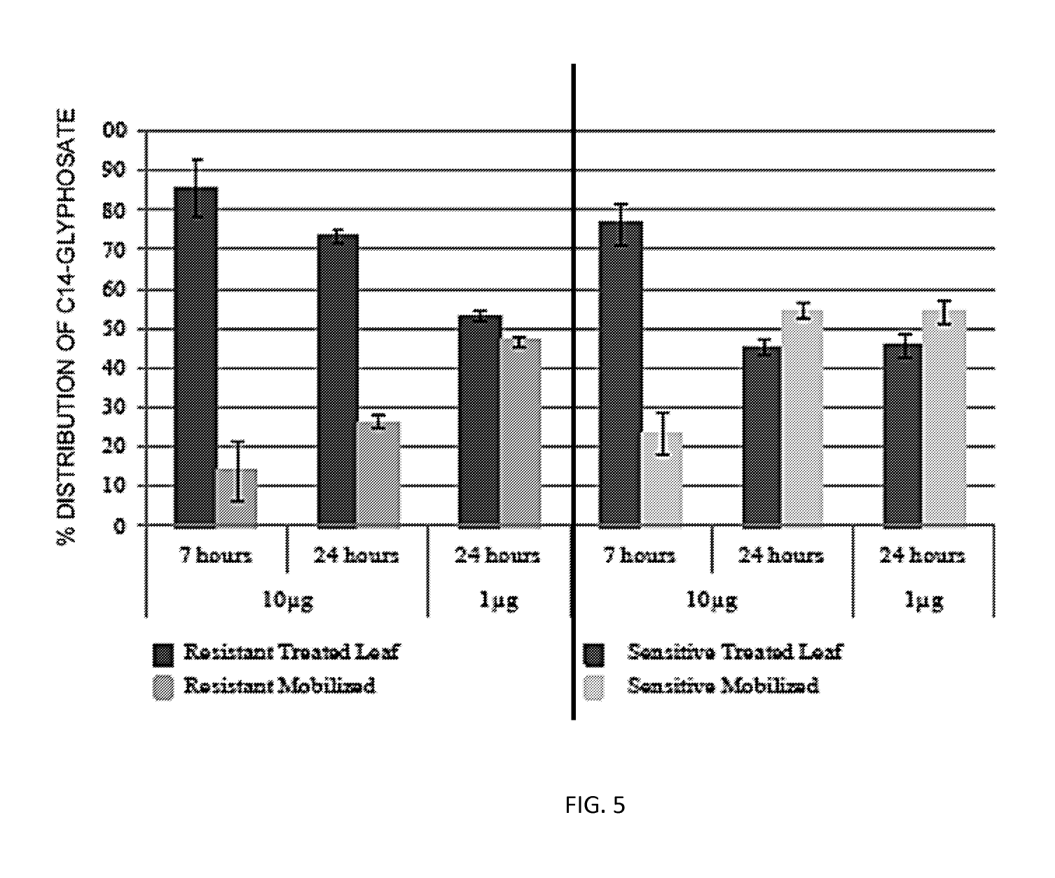

In a further study, ¹⁴C-glyphosate uptake and translocation showed glyphosate resistant plants translocated 28% of absorbed glyphosate whereas glyphosate-sensitive plants translocated 54% of absorbed glyphosate, as shown in FIG. 5. In the study, the treated leaf included two mature leaves treated with ¹⁴C-glyphosate. Mobilized distribution includes untreated mature leaves, meristems and roots. This indicates that the rapid necrosis of source leaves results in reduced glyphosate movement to sink tissues and contributes to the survival of glyphosate-resistant plants.

Nuclear Magnetic Resonance Imaging in Resistant and Sensitive A. trifida:

Nuclear magnetic resonance (NMR) imaging studies using glyphosate infiltrated leaves indicated rapid cell death occurred in glyphosate-resistant plants within 16 hours of glyphosate exposure compared to limited cell death in glyphosate-sensitive plants. An in vitro ³¹P NMR study of glyphosate sensitive and an in vitro ³¹P NMR study of glyphosate-resistant plants were conducted. The loss of the pH gradient, dumping of glyphosate, dumping of PO₄, and declining ATP levels showed that the glyphosate beyond 16 hours is resulting in cell death in the glyphosate-resistant plant tissue while the glyphosate-sensitive tissue remains viable.

Mass Spectroscopy of Resistant and Sensitive A. trifida:

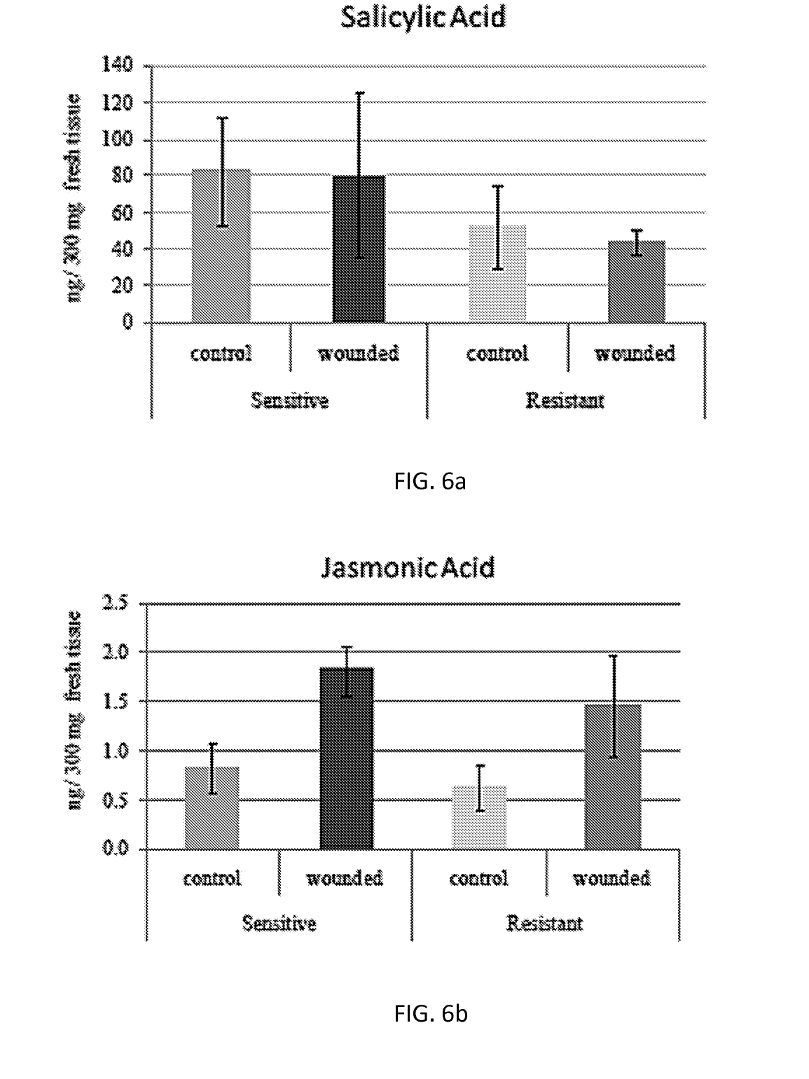

To further investigate if the rapid necrosis mechanism was associated with a hypersensitive disease response pathway in plants, mass spectroscopy was used. No difference between glyphosate-resistant and glyphosate-sensitive plants in terms of salicylic acid (SA) (FIG. 6a) and jasmonic acid (JA) (FIG. 6b) levels after mechanical wounding was found, as shown in FIGS. 6a and 6b.

In an embodiment, the plants are grown as described for dose response experiments. Tissue samples are harvested pre- and post-glyphosate treatment and frozen in liquid nitrogen. For analysis, 2 ml MeOH/0.1 g and a dihydro-JA internal standard are added to ground tissue and stored overnight at 4° C. shaking periodically. After 24 hours, 1.2 ml ddH2O/0.1 g and 1 ml chloroform/0.1 g is added to samples and samples placed back in 4° C. for 24 hours to obtain phase separation. The polar, top layer of the solution is dried and resuspended in 100 μl mobile phase diluents (80% ddH2O/20% acetonitrile). Samples are sonicated, centrifuged at 4° C. at 12,000 rpm and supernatant transferred to HPLC vial. Measurements of SA and JA are determined by LC-MS/MS on an Agilent 1200 system (Palo Alto, Calif.) using a Waters Xterra MS C18 column (5 μm, 150×2.1 mm i.d). Data are analyzed using Agilent MassHunter software (version B.02). SA and JA quantification is accomplished using a nine level calibration curve (range 0.5-200 ng/mL) constructed with authentic standards.

Plant Fitness of Resistant and Sensitive A. trifida:

A weed's ability to establish, survive and reproduce successfully is termed weed fitness (Maxwell et al., 1990). Jasieniuk et al. (1996) described plant fitness as “the potential evolutionary success of a phenotype based on its survival and reproductive success with a fit plant contributing the majority of genetic material to the next generation”. To successfully access fitness of one particular species one should include germination, long-term seed viability, survival, establishment, fecundity, plant growth, competitive ability during vegetative growth stages and seed output (Radosevich et al., 1997). Fitness is one of the important factors influencing the appearance and persistence of herbicide-resistant biotypes in the absence of herbicide (Maxwell et al., 1990). To assess fitness, the replacement series (RS) method designed by de Wit (1960) has been extensively used in competition studies between two species or two biotypes of the same species. The replacement series for two species or biotypes consists of a pure stand or monoculture of each of the species and a range of mixtures in which species are sown at a proportion P and P−1 of their pure stand densities respectively. The performance of a mixture, compare with that of pure stand, has been widely used to assess relative competitiveness and aggressiveness of each species.

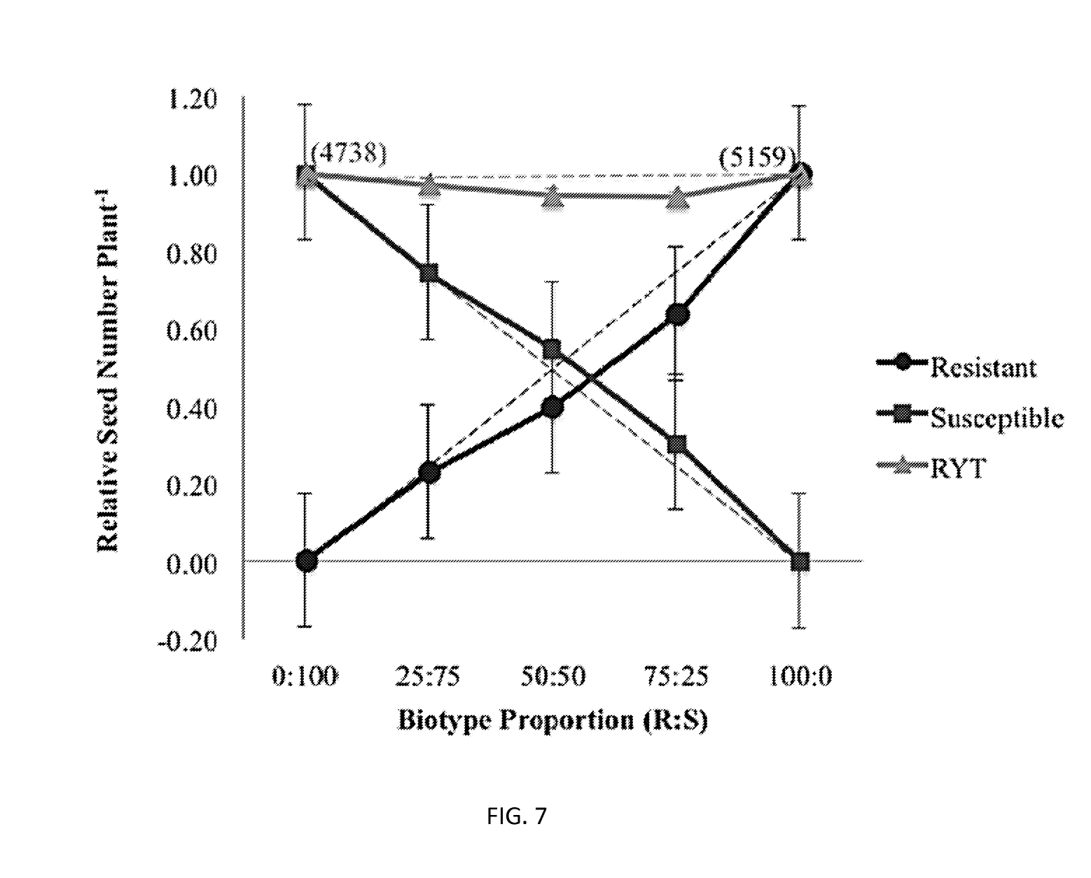

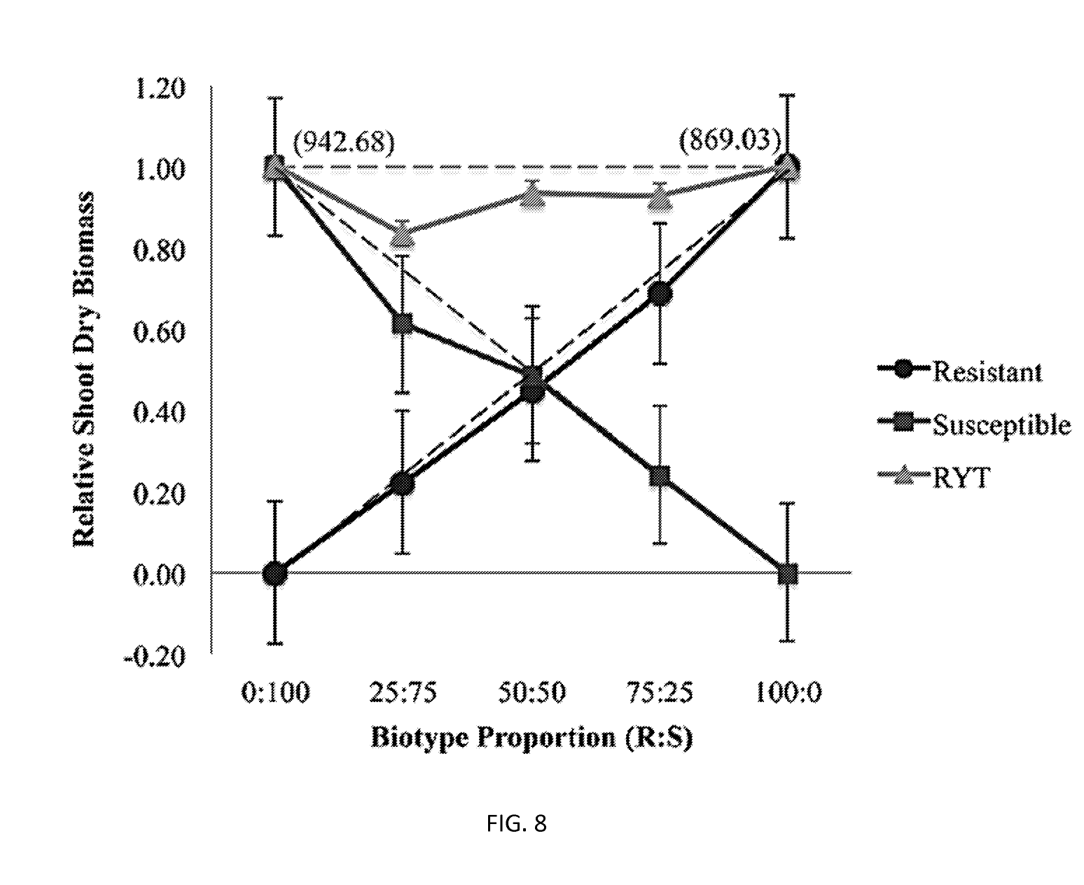

As shown in FIGS. 7 and 8, a study using growth analysis of the fitness of both resistant and sensitive plants grown in monoculture showed that both types had the same growth rate, biomass, and reproductive capacity and showed no difference in competitive ability when grown in replacement series experiments. FIG. 7 provides the relative seed numbers produced by glyphosate-resistant and sensitive biotypes grown at total input density of 4 plants 0.25 m². FIG. 8 provides the relative shoot dry biomass of resistant and glyphosate susceptible giant ragweed biotypes grown at total input density of 4 plants 0.25 m².

H₂O₂ Accumulation on Resistant and Sensitive A. trifida:

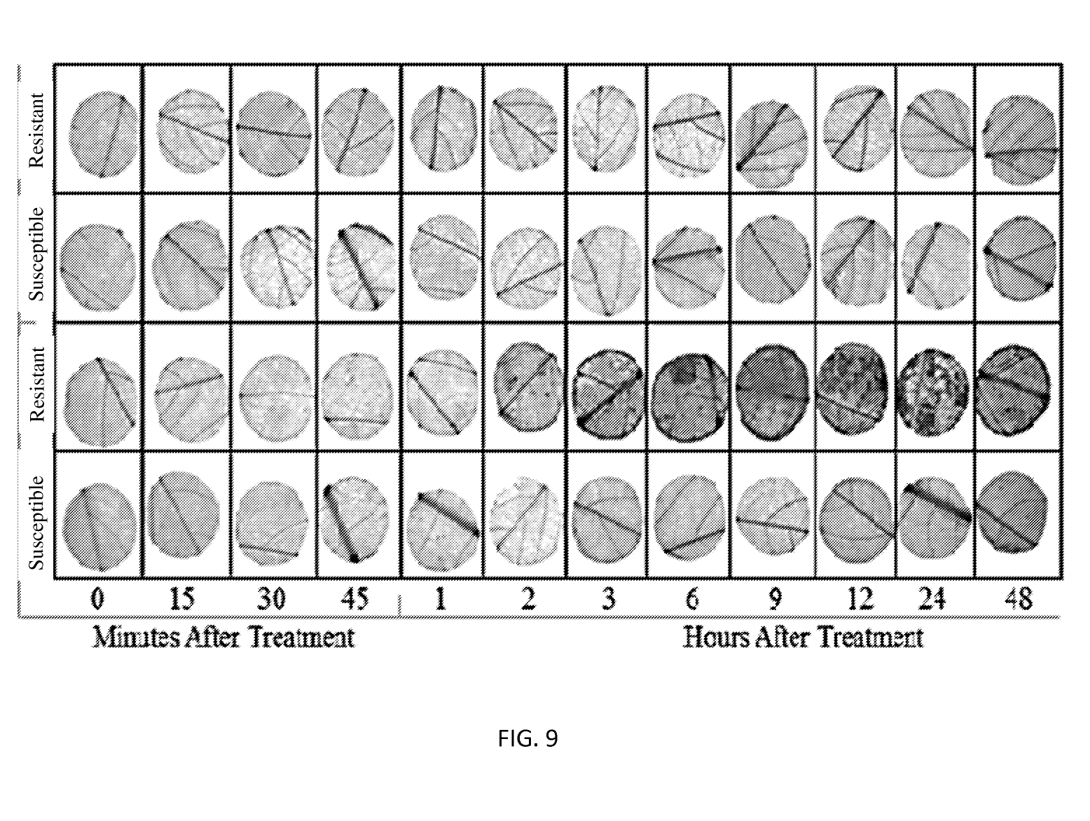

FIG. 9 provides the results of a study comparing H₂O₂ accumulation on glyphosate treated mature and immature glyphosate-resistant and glyphosate-sensitive leaves using DAB staining. The treatment resulted in significant H₂O₂ accumulation in mature leaves of glyphosate-resistant plants within 2 hrs of glyphosate treatment. In contrast, glyphosate sensitive leaves showed no H₂O₂ production. These results show that glyphosate-resistant plants exhibit rapid cell death in response to glyphosate treatment. This response likely limits glyphosate translocation to sink tissues with high meristematic activity and therefore allows plant survival as the meristems are able to regrow new leaves after glyphosate treatment. In FIG. 9, a comparison of H₂O₂ accumulation on glyphosate treated immature (upper two rows) and matures GS- and GR-GRWleaves (lower two rows) using DAB staining is provided. Visual detection of H₂O₂ in leaves after DAB staining showed that mature leaves of glyphosate-resistant plants produce H₂O₂ at 2 hrs after glyphosate treatment, and immature and leaves from glyphosate sensitive plants did not.

In an embodiment, the H₂O₂ presence is visually detected after 3,3′-diaminobenzidine (DAB) staining using procedures described by Thordal-Christensen et al., (1977). Glyphosate-resistant and glyphosate-sensitive biotype plants are propagated and maintained as described and treated with glyphosate solutions at 0× and 2× field rate (1× field rate=0.7 kg ae ha-1) rates as described. At 15, 30, 45 minutes and 1, 2, 3, 6, 9, 12, 24, and 48 hours after glyphosate treatment, two leaf discs from the lowest fully expanded leaves (first node) and two leaf discs (16 mm diameter) from young leaves on the 4-5th node are collected and combined among 4 replicate plants within biotype category and treated as one sample. At each harvested time, leaf discs are floated in a solution of DAB-Hydrochloric acid (HCl) (1 mg/ml) (Sigma, St. Louis, Mo.) in a beaker at room temperature and allowed to absorb the solution through the cut ends. After 3 hours of incubation, leaf discs are decolorized by boiling in 90% ethanol for ten minutes to remove chlorophyll, before visual examination. Brown precipitates form at sites of H₂O₂ accumulation and data collected will be by visual detection of H₂O₂ accumulation as noted by intensity of brown precipitates on treated leave discs. The hypersensitive reaction (HR) has been used as a visual marker of biotic interactions and induction of pathogen response (PR) genes (S) but this has been also shown in plant responses to abiotic stresses such as excess excitation energy (EEE) (Muhlenbock et al., 2008).

Expression Levels in Resistant and Sensitive A. trifida:

In a further study, leaf material was harvested from non-treated plants, as well as 3 hrs, 6 hrs and 12 hrs after glyphosate treatment in resistant and sensitive A. trifida. A modified protocol after Eggermont et al., (1996) was used to extract total RNA from mature leaves of resistant and sensitive plants grown in a growth room under controlled environmental conditions (16:8 hrs, light:dark). Leaf disks of 2 cm diameter from the first fully developed leaf were punched out, frozen in liquid nitrogen, and total RNA extracted in 2 ml test tube scale. An extraction buffer combination of SDS and phenol:chloroform mix was used for primary extraction. RNA was purified with subsequent chloroform extractions and LiCl precipitations. DNA contaminations were removed via DNaseI (RNase-free, NEB Biolabs, Ipswich, Mass.) treatment.

RNA concentrations were determined with a Nanodrop photometer and the quality assessed with the RNA 6000 nanochip of the Agilent Bioanalyzer. Samples with RIN numbers (RNA Integrity Number) bigger than 8 were used for library construction. Sequencing libraries were constructed using the Illumina TruSeq RNA library kit with paired-end barcoding. Steps in this procedure include isolation of poly-A containing mRNA and fragmentation to small pieces which were transcribed into first and second strand cDNA and ligated to adapter oligonucleotides and subsequently amplified with PCR.

RNA was assembled from paired-end reads using the Trinity package (Grabherr et al., 2011). Between 31×106 and 88×106 raw reads of 98 bp average lengths were generated via Illumina sequencing from the different RNA samples. The resulting assembly contained 249598 sequences derived from 124264 assemblies. Reads were mapped to the assembly sequences and the number of read counts per million transcripts (CPM) determined using the RSEM package (Li and Dewey, 2011).

The Fragments Per Kilobase of exon per Million fragments mapped (FPKM) values were normalized using control genes. A list of rice genes with stable expression levels over many conditions were identified by Jain (2009). A collection of 25 genes with stable expression patterns given in the paper were used as queries in a tblastn search against the assembled ragweed sequences. Twenty-one of the initial 25 had matches in the A. trifida sequence. The expression levels (CPM, log counts per million) for these genes in both the resistant and sensitive varieties were compared across all four time points; only twelve genes showed a relatively stable CPM, while the rest of the genes varied across the time points (more than 1.5 fold up or down). These twelve genes are the standard genes.

To normalize the expression levels, a scale factor was determined for each standard gene with respect to the time zero point (scale=CPMt/CPM0). An average scale factor for each time point was then calculated as the simple average of the scale factors for each of the standard genes at each time point. By definition, the scale factor for the zero time point is one, corrected CPM for all genes were then calculated by multiplying the raw expression level by the scale factor for the respective time point.

The counts for each trinity assembly were summed for all sub-components and isoforms to produce gene level counts. Assemblies with less than 1 CPM counts in all time points were considered unobserved and excluded from further analysis. This left a total of 35467 assemblies. Expression ratios were then calculated for each assembly, comparing the expression levels in the glyphosate-resistant and glyphosate-sensitive strains at each time point (e.g., R3/S3). Numbers larger than 1 therefore reflect genes (assemblies) with higher expression in the resistant variety. Because there are no replicates in this experiment, the expression values are expected to be quite variable. All values were adjusted by the addition of a constant prior of 0.5 CPM before calculating expression ratios. Assemblies with log-expression ratios greater than 4, or less than −4 were further examined.

As can be seem from Tables 1-4, more than 35,000 gene hits in the Arabidopsis genome were annotated from the A. trifida sequences for all time points. The normalized expression levels of plants before treatment were compared against each other. In the cases of samples from plants post treatment the comparisons of expression levels were done between pre- and post-treatment plants. The expression of the majority of all genes is unchanged between pre- and post-treatment (25,000 to 33,000 genes) plants. Genes that are up-regulated in one of the two biotypes, but unchanged in the other biotype are also significant (550 to 5471 genes). Genes that show an opposite expression pattern (up-regulated in one biotype and down-regulated in the other biotype make up only a small fraction of the transcriptomes (18 to 329). When the transcriptomes of plants of both biotypes pre-treatment are compared, a significant number of genes can be found that are down-regulated in glyphosate-resistant plants in comparison to glyphosate-sensitive plants.

In Tables 1-4, expression analysis of up- and down-regulated genes in glyphosate-resistant and glyphosate-sensitive biotypes of A. trifida pre- and post-treatment was performed. Plants were treated with 1× field rate and samples collected for RNA sequencing experiments after 3 hrs, 8 hrs and 12 hrs. (+) signifies up-regulated genes, (−) signifies down-regulated genes and (=) signifies genes with no significant change in expression pattern.

TABLE 1
Pre-treatment comparison of expression levels in glyphosate-resistant and
glyphosate-sensitive A. trifida

| | |
|---|---|
| Overexpression in glyphosate-resistant A. trifidat | 318 |
| Equivalent expression in glyphosate-resistant and | 35079 |
| glyphosate-sensitive A. trifida | |
| Overexpression in glyphosate-sensitive A. trifida | 70 |

TABLE 2

| Pre-treatment comparison of | | | | |
|---|---|---|---|---|
| expression levels in glyphosate-resistant | | Glyphosate-sensitive A. trifida | | |
| and glyphosate-sensitive A. trifida | | Overexpression | Equivalent | Underexpression |
| Glyphosate-resistant | Overexpression | 62 | 550 | 31 |
| A. trifida | Equivalent | 552 | 33020 | 1014 |
| | Underexpression | 18 | 181 | 39 |

TABLE 3

| | | Glyphosate-sensitive A. trifida | | |
|---|---|---|---|---|
| Eight hours post-treatment | | Overexpression | Equivalent | Underexpression |
| Glyphosate-resistant | Overexpression | 101 | 3342 | 323 |
| A. trifida | Equivalent | 2643 | 26654 | 597 |
| | Underexpression | 58 | 1632 | 117 |

TABLE 4

| | | Glyphosate-sensitive A. trifida | | |
|---|---|---|---|---|
| Twelve hours post-treatment | | Overexpression | Equivalent | Underexpression |
| Glyphosate-resistant | Overexpression | 412 | 5471 | 329 |
| A. trifida | Equivalent | 1273 | 25339 | 696 |
| | Underexpression | 22 | 1710 | 215 |

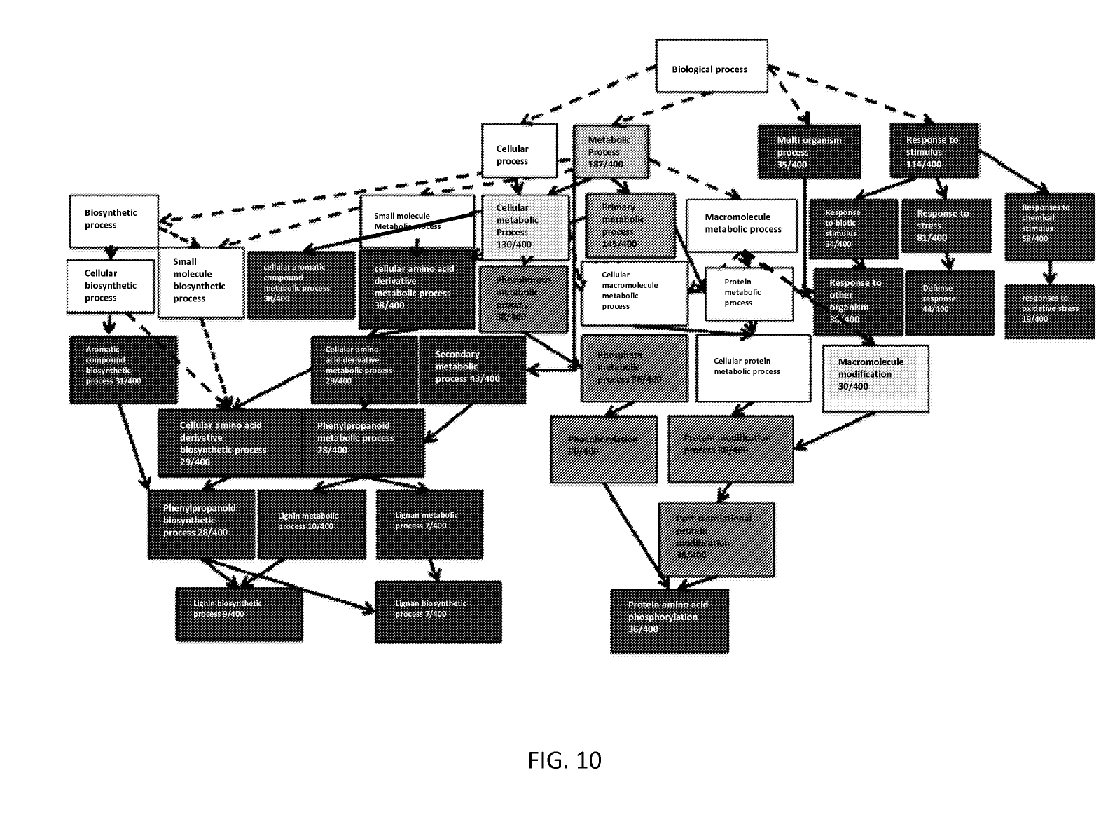

A further analysis concerning putative functions of these de-regulated genes unexpectedly indicated that a great number of these down-regulated genes were identified as involved in processes such as defense response, response to oxidative stress, response to biotic stimulus and response to chemical stimulus, as shown in FIG. 10. GO (gene ontology) analysis of down-regulated expression of genes in glyphosate-resistant plants under non-selective conditions is provided. The enrichment of GO-terms in the biological process category is shown in FIG. 10. Statistical significance is signified via color code (very high=red, high=orange, medium=yellow, low=white). GO description and mapping genes in the query list are indicated in boxes. Only GO terms with a p-value<1e⁻⁷ are included.

The analysis was performed using the GO-term (gene ontology) database agriGO (Zhou et al., 2010, http://bioinfo.cau.edu.cn/agriGO/analysis.php). The enrichment of the found Go terms for these down-regulated genes in glyphosate-resistant plants is highly significant (pvalue<1e⁻⁷). Without wishing to be bound by theory, the results suggest that glyphosate resistant plants are not only reacting to treatments with glyphosate in a manner resembling pathogen defense reactions but are already primed with a down-regulation of a significant number of genes involved in this process. In other words, the glyphosate-resistant plants are predetermined to react more significant to particular stresses than glyphosate-sensitive plants. This assumption was further strongly supported by the analysis of the functional enrichment of upregulated gene expression in glyphosate-resistant plants post-treatment. Again a dramatic upregulation of the expression of genes significant for responses to biotic stress and pathogen attack was found.

Method of Identifying Herbicide-Resistant Plants:

In some embodiments, the results disclosed herein are used to develop quantitative reverse-transcription PCR (qRT-PCR) based expression markers that allow rapid characterization of glyphosate-resistant plants under field conditions. Results of the expression level pilot study show that a number of genes are dramatically down-regulated in non-treated glyphosate-resistant plants in comparison to non-treated glyphosate-sensitive plants. Expression levels of differentially expressed genes differ up to 1000 fold from glyphosate-resistant to glyphosate-sensitive plants. Genes of this class provide the opportunity to develop a set of PCR primers that allow a rapid detection of glyphosate-resistant plants grown under field conditions.

In one embodiment, genes have been identified that are over-expressed in glyphosate-resistant plants compared to glyphosate-sensitive plants prior to treatment with an herbicide. Table 5 provides the results of gene sequences that exhibit over-expression in glyphosate-resistant plants compared to glyphosate-sensitive plants. As can be seen, forty-seven sequences are disclosed that exhibit at least 2-fold expression in resistant plants compared to sensitive plants.

TABLE 5
Genes over-expressed in resistant plants compared to sensitive plants

| | | Relative | Relative | Ratio of |
|---|---|---|---|---|
| | | Expression Level | Expression | expression level |
| | | in Resistant | Level in | for resistant plants |
| SEQ ID NO: | Sequence Name | Plants | Sensitive Plants | to sensitive plants |
| 1 | comp148939_c0 | 11598.425 | 432.035 | 26.84603099 |
| 2 | comp166081_c0 | 5941.03 | 225.52 | 26.34369457 |
| 3 | comp149865_c0 | 940.955 | 122.76 | 7.664996742 |
| 4 | comp142951_c0 | 1364.855 | 208.935 | 6.532438318 |
| 5 | comp159731_c0 | 1459.94 | 297.58 | 4.906042073 |
| 6 | comp167561_c0 | 776.35 | 162.505 | 4.777391465 |
| 7 | comp158185_c0 | 869.37 | 189.77 | 4.581177215 |
| 8 | comp171245_c0 | 1162.93 | 285.295 | 4.076236878 |
| 9 | comp168138_c0 | 406.48 | 106 | 3.834716981 |
| 10 | comp171200_c0 | 432.035 | 127.195 | 3.396635088 |
| 11 | comp143198_c0 | 484.48 | 147.015 | 3.295446043 |
| 12 | comp163779_c0 | 746.745 | 228.615 | 3.26638672 |
| 13 | comp155437_c0 | 354.1 | 108.78 | 3.255193969 |
| 14 | comp168558_c0 | 325.385 | 100.985 | 3.222112195 |
| 15 | comp166444_c0 | 350.135 | 111.66 | 3.135724521 |
| 16 | comp131689_c0 | 358.86 | 115.015 | 3.120114768 |
| 17 | comp170147_c0 | 411.47 | 132.99 | 3.093992029 |
| 18 | comp149139_c0 | 579.86 | 196.51 | 2.950791308 |
| 19 | comp171170_c0 | 1445.24 | 502.305 | 2.877216034 |
| 20 | comp164900_c0 | 353.295 | 131.27 | 2.691361316 |
| 21 | comp150858_c0 | 301.47 | 112.61 | 2.677115709 |
| 22 | comp166920_c0 | 319.045 | 121.245 | 2.631407481 |
| 23 | comp165438_c0 | 518.53 | 201.41 | 2.574499777 |
| 24 | comp168822_c0 | 1585.75 | 617.485 | 2.568078577 |
| 25 | comp154444_c0 | 508.855 | 201.085 | 2.530546784 |
| 26 | comp157635_c0 | 544.375 | 215.215 | 2.529447297 |
| 27 | comp163792_c0 | 956.795 | 380.07 | 2.517417844 |
| 28 | comp172768_c0 | 615.25 | 249.985 | 2.461147669 |
| 29 | comp95569_c0 | 312.45 | 130.375 | 2.396548418 |
| 30 | comp158296_c0 | 1860.76 | 789.81 | 2.355959028 |
| 31 | comp171533_c0 | 415.035 | 179.17 | 2.316431322 |
| 32 | comp171767_c0 | 301.75 | 130.275 | 2.316254078 |
| 33 | comp163200_c0 | 1238.325 | 537.225 | 2.305039788 |
| 34 | comp144782_c0 | 501.785 | 218.145 | 2.300236082 |
| 35 | comp164521_c0 | 230.98 | 102.205 | 2.259967712 |
| 36 | comp160021_c0 | 219.495 | 100.9 | 2.175371655 |
| 37 | comp157764_c0 | 239.915 | 111.075 | 2.15993698 |
| 38 | comp158513_c0 | 228.71 | 106.075 | 2.156115956 |
| 39 | comp156192 c0 | 239.2 | 111.715 | 2.14116278 |
| 40 | comp154139_c0 | 401.35 | 189.345 | 2.119675724 |
| 41 | comp157072_c0 | 924.69 | 438 1 5 | 2.110562054 |
| 42 | comp155926_c0 | 268.18 | 128.335 | 2.089687147 |
| 43 | comp170115_c0 | 262.07 | 126.385 | 2.073584682 |
| 44 | comp167867_c0 | 308.525 | 148.79 | 2.073560051 |
| 45 | comp151863_c0 | 366.255 | 178.19 | 2.055418374 |
| 46 | comp151483_c0 | 273.635 | 134.85 | 2.029180571 |
| 47 | comp158852 _c0 | 278.695 | 138.195 | 2.01667933 |

Similarly, in another embodiment, genes have been identified that are underexpressed in glyphosate-resistant plants compared to glyphosate-sensitive plants prior to treatment with an herbicide. Table 6 provides the results of gene sequences that exhibit underexpression in glyphosate-resistant plants compared to glyphosate-sensitive plants. As shown, seventy-three gene sequences are disclosed that exhibit at least 2-fold expression in sensitive plants compared to resistant plants.

TABLE 6
Genes over-expressed in sensitive plants compared to resistant plants

| | | Relative | Relative | Ratio of |
|---|---|---|---|---|
| | | Expression Level | Expression Level | expression level |
| | | in Resistant | in Sensitive | for sensitive plants |
| SEQ ID NO | Sequence Name | Plants | Plants | to resistant plants |
| 48 | comp161591_c0 | 130.02 | 1680.68 | 12.92631903 |
| 49 | comp165624_c0 | 320.485 | 3110.785 | 9.706491723 |
| 50 | comp144176_c0 | 154.065 | 1185.42 | 7.69428488 |
| 51 | comp138113_c0 | 589.545 | 3256.395 | 5.523573264 |
| 52 | comp150391_c0 | 202.825 | 1008.48 | 4.972168125 |
| 53 | comp165059_c0 | 323.545 | 1521.35 | 4.702127988 |
| 54 | comp163658_c0 | 104.865 | 446.485 | 4.157712297 |
| 55 | comp1542.2_c0 | 181.615 | 696.86 | 3.837017867 |
| 56 | comp163561_c0 | 201.085 | 733.655 | 3.648481985 |
| 57 | comp159744_c0 | 104.625 | 354.1 | 3.384468339 |
| 58 | comp153870_c0 | 330.69 | 1091.5 | 3.300674348 |
| 59 | comp168083_c0 | 112.25 | 366.255 | 3.26285078 |
| 60 | comp160035_c0 | 161.635 | 506.38 | 3.13286076 |
| 61 | comp119862_c0 | 99.765 | 306.295 | 3.070164887 |
| 62 | comp170964_c0 | 106.32 | 326.115 | 3.0672.9684 |
| 63 | comp106640_c0 | 108.08 | 323.08 | 2.989267209 |
| 64 | comp157816_c0 | 174.05 | 512.495 | 2.94452735 |
| 65 | comp160167_c0 | 122 | 358.86 | 2.9414741 |
| 66 | comp147178_c0 | 226.14 | 647.19 | 2.86189908 |
| 67 | comp159753_c0 | 445.905 | 1270.165 | 2.848510333 |
| 68 | comp157941_c0 | 106.895 | 301.75 | 2.822863558 |
| 69 | comp158289_c0 | 554.15 | 1544.335 | 2.78685374 |
| 70 | comp148517_c0 | 386.535 | 1062.78 | 2.749505219 |
| 71 | comp165052_c0 | 216.145 | 589.545 | 2.72754401 |
| 72 | comp163411_c0 | 127.195 | 344.765 | 2.710523212 |
| 73 | comp163685_c0 | 766.17 | 2037.275 | 2.659037811 |
| 74 | comp142420_c0 | 120.12 | 312.575 | 2.602189477 |
| 75 | comp167693_c0 | 292.31 | 757.755 | 2.592299271 |
| 76 | comp150685_c0 | 101.115 | 262.07 | 2.591801414 |
| 77 | comp161582_c0 | 118.35 | 303.34 | 2.56305623 |
| 78 | comp163279_c0 | 3040.815 | 7683.89 | 2.526917948 |
| 79 | comp164418_c0 | 789.39 | 1975.19 | 2.502172564 |
| 80 | comp134770_c0 | 128.675 | 318.365 | 2.474179133 |
| 81 | comp153258_c0 | 141.885 | 350.86 | 2.47284729 |
| 82 | comp151938_c0 | 502.305 | 1229.18 | 2.44708966 |
| 83 | comp151909_c0 | 145.72 | 355.31 | 2.438306341 |
| 84 | comp158705_c0 | 100.985 | 244.86 | 2.424716542 |
| 85 | comp153385_c0 | 214.53 | 518.53 | 2.417051228 |
| 86 | comp104360 c0 | 4842.37 | 11598.425 | 2.395195947 |
| 87 | comp157097_c0 | 120.825 | 289.055 | 2.392343 |
| 88 | comp164409_c0 | 101.375 | 242.34 | 2.39053021 |
| 89 | comp167277_c0 | 101.75 | 239.915 | 2.357886978 |
| 90 | comp139757_c0 | 175.5 | 413.65 | 2.356980057 |
| 91 | comp133103_c0 | 202.235 | 474.945 | 2.348480728 |
| 92 | comp163835_c0 | 271.15 | 634.64 | 2.340549511 |
| 93 | comp169260_c0 | 181.14 | 418.35 | 2.309539583 |
| 94 | comp159995_c0 | 100.37 | 230.705 | 2.298545382 |
| 95 | comp142668_c0 | 198.295 | 455.67 | 2.297939938 |
| 96 | comp161537_c0 | 247.895 | 566.355 | 2.28465681 |
| 97 | comp146816_c0 | 120.25 | 272.415 | 2.265405405 |
| 98 | comp156794_c0 | 107.535 | 236.885 | 2.202864184 |
| 99 | comp160421_c0 | 1762.54 | 3873.23 | 2.197527432 |
| 100 | comp157651_c0 | 188.015 | 413.03 | 2.196792809 |
| 101 | comp162277_c0 | 305.095 | 668.115 | 2.189858896 |
| 102 | comp162337_c0 | 362.31 | 792.89 | 2.188429798 |
| 103 | comp154823_c0 | 104.285 | 226.915 | 2.175912164 |
| 104 | comp167344_c0 | 206.17 | 445.905 | 2.162802542 |
| 105 | comp157026_c0 | 234.255 | 505.85 | 2.159398946 |
| 106 | comp170831_c0 | 291.145 | 623.595 | 2.148740318 |
| 107 | comp167394_c0 | 169.12 | 362.31 | 2.142324976 |
| 108 | comp155911_c0 | 652.165 | 1393.455 | 2.136660201 |
| 109 | comp156896_c0 | 661.995 | 1409.78 | 2.129593124 |
| 110 | comp171464_c0 | 279.295 | 593.47 | 2.124885873 |
| 111 | comp164879_c0 | 110.07 | 233.28 | 2.119378577 |
| 112 | comp169469_c0 | 207.545 | 437.265 | 2.106844299 |
| 113 | comp166108_c0 | 347.69 | 731.06 | 2.10262015 |
| 114 | comp167365_c0 | 130.375 | 273.635 | 2.098830297 |
| 115 | comp170210_c0 | 113.195 | 234.875 | 2.074959141 |
| 116 | comp164800_c0 | 145.56 | 297.135 | 2.041323166 |
| 117 | comp160915_c0 | 393.1 | 798.56 | 2.031442381 |
| 118 | comp151923_c0 | 460.865 | 934.16 | 2.026971022 |
| 119 | comp151839_c0 | 148.73 | 298.885 | 2.00958112 |
| 120 | comp167225_c0 | 212.525 | 426 | 2.004470062 |

In an embodiment, one skilled in the art could develop primers to identify the expression level of one or more of these genes. In some embodiments, the expression level is quantified using a technique such as qRT PCR. Table 7 discloses primers developed for identifying genes in Table 5 that have greater than 4-fold overexpression in resistant plants compared to sensitive plants. Table 8 discloses primers developed for identifying genes in Table 6 that have greater than 4-fold overexpression in sensitive plants compared to resistant plants. In some embodiments, the primers are used in a PCR analysis with template DNA from one or more plants in the sample. The primer name in Tables 7 and 8 indicates the corresponding gene in Tables 5 and 6.

TABLE 7
Primers developed for genes overexpressing in resistant plants
compared to sensitive plants.

| SEQ ID NO: | Primer name | Sequence |
|---|---|---|
| 121 | comp148939_c0_seq1_FOR1 | ACCGAACAACCACAAGGTCAA |
| 122 | comp148939_c0_seq1_REV1 | GGGCTGCATAATGAGCATCA |
| 123 | comp148939_c0_seq1_FOR2 | CCGAACAACCACAAGGTCAA |
| 124 | comp148939_c0_seq1_REV2 | GCGATTGGGCTGCATAATG |
| 125 | comp148939_c0_seq1_FOR3 | CGAACAACCACAAGGTCAACA |
| 126 | comp148939_c0_seq1_REV3 | CATCCACTCTCCGACACAACA |
| 127 | comp166081_c0_seq1_FOR1 | CCCTAGCTCCAACGCATCAA |
| 128 | comp166081_c0_seq1_REV1 | GGAGTCTCTTAACGGCGGATAA |
| 129 | comp166081_c0_seq1_FOR2 | ACCCCCACCCCTTCTGATC |
| 130 | comp166081_c0_seq1_REV2 | CCGACGAGGTTTTGACTGGAT |
| 131 | comp166081_c0_seq1_FOR3 | CCATACCCCCACCCCTTCT |
| 131 | comp166081_c0_seq1_REV3 | CGTTTTCCGACGAGGTTTTG |
| 133 | comp149865_c0_seq1_FOR1 | TTCGCGGGTGTACCAACAA |
| 134 | comp149865_c0_seq1_REV1 | CAGAAAGCCACTTGCCCTAAA |
| 135 | comp149865_c0_seq1_FOR2 | CGCGGGTGTACCAACAACTA |
| 136 | comp149865_c0_seq1_REV2 | CAAAGGCCCCTAAGCCACAT |
| 137 | comp149865_c0_seq1_FOR3 | GCGGGTGTACCAACAACTAGGT |
| 138 | comp149865_c0_seq1_REV3 | GGCCCCTAAGCCACATAAACA |
| 139 | comp142951_c0_seq1_FOR1 | GCAACACCACCATGCATGAG |
| 140 | comp142951_c0_seq1_REV1 | AACTCCCATCAATTCCCACTTG |
| 141 | comp142951_c0_seq1_FOR2 | CGGTGCTACAGAACCCACAA |
| 142 | comp142951_c0_seq1_REV2 | TTGCTGGTGGCGTTCCTAAC |
| 143 | comp142951_c0_seq1_FOR3 | ACGGTGCTACAGAACCCACAA |
| 144 | comp142951_c0_seq1_REV3 | ATCAAGGCCTCATGCATGGT |
| 145 | comp159731_c0_seq1_FOR1 | TGAGGAAAATGCTTCCATCGT |
| 146 | comp159731_c0_seq1_REV1 | GACAGACATCACCCGCCTAAC |
| 147 | comp159731_c0_seq1_FOR2 | AAGTTAGGCGGGTGATGTCTGT |
| 148 | comp159731_c0_seq1_REV2 | CGCTGAAGATGCACGGAGAA |
| 149 | comp159731_c0_seq1_FOR3 | GGGACTTCAGCTGTGATTTGG |
| 150 | comp159731_c0_seq1_REV3 | CAGACATCACCCGCCTAACTTT |
| 151 | comp167561_c0_seq1_FOR1 | CCTTGACTTCTTGGCGTAGTACA |
| 152 | comp167561_c0_seq1_REV1 | GCGCGGGTTAAAAAAAACCT |
| 153 | comp167561_c0_seq1_FOR2 | CCCTTGACTTTCTTGGCGTAGT |
| 154 | comp167561_c0_seq1_REV2 | GGTGGCGCGGGTTAAAA |
| 155 | comp167561_c0_seq1_FOR3 | CTCGAACGCTAAGCTCATTTCA |
| 156 | comp167561_c0_seq1_REV3 | ATCACAGCATATCCACGCCTAGA |
| 157 | comp158185_c0_seq1_FOR1 | TGAAGCTGCTTAGCGAATTCC |
| 158 | comp158185_c0_seq1_REV1 | ACATCTCTGACATCCCCGATCT |
| 159 | comp158185_c0_seq1_FOR2 | CTGGGCCTCATCAGCTAAGC |
| 160 | comp158185_c0_seq1_REV2 | GAGCATTACAAGAAGTGCATGGA |
| 161 | comp158185_c0_seq1_FOR3 | GAGTTGAAGCTGCTTAGCGAATT |
| 162 | comp158185_c0_seq1_REV3 | CATCTCCCCCAATCCAACAT |
| 163 | comp171245_c0_seq1_FOR1 | CCCTCAAAAGCAAAGCCATCT |
| 164 | comp171245_c0_seq1_REV1 | ACGTTCCTGAGCTTGCTGAGA |
| 165 | comp171245_c0_seq1_FOR2 | TGGTGATCAGCAGGAACATACG |
| 166 | comp171245_c0_seq1_REV2 | TGGGCTGCTTTGGAAAAACT |
| 167 | comp171245_c0_seq1_FOR3 | ACCAGCACGCTCCACAAGTC |
| 168 | comp171245_c0_seq1_REV3 | CGGCCAGATCCCATCACA |

TABLE 8
Primers developed for genes overexpressing in sensitive
plants compared to resistant plants

| SEQ ID NO: | Primer name | Sequence |
|---|---|---|
| 169 | comp161591_c0_seq1_FOR1 | CAAACCAGACAAGTTCACTTCAGAA |
| 170 | comp161591_c0_seq1_REV1 | CATAAGGCGGTGGAAGGATTC |
| 171 | comp161591_c0_seq1_FOR2 | GGTGAAACCACTTTTGGAAACC |
| 172 | comp161591_c0_seq1_REV2 | CAGCAGTTCTTTGTTTTCTTTTCAGT |
| 173 | comp161591_c0_seq1_FOR3 | TTCCAACCATTTAGTGCCACAA |
| 174 | comp161591_c0_seq1_REV3 | GTATGTATTTTGGTGTGCATATCATGTT |
| 175 | comp165624_c0_seq1_FOR1 | CATGTGCATTCAGGGACTCGTA |
| 176 | comp165624_c0_seq1_REV1 | TCACCGCTGATCCCAACAA |
| 177 | comp165624_c0_seq1_FOR2 | GGCCGACAGACGTACGTTCT |
| 178 | comp165624_c0_seq1_REV2 | CTTCTCAGGCTGAAACTGGTTGT |
| 179 | comp165624_c0_seq1_FOR3 | CACCAAACAGGCATGTGCAT |
| 180 | comp165624_c0_seq1_REV3 | TCTGCATCATCACCGCTGAT |
| 181 | comp144176_c0_seq1_FOR1 | CCCTCTGCCCCTACCGTACT |
| 182 | comp144176_c0_seq1_REV1 | TCAAATCCCAGGGCTCAACT |
| 183 | comp144176_c0_seq1_FOR2 | GCCACCTACAGACGCTTTACG |
| 184 | comp144176_c0_seq1_REV2 | AGCAGCCGCGGTAATACAGA |
| 185 | comp144176_c0_seq1_FOR3 | CTCCCCAGGCGGGATACTTA |
| 186 | comp144176_c0_seq1_REV3 | AGTCCTAGCCGTAAACGATGGA |
| 187 | comp138113_c0_seq1_FOR1 | CAGGGTTCCTGCTCTCAGCTT |
| 188 | comp138113_c0_seq1_REV1 | GATTTGTCCGGCTGTTGTGA |
| 189 | comp138113_c0_seq1_FOR2 | ATGTGCCACAGCATCAATGC |
| 190 | comp138113_c0_seq1_REV2 | GCATTGGATGAACCTTGAAGCT |
| 191 | comp138113_c0_seq1_FOR3 | TGGGACGAATGATGAATTTGG |
| 192 | comp138113_c0_seq1_REV3 | TTTGTCCGGCTGTTGTGATG |
| 193 | comp150391_c0_seq1_FOR1 | CGCGGGTGGATTCATGAG |
| 194 | comp150391_c0_seq1_REV1 | CCTGACGTAACGAGCACCATT |
| 195 | comp150391_c0_seq1_FOR2 | CCACGGCGTCAAAAAGCTTA |
| 196 | comp150391_c0_seq1_REV2 | TTCCTTTGCATTGTGAACATTGT |
| 197 | comp150391_c0_seq1_FOR3 | GATCCTCTGTGGTGGTTGCA |
| 198 | comp150391_c0_seq1_REV3 | GACGTAACGAGCACCATTTCAA |
| 199 | comp165059_c0_seq1_FOR1 | GTTCCAGAGCATCCAATGCAT |
| 200 | comp165059_c0_seq1_REV1 | GAAAATCTGTTTGCGGGACAA |
| 201 | comp165059_c0_seq1_FOR2 | GGTCTTCAGCACCACAAGCTT |
| 202 | comp165059_c0_seq1_REV2 | CTGCCGCTACTCACTTTGGTT |
| 203 | comp165059_c0_seq1_FOR3 | TCCAGAGCATCCAATGCATAAG |
| 204 | comp165059_c0_seq1_REV3 | TCGTCACAGAGACCGCGTACT |
| 205 | comp163658_c0_seq1_FOR1 | GCTTCAAGATTTGCCTGTGGTT |
| 206 | comp163658_c0_seq1_REV1 | AATCTCGTCATCAAACAAATCAAACT |
| 207 | comp163658_c0_seq1_FOR2 | TTGCAAAAAAGGACAGAAGTCAAG |
| 208 | comp163658_c0_seq1_REV2 | CCATGGTCTCATTGTTGATCTCA |
| 209 | comp163658_c0_seq1_FOR3 | CAACAATGAGACCATGGATGATG |
| 210 | comp163658_c0_seq1_REV3 | CTAACTTGTACCGCATTCAACGA |

The RNA to produce a template DNA for PCR may be prepared from a leaf punch as is known in the art. In some embodiments, a positive and/or negative control is included in the PCR run. In some embodiments, the PCR conditions are optimized for the one or more genes of interest and primers as is known in the art. In further embodiments, the results of the PCR analysis are quantitative and may be compared against a control or may be evaluated based on expected levels of absolute expression.

As is known in the art, the primers may be used to amplify and quantify expression of a gene in the plant tissue. In an embodiment, a selected primer pair is used to amplify and quantify the expression of a gene in a plant sample in order to determine the level of expression of the gene. For example, the gene associated with SEQ ID NO: 1, having the sequence name of comp148939_c0, is expressed at a ratio of 26.85:1 in resistant plants compared to sensitive plants before glyphosate is administered. By screening a plant sample with one or more of the primer pairs developed to amplify and quantify the gene associated with SEQ ID NO: 1, including SEQ ID NOS: 121 & 122, SEQ ID NOS: 123 & 124, or SEQ ID NOS: 125 and 126, one skilled in the art would be able to determine the expression level of the gene associated with SEQ ID NO: 1. In some embodiments, the expression level of the sample is compared to a control, such as a plant sample known to be sensitive to glyphosate. In other embodiments, the expression level of the sample is evaluated based on an absolute expression level. For example, the expression level may be compared to a predetermined threshold level. If the expression level is above the predetermined threshold level, then the plant sample is identified as being resistant to glyphosate. If the expression level is below the predetermined threshold level, then the plant sample is identified as being sensitive to glyphosate.

It should be understood that one or more of the genes associated with overexpression or under-expression may be evaluated for a single plant sample. For example, the gene associated with SEQ ID NO: 1 may be evaluated to determine if the plant sample overexpresses compares to a control and the gene associated with SEQ ID NO: 48 may be evaluated to determine if the plant sample under-expresses compared to a control.

In further embodiments, the expression level is quantified using a different technique. For example, specific primers can be used to amplify an integration fragment that can be used as a “specific probe” for identifying one or more of the over-expressed genes in a biological sample. Contacting nucleic acid of a biological sample with the probe under conditions that allow hybridization of the probe with its corresponding fragment in the nucleic acid results in the formation of a nucleic acid/probe hybrid. The formation of this hybrid can be detected (e.g. labeling of the nucleic acid or probe), whereby the formation of this hybrid indicates the presence of the over-expressed gene. Such identification methods based on hybridization with a specific probe (either on a solid phase carrier or in solution) have been described in the art. The specific probe is preferably a sequence that, under optimized conditions, hybridizes specifically to a region within the 5′ or 3′ flanking region of the gene of interest. Preferably, the specific probe comprises a sequence of between 50 and 500 bp, preferably of 100 to 350 bp which is at least 80%, preferably between 80 and 85%, more preferably between 85 and 90%, especially preferably between 90 and 95%, most preferably between 95% and 100% identical (or complementary) to the nucleotide sequence of a specific region. Preferably, the specific probe will comprise a sequence of about 15 to about 100 contiguous nucleotides identical (or complementary) to a specific region of the gene of interest. In some embodiments, techniques are used to quantify the expression of the gene of interest in order to determine overexpression or under-expression.

In some embodiments, antibody-based methods such as ELISA methods may be used to quantify the expression of the one or more genes of interest. The antibodies can be assayed for immunospecific binding by any method known in the art. The immunoassays which can be used include but are not limited to competitive and non-competitive assay systems using techniques such as western blots, radioimmunoassays, ELISA, sandwich immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Such assays are routine in the art (see, for example, Ausubel et al., eds, 1994, Current Protocols in Molecular Biology, Vol. 1, John Wiley & Sons, Inc., New York, which is incorporated by reference herein in its entirety).

Furthermore, detection methods specific for the one or more genes of interest which differ from PCR based amplification methods can also be developed using the specific sequence information provided herein. Such alternative detection methods include linear signalamplification detection methods based on invasive cleavage of particular nucleic acid structures, also known as Invader™ technology, (as described e.g. in U.S. Pat. No. 5,985,557 “Invasive Cleavage of Nucleic Acids”, U.S. Pat. No. 6,001,567 “Detection of Nucleic Acid sequences by Invader Directed Cleavage, incorporated herein by reference).

In further embodiments, protein products of the genes of interest are used to identify and/or quantify the presence of one or more genes of interest. For example, a test strip comprising an antibody may be developed that reacts to the one or more genes of interest in order to provide an estimate of the quantity of gene product produced and the likelihood that the plant is resistant to glyphosate.

In still further embodiments, the results are used to develop an integrated strategy to restore and improve glyphosate efficacy in glyphosate-resistant plants. For example, if the innate pathogen response is involved in glyphosate-resistance, providing a solution that comprises glyphosate and a compound to reduce the innate pathogen response may result in increased efficiency of glyphosate. In all experiments disclosed herein, glyphosate triggers a typical pathogen response reaction, which leads to abscission of mature leaves and subsequent detoxification of plant tissue exposed to the herbicide. Starting from this basic information a targeted screen for non-herbicidal supplements can be performed in order to dampen the rapid response reaction in glyphosate-resistant plants. This would prevent recognition and signaling of the glyphosate contact with the leaf surface of glyphosate-resistant plants.

Compounds that may be effective in conjunction with glyphosate are compounds involved in regulating the pathogen response system such as salicylic acid (SA), jasmonic acid (JA), nitric acid (NO) and ROS regulating compounds. In some embodiments, one or more of these compounds, as well as inhibitors and scavengers for them, are used in combination with glyphosate treatments. Typical SA biosynthesis inhibitors such as diphenyleneiodonium chloride, paclobutrazol and cinnamic acid may be used (Hu and Zhong, 2008). To regulate the biosynthesis of JA diethydithiocarbamate, brassinosteroids and the CO2 uptake regulator ABA may be used (Nahar et al., 2013). NO and ROS can be regulated by L-NAME, 7-nitro indazole and ascorbic acid (Pfeiffer et al., 1996). If glyphosate is received by a modified (mutated) surface LRR receptor kinase, which is normally involved in pathogen response, kinase inhibitors and modifiers, or drugs that regulate the surface presentation of LRR receptors might modify and dampen the pathogen-response of the plant to glyphosate, thus restoring sensitivity to glyphosate. Such drugs can be Wortmannin, Brefeldin A, etc.

Unless otherwise stated, all recombinant DNA techniques are carried out according to standard protocols as described in Sambrook et al. (1989) Molecular Cloning: A Laboratory Manual, Second Edition, Cold Spring Harbour Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy published by BIOS Scientific Publications Ltd (UK) and Blackwell Scientific Publications, UK.

Specific embodiments are described herein. Many modifications and other embodiments of the disclosure set forth herein will come to mind to one skilled in the art to which the disclosure pertains having the benefit of the teachings presented in the foregoing descriptions and the associated drawings. Therefore, it is to be understood that the disclosure is not to be limited to the specific embodiments disclosed and that modifications and other embodiments and combinations of embodiments are intended to be included within the scope of the appended claims. Although specific terms are employed herein, they are used in a generic and descriptive sense only and not for purposes of limitation.

1. A method for identifying an herbicide-resistant plant, the method comprising:
determining one or more genes that are differentially expressed in an herbicide-resistant plant compared to an herbicide-sensitive plant of the same species;
amplifying a nucleic acid associated with the one or more genes present in a biological sample from a plant;
quantifying expression of the nucleic acid; and
determining that the plant is herbicide-resistant based on the quantification of the nucleic acid.

2. The method of claim 1, wherein the biological sample is from Ambrosia trifida.

3. The method of claim 1, wherein the herbicide is glyphosate.

4. The method of claim 1, wherein the expression of the nucleic acid is quantified using qRT PCR.

5. The method of claim 1, wherein the one or more genes have at least four-fold differential expression in the herbicide-resistant plant compared to the herbicide-sensitive plant prior to application of the herbicide.

6. The method of claim 1, wherein the one or more genes is associated with cDNA selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 48, SEQ ID NO: 49, SEQ ID NO: 50, SEQ ID NO: 51, SEQ ID NO: 52, SEQ ID NO: 53, and SEQ ID NO: 54.

7. A method for identifying an herbicide-resistant plant, the method comprising:
amplifying a nucleic acid sequence present in a biological sample from a plant using at least two primers, wherein said at least two primers recognize a gene that is differentially expressed in an herbicide-resistant plant compared to an herbicide-sensitive plant of the same species;
quantifying expression of the nucleic acid sequence in the biological sample; and
determining that the plant is herbicide resistant based on the quantification of the nucleic acid.

8. The method of claim 7, wherein the at least two primers are selected from the group consisting of: SEQ ID NO: 121 and SEQ ID NO: 122, SEQ ID NO: 123 and SEQ ID NO: 124, SEQ ID NO: 125 and SEQ ID NO: 126, SEQ ID NO: 127 and SEQ ID NO: 128, SEQ ID NO: 129 and SEQ ID NO: 130, SEQ ID NO: 131 and SEQ ID NO: 132, SEQ ID NO: 133 and SEQ ID NO: 134, SEQ ID NO: 135 and SEQ ID NO: 136, SEQ ID NO: 137 and SEQ ID NO: 138, SEQ ID NO: 139 and SEQ ID NO: 140, SEQ ID NO: 141 and SEQ ID NO: 142, SEQ ID NO: 143 and SEQ ID NO: 144, SEQ ID NO: 145 and SEQ ID NO: 146, SEQ ID NO: 147 and SEQ ID NO: 148, SEQ ID NO: 149 and SEQ ID NO: 150, SEQ ID NO: 151 and SEQ ID NO: 152, SEQ ID NO: 153 and SEQ ID NO: 154, SEQ ID NO: 155 and SEQ ID NO: 156, SEQ ID NO: 157 and SEQ ID NO: 158, SEQ ID NO: 159 and SEQ ID NO: 160, SEQ ID NO: 161 and SEQ ID NO: 162, SEQ ID NO: 163 and SEQ ID NO: 164, SEQ ID NO: 165 and SEQ ID NO: 166, and SEQ ID NO: 167 and SEQ ID NO: 168.

9. The method of claim 7, wherein the at least two primers are selected from the group consisting of: SEQ ID NO: 169 and SEQ ID NO: 170, SEQ ID NO: 171 and SEQ ID NO: 172, SEQ ID NO: 173 and SEQ ID NO: 174, SEQ ID NO: 175 and SEQ ID NO: 176, SEQ ID NO: 177 and SEQ ID NO: 178, SEQ ID NO: 179 and SEQ ID NO: 180, SEQ ID NO: 181 and SEQ ID NO: 182, SEQ ID NO: 183 and SEQ ID NO: 184, SEQ ID NO: 185 and SEQ ID NO: 186, SEQ ID NO: 187 and SEQ ID NO: 188, SEQ ID NO: 189 and SEQ ID NO: 190, SEQ ID NO: 191 and SEQ ID NO: 192, SEQ ID NO: 193 and SEQ ID NO: 194, SEQ ID NO: 195 and SEQ ID NO: 196, SEQ ID NO: 197 and SEQ ID NO: 198, SEQ ID NO: 199 and SEQ ID NO: 200, SEQ ID NO: 201 and SEQ ID NO: 202, SEQ ID NO: 203 and SEQ ID NO: 204, SEQ ID NO: 205 and SEQ ID NO: 206, SEQ ID NO: 207 and SEQ ID NO: 208, and SEQ ID NO: 209 and SEQ ID NO: 210.

10. The method of claim 7, wherein the biological sample is from Ambrosia trifida and the herbicide is glyphosate.

11. A kit for identifying an herbicide-resistant plant, the kit comprising at least two primers, wherein said at least two primers recognize a gene that is differentially expressed in an herbicide-resistant plant compared to an herbicide-sensitive plant of the same species.

12. The kit of claim 11, further comprising at least one of a positive control and a negative control.

13. The kit of claim 11, wherein the gene is associated with cDNA selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 48, SEQ ID NO: 49, SEQ ID NO: 50, SEQ ID NO: 51, SEQ ID NO: 52, SEQ ID NO: 53, and SEQ ID NO: 54.

14. The kit of claim 11, further comprising components of a qRT PCR solution for amplifying a sequence associated with the gene.

15. The kit of claim 11, wherein the plant is Ambrosia trifida and the herbicide is glyphosate.

16. A specific probe for the identification of an herbicide-resistant plant, the specific probe comprising:
a nucleic acid that recognizes a part of a sequence of a gene of interest, wherein the gene of interest is differentially expressed in an herbicide-resistant plant compared to an herbicide-sensitive plant of the same species; and
a detectable marker linked to the nucleic acid.

17. The specific probe of claim 16, wherein the detectable marker is selected from the group consisting of a radioactive marker and a fluorescent marker.

18. A primer pair comprising a first primer capable of recognizing a first portion of a gene of interest and a second primer capable of recognizing a second portion of a gene of interest, wherein the gene of interest is differentially expressed in an herbicide-resistant plant compared to an herbicide-sensitive plant of the same species.

19. The primer pair of claim 18, wherein the primer pair is selected from the group consisting of: SEQ ID NO: 121 and SEQ ID NO: 122, SEQ ID NO: 123 and SEQ ID NO: 124, SEQ ID NO: 125 and SEQ ID NO: 126, SEQ ID NO: 127 and SEQ ID NO: 128, SEQ ID NO: 129 and SEQ ID NO: 130, SEQ ID NO: 131 and SEQ ID NO: 132, SEQ ID NO: 133 and SEQ ID NO: 134, SEQ ID NO: 135 and SEQ ID NO: 136, SEQ ID NO: 137 and SEQ ID NO: 138, SEQ ID NO: 139 and SEQ ID NO: 140, SEQ ID NO: 141 and SEQ ID NO: 142, SEQ ID NO: 143 and SEQ ID NO: 144, SEQ ID NO: 145 and SEQ ID NO: 146, SEQ ID NO: 147 and SEQ ID NO: 148, SEQ ID NO: 149 and SEQ ID NO: 150, SEQ ID NO: 151 and SEQ ID NO: 152, SEQ ID NO: 153 and SEQ ID NO: 154, SEQ ID NO: 155 and SEQ ID NO: 156, SEQ ID NO: 157 and SEQ ID NO: 158, SEQ ID NO: 159 and SEQ ID NO: 160, SEQ ID NO: 161 and SEQ ID NO: 162, SEQ ID NO: 163 and SEQ ID NO: 164, SEQ ID NO: 165 and SEQ ID NO: 166, and SEQ ID NO: 167 and SEQ ID NO: 168.

20. The primer pair of claim 18, wherein the primer pair is selected from the group consisting of: SEQ ID NO: 169 and SEQ ID NO: 170, SEQ ID NO: 171 and SEQ ID NO: 172, SEQ ID NO: 173 and SEQ ID NO: 174, SEQ ID NO: 175 and SEQ ID NO: 176, SEQ ID NO: 177 and SEQ ID NO: 178, SEQ ID NO: 179 and SEQ ID NO: 180, SEQ ID NO: 181 and SEQ ID NO: 182, SEQ ID NO: 183 and SEQ ID NO: 184, SEQ ID NO: 185 and SEQ ID NO: 186, SEQ ID NO: 187 and SEQ ID NO: 188, SEQ ID NO: 189 and SEQ ID NO: 190, SEQ ID NO: 191 and SEQ ID NO: 192, SEQ ID NO: 193 and SEQ ID NO: 194, SEQ ID NO: 195 and SEQ ID NO: 196, SEQ ID NO: 197 and SEQ ID NO: 198, SEQ ID NO: 199 and SEQ ID NO: 200, SEQ ID NO: 201 and SEQ ID NO: 202, SEQ ID NO: 203 and SEQ ID NO: 204, SEQ ID NO: 205 and SEQ ID NO: 206, SEQ ID NO: 207 and SEQ ID NO: 208, and SEQ ID NO: 209 and SEQ ID NO: 210.


SEQUENCE LISTING (210 sequences)

SEQ ID NO: 1 - DNA, 1247 bp, Artificial Sequence
  Synthetic Construct
gaatccaaacactcttcgagcccccgggcacaaactgaacgtcatggcattaaatctcctactactcggcatactcattagcttcttcgtttccacagatgcacaatctgtaggtgtgtgctatggagaaatgggcggaggtttgccatcccaacaagatgttgtaaacctttacagaaggagcggcataaccaggatgcgagtctacggtccgaatccaaacactcttcgagccctcggtggaaccaatatcgaactcattcttgatgttcctaacggttctcttgaggaactcactaatcaaaataatgcaaacatttgggtcagaaacaacgtagtaaactaccctaatgttagattcaggtacatctctgtcggaaatgaagtcgatccaaataacgtggacactagaaggttcgtcaatttcgtttttccagctatgagaaatgtccaaaacgcacttagagccgcaggcctagctatcaaggtctccaccgcaacctacacaggcctcctaaagaattcctatccaccaagtgatggtgtatttcatgataatgtgatcaactttattggaccaataatcacatttctagctcaaaatggttcaccaatgcttgccaacatctacccatactttgctcatattaacaaccctaaagatataaaactcccatatgcattatttaccgaacaaccacaaggtcaacagtactttaacctttttgatgccatgtatgatgctcattatgcagcccaatcgcgtcttggtggagccaatgttccaattgttgtgtcggagagtggatggccgtcagctggaagagatgaagcgacacttggaaacgctggaacttactatagaaacttgatttcacacgtcaaaagtggtagagggactcccgcaaggcctggaagactcatagagacatatttgtttgcaatgtttgatgaaaacaataaaccaggagcagagacagagaagcattttggggttttctctcctgatcaaacatctaagtatcaactaagcttttagctcaactaagcttttagctacataaacttgtaattttgctattaataataaataagaaatggaataataaaagttttaatttataaggaagaaactcgtgtctaccaattatgtcaattactcttgcatcatatcaaaatactagaaggaaaagaatgtatggtttaatatccctaatggagtagagttttagtatctaaaaaaaaaa

SEQ ID NO: 2 - DNA, 1628 bp, Artificial Sequence
  Synthetic Construct
ctccacgtagctgagataggctctgattcatcgccagcggttatctttcttcacggatttccggagatatggtacacttggcgccatcaaatgattgcggtcgcaaacgcaggtttcagagtaattgcacccgattacagaggatacggactatccgacatccctgcagaaccccataaggcctcattcactgatctggtcattgatactgcttcaatccttgagtctctatccatttccaaggtgttcgtgatcggtaaagattttggagcaatggttggctacacgttcgcactttttcaccctgaaaaagttgcaggaattataacacttggggtgccatttatgccccctggctctcaccatggttatctatctctccctgaaggtttctacatgagtagatggcgggaagagggaagagctgaagctgattttgcacggtttgatgctaaaactgttgtgaggaacatttacattttgttctctaaaggtgaaataccaatagccaacgaaaaccaagagataatggatttagtcgacccaacgactcctctacccacatggttcactgaggatgaccttgcagtgtatggagatttgtatcagaagtctggatttcaaactccactccaagttccttacaggtcactatcggatcaaagcgcgtatccgcaacatgatttgaaagtcgaagctccagcacttcttatcgtaggcgaagaagattacttctctaagttccctggaatggaggcatacttgaagagtggggaggccaagaagtttgtgccaaatctggaaatcgtgtatctgcctaaaggttgtcattttgttcatgaacaattcccggatcaagttaatgaactcatacttggttatctcaaccgcaacaaaaattagtgctgcatacacccttatttgtgatatggaaaataaaccttgagtgtcaaataaatgcgttagcatattacttcatgtataacggctaatattatgttatccgagtgtaatggtttgctttgttttgaggatgattatcattgacaattgcctattattagtgtttatgtgtttgtaaatgtgatgggaaaataagagggctacagtgtcgtcgatcatttgggtagaggatatgggtgcgggtcgaggcttcgaaatcctcatcttcataaacaaccctatataacccactatcatacgaaacaatcttcccaagaaacacaccactttcaaactccttcttcacatactgcccaacaagcaccacagacctcatcgcaagcaccgtcttccttgccggtttatccgctgttttatccgcatcttccccatccttcctcttccttttcctccccttactcttccccaccgccggttccatacccccaccccttctgatcaatccaaaacacgcttcccgtaaaccctagctccaacgcatcaatccacaatccagtcaaaacctcgtcggaaaacgttatccgccgttaagagactccgttagggtttcgtgtgcttcgaagatgatgaatatgtaaatgtgtgtgtgcgtgtgttatctgctctggtttgtacgaagatagattatagccctagagaggaaatgaataataatattaataata

SEQ ID NO: 3 - DNA, 1056 bp, Artificial Sequence
  Synthetic Construct
aagtattttgtgtaagtatataaaattgtaaagatgattgatgattaaccctcattttaacaggttggttaagtctatctaatttacaaatcttaaagatgttcggaccgcatttaccaaagtacaaatatattacacatgtttggaccagccaattaatatgataaagagttaacatagatattgtacaaaaaacacacacaaatttggacatatggtttttttatttttgaatcacgataatacaaaccctagtcacattatgcaaggattaaagtacaagagaaggcatgaccaacctcttcaaatcaatagctttgaaattaaacactcgtatctaagatgaaaacacgcacacaaacatatatagaaacacataaaacaaaacacaatcaacttttgagaaccatgccaaacaaatattgtagatctctatgcgcattggaaaccctttggcaccttctttccacaatagtttagcaacaaactcaatgaaactggcacattaaggttgatacccaatacattagctttgatcgcggtgcaaagacaaactgcggcctcaagatcaacaaggtcaccgaggagactgcaacaaggagtcttcgcgggtgtaccaacaactaggtgaaccaagtcattaaggacattagcacacacacctagcttgagggtgtctttagggcaagtggctttctggtggtggtgtttatgtggcttaggggcctttggtggtggtgggcaagaggttgagctcacaagagtgaagaagagtaggttgagggtgagtaggaaggcggttgttgccaaagaccttgaagccattttggatgtcttttgggttgtgaagggaaaactagatcaagttatggttactactttctagttttggggtgaggaactattgaagtgtgtcatgatatttatagatgagaatgatgcacagtgttatgcattattgacatgtcttcatgctatgttaggtagtttaattatatagtcagcatcaatcttttactcacttggttatgtaactagagcattatcataactttt

SEQ ID NO: 4 - DNA, 1140 bp, Artificial Sequence
  Synthetic Construct
ttttttttgatgatgagattatgttactttttgtattaatatataatatgctggtttgttcaaaaaaaaaataaaaaaatagaacaactaatcttaaatcccaactacaaaaccatttatatcacacacatgcataaatttatgcatttacaagctaattttacaaagctgtagcttactgagagaagaattgtgtacaatcatcgagaccatagatggcacaaacggcattctccgaattaattaaacaccattatcttctactttaaaccaacattcatcattttgatattttggccactaatatatccactgaattttataggaaaataacttgttttcaagcttcaattcatcttacccaaatttagccgtgccttatttgtttcgtttatacctcattgtcttttgattgcgctctttcacttacctcataaatttattcaccttcccccaatgaattatttaccttccatgtaacatgtcgatttattgacgaatcttaatatgttttaacgatatatgtatgcttaaccgcaaaacacaataaaaattatagaactcttgtacatcatatatatatataagttatcatatatagttacaagtacagaaaattaacaagatactagtgttttggtcttagttcataagtggagaactatgacattattgacgtggcataatatgcatcactgctaaacttttattttcttttctaacttcacctcctcactggatcctgttacagtctgtggaagggctaatcttgtagggcaagttcacaccacattttccggggagactagctgcgtaattgccgctaacgccgcggtaagacgatgaagcactcttgagacaattacaaatggtcttacggtctgcagtagtacgtgcctgattcctaagtcctctaatcccattacaacactgtgctggcggagccccaccattccttaggtacccgaggcacggtgctacagaacccacaacctgaccacaagtcactgcctccgcctcgggcacggttaggaacgccaccagcaacaccaccatgcatgaggccttgatgaacatttttgtcattgctacaagtgggaattgatgggagttaatgtaaatgtgaatgctttgaatgtc

SEQ ID NO: 5 - DNA, 1877 bp, Artificial Sequence
  Synthetic Construct
caagcaatggagttgacgtgaaggttgttgagagaccaaatgttgcaagtgaccatccacacaattctcgaagcataagcagaagtcctcaaagggccttgagcaagagcatgagtattagtcctagggggagcccaagtgtgagcccaaggcgcaataagagcaggagtccgagtgtcagtgggtcccctagacgtgataccttgcaaaaaacccggagcccagttagaagtgaaagcagcaagagtccagctagaagcattagcagaagtccaatgacaggcaaaaacatcagaagcctaagcccgagtccggttagaccccggatccgaaaaagtgtgagcaagagtcctacactgtcaccatcgccgcctcgaagtagaaaatctttaccgaagtctgtgagcagaagtcctgttagatcttctagaagaagttatagcagaagcccggtaagatcctctaaacgaagcttaagcagaagcccggtgagatcctctaaacgaagcataagtagaagctcaggtcgagctccattgaggagaagcctgagtcgtagccctatcaggccacctgttaggggcagtcggcgtagttattcccgaagccctgttggggggcgcaggggcaggtcaccttctagaagtgtttctcctgatgggtcccctaaacgtatcagaagaggtaggggtttcagtaatcgatattcttatgcacgtagataccattctccagatcgttcaccggtcagatcttatcgctacggcaggagcgaccgtgacaggtattcaagctataggcgtcgcagccccttgagggacagaagtccaccaaggtacagagctagaagaagcaggacccgaagcccaagtgtatcacgaagccctgttcggtaccgccgccgttacagccgcagccgctctcctgtcgagacatctcgatacaggccttcaccaccgattgaaaggagaagagcgccacgtagcataaccccaccttctagaagcaggagcccttcacgttcaccatctccaagaaggacagataggtcgagatcaaagtcaaagtcaaaatcgatatcaaagtcgaggtcgaggtcgaggtctaggtcatcttctggaagcccgcctgctaagggaggactagtatcctatggagacgggtctccagactctggtccgaagtgatgggttttgatggtgcttgagttatatttgatgataaatagacttgagaggctgtagggtgtgttttgaataacagacagacagacagacagtgttccttaaggatggatgttgtgacgacttctgccatcgccccccaaaagcatctctcacagtgggacttcagctgtgatttggaagtagattacaaatctgaggaaaatgcttccatcgtatattcagcactggctattgacaaggagcttcagccagataaagttaggcgggtgatgtctgtctcaaacggaaaactttcagtacggtttgaagcaactgaagctagatttctccgtgcatcttacagcgcgtttgtggatgttctaacactagctactaaaacaattgagcaatttggtaaaggcatggaagtataacatgggtaatcatgatcattaaactcaaaaccatttgatgtatgtggaatttattttcttaattttataagcataggatattgttcgtggacacatgttctgtagatgcatatttgtttgtttactagcagttataatcatttgtatcagttggttaaatgtcttaaatgtaatgctgtcaaattttatgcatgccactatatattattatcttcaaaggtgtatttcttccaaacaatggttcaattcaatctcatgtaatgtatggcaatgatgt

SEQ ID NO: 6 - DNA, 1094 bp, Artificial Sequence
  Synthetic Construct
gtgatatcgacccgaaatgcagcacaaaccttaaacaaaccgaattcttcgacaacacaaaccttaaattactctactagtgttttgttctcgaagattctttagcttcacatttcaaaccaatcttttatggctcgatctatctcgaacgctaagctcatttcatcgttcattaccgatcaaatcgccattgtttctaggcgtggatatgctgtgatatcgacccgaaatgcagctgcaatgatgaagaaaggaggtgaggaatcgaaaggatcgaattcatgggttcctgatccagtaaccggttactataaacccgagggccaaactaaccaaatcgatgcagctgatctccgtcatcagcttttgaaacaaaagaatcgtcgacaatgagttcattaggataatcgaatatgaaattaaaccgatgaacgatgattagatgatgaattttgattatttggctactatactatggcatatcaatctatatatttgttcgtgtgtattgttcttactaggtaccaataattctacttatgaaatattaatatttgttagaaaaaataaaactcttgtttagagtaaaaaatttaaaacgttaatctcagtcagtggtaccccttgactttcttggcgtagtacatttttagtgtaaaaactgtgtttcttctttgttttatgtatcggctacccttgcctaggttttttttaacccgcgccacccctcaacttttcttctagattcatcactaataacatgtagggtatgaagaaggttatgaagtctcttgtagtttgtatttttgtaacctaattagttgttgtacataaatgaaattaaaacaaaatatattgagagaaaaaaataagttttggtcatgtgtttgcatctacgtccctaccatcaaggtaggtacatgaacccaacttaaaatataattttttgggtaacgtctaaatatatatttttgatacgtatgttatttaggagtgaaatatatgcttttggtgtagcaatcggatacgcaacatgtgctagcttttgatatttgttattttttatatatagatgggtgaactaaagaagctgg

SEQ ID NO: 7 - DNA, 1277 bp, Artificial Sequence
  Synthetic Construct
aatttaattcttttttttttggtcactattaattacttttatccttaggcatccacctttaattatcatcaggagatcgatcattaacttagtcattattaattaattagggtatggagttgttacccaaaccatcaatacatagatacatagatagataatacattaacacctatatatgatgaaattaatcaatatactagctcccaacacaagcaattttttattgaacacacacacacacatacagattaaccgatactgttggtggtctaataattaagcggttgcaatgtccatgctcttcataacttcaaagcgaggctccttaggctgaaatttcacttgagcatacaagttcatgtagtcctcaaacacgaatttggggtactgcatatttttgttattaaccagttgtggcgcaggatagatgagtgcgtcactccctgggttgtagaaggaggctatggacatgcggccagtgccgtgagtctgagcaatgactctgtgcagcacgctcttgtaccttccattggtgattacctctagctgatcacccaagttaatgacaatggagtggtgcatgggcggcacatccacccacttgtcatccttaagcagctggaggccactcaccttatcgtcctggaagagcaagatgacgcccccagcgtccgtgtgggccctcagacccttgatcaggtctggcttgggacacggagggtagttgctcaccttggtcccgaaggtggggcccttggagttggcgccatagaatgccttctttaggtaacccttgtccagacccagattttcacataggatgtccaaaatatcctcagctaggagttgaagctgcttagcgaattccctcatgaggtccctgtagtcgggttgaagatcggggatgtcagagatgttggattgggggagatggcggagatagaaggtgctctcccagtccaggtcatggatttcagtacgaagctgggcctcatcagctaagcctttggcggccaccatttgtttgaagcgctcctccatgcacttcttgtaatgctcctttgtcatcttctccactctgtccaaaagctcagtggagatcccatgattcacaatctgaaagaatccccaattttcacaagcatcattgattaggttcattgttgcagctctttcttctccgttcagcttctccatgttgacaactggaaatgtctccatgactcctcgatctctagcttgagtgtgtatgtgtgtgtgtgtgtgtgtgtgtg

SEQ ID NO: 8 - DNA, 3263 bp, Artificial Sequence
  Synthetic Construct
gcggttaggactccaggtctaaacgcaccggttacatcacgcagtaattgcaacctgctgtcagtcacaccttcttctctcatttcactaggcatatcgacataataattcatgtcgttgaatgacatggcgagtggagtaaacggaagaaccatccctttcttagacgcgccattagtattagaaatggaagaactaatctggcgcatttgcttattccttgaatcgtctacttcagcagcatgaccaccgttcatggctgcagcttcggcctttgagatgatcgcttgtggttttccaggcgcttctaaatacataagcgacacggtaaataggatgttgaagagaactataaaaccaagaagcgcaccaacgccaatccagtatgagctttctgaagtcgggacgtccaaactctttaacacagcaaatcctaagttggttgtaccgtcggtactattttggctggtccatctagagtcaaggaactcattgatggtgatggatttgaagatgtaagacaagggagatacccaataagcccattcccaccagttcggaagttgagttttcggtaggatgaaaccacccaacaaaaatataagcagaagtacaacggatccacccgtgttagccatagacatcgtcttacaaacgcctgcaatcagcctaaacagtcccgctgccacgttctggatcaggaaaaccaacaggagatgcttgaagaacctgtcagcagtaggggcaaagtcaacaccccagtacagaagaacagtccacataatactttcgatcatagacatgggaattcgaagcaagaaagccggtaacgtgaaagcccacggagggtggaacatcaggtcccgctgtttgtaaacgacaggaagtctcatcacagtgaacgacaaatcagcaagcccattaaacatgttaataagcatactgtctaacagtgcaccgacgtaaagggatccgtctatctcgttccttgtgtgcattgtggtccttagatacaaagttgtgcaaataaacgatataaacacaatttgtatagttctgaaaacatagacgggagcgtttcttttcatcagcaatatttctttatcccatgaggccttaagaagctccatttttggtaccatgtacttcttaaacactaaagcagccttatggctttggttcttgtcgtatgggactgataactcgcgatgcagcttcgcccctacatgaaaattcttgaaccgttgactaaactcgctcaccgggatgtatctgtatgctctgtttctatctgcccaatattgctcttggtcctttttagatgtcacctcttgtaagaagtcagcagtgccctttctctccggacatttgaatccacaactctcgaagaactcaacaatgtgttctcgtgggcctagatacacaatctggccctcggaaagtaggatgatgtcatcaaaaaggtcaaaggtctccggggcaggttgtagtagcgacattaagattgtattgtccgagaaatgtgccacttgttgcatgcattttactatctgaaatgtagtcgagctgtctagacctgttgatatttcgtccataaacagcgtttttgctggtccgacaatcatttctcctgtggtcacacgcttcttctgtcctccggagatacctcgtctcatttggtctccgacaaaggtgtcacgacagacatcaagtcccaaaattctgagggtgtagtaagtgatcaaactgctctcatctccttcaatggcggtcgccttcatgaaaagatcaacttcggcttccgggaagatccctgcttccttttctcttcttgcaagctcagttaacatttctaaacgagatccaactccttggcatcttgctgaaaaatccagcgtttctttaaccgtcatttctcctgcatgaatatccatctggctaatgtaagctgctgtccttcgtggttcgaattcattgagcttatgaccattgtaagttatctctccatcaactcttaaagcaggatccagccttcccgccaaagccaacagaagcgtcgtcttaccagaagacggtggccccaacagaagtgtcatccttccaggtttgatcattcctgtggtatctttaagaatgccgagttttgccttctcggcgaaactgataccaacagaagctagaagagattcaacaatatttctggctgcgtttatcagcgttggcagcgctctatctcctacgtaacaatcggcttctacagacagattttgaaaccgaacttctactgttggtagggagattccaaccttatcaacacgatctctaaacttcctcaaaaaaatctcattatcctcttcagcaaccttaaaaatcttatcaataaacccttgtcttgcctgaggatcaagctctcgaacatctactaacatctgatcagtaggctggtgatcagcaggaacatacgacttaaatatagtcgttcgtaaccggttatacgtaggcagtttttccaaagcagcccatcgtaacgcctcttcgtcttccatcgagtgtcgactactccgaccatcgtgactaccaccaccacccatcgcaaacgcgtcatccatacgccagctgcctgaagctctactaatccctctgcttatgcttcttcctaaactctctgtaatactcatttttttattttcatccattttttttaaacaaacaaattaatatgaatatgtttagttcatggatttcttgaactttatatacagagacgtctcctctgttgctagtataagaaaacatctaaatcagaaatccacctggcgaggagggggcatgatgtcataacacaaaacagctaaaggatatatatggcccggcacaccagcacgctccacaagtcttctcatgttgtccacagcagaggcatcaaatggtgccggagaccactgatttgtgatgggatctggccggtccctgccgccacttgctgcaatccatatcagcttagatccacccctcaaaagcaaagccatcttctttaaagttattgtatttgctctccttttcatctcagcaagctcaggaacgtcattcatatgtttctttgaatacacacataacagattcctgcccatactgaaaggcttgcaaagaggatctgtaagaactctatcacctgccacatagattgcggtctcagaaatatgaggacttgtggtttcaagcagcaaagagatgataactggatcagcttccgtttggtggtt

SEQ ID NO: 9 - DNA, 2259 bp, Artificial Sequence
  Synthetic Construct
aaatttgccctactgagcaagaaaatagaagggccgggacacccctgaccctttatatccaccgccaatgaatgtagtcaaagttgggttgtaaaatgctaatataatgtaatgtgatatgatgtaggacattgtgagccccatattatttgtaatcctaaatggaacaaaattattcttaacatatatgatttacaaacaaatactgggcacataacaacttaattaagtccttacattaacaagagggatacatcatttgaacactaaatgcaaacaaacaaaaatatattagaaaatacaaattaaacacttacacaaattcacactaacatatgggaaggggtgcaccattccatccttcaacacactccaacaatggatcgattattagaccttgtgacatggccgtgaacaccttgtcgaactcctccccgggcgaagtcaccttctcgccggtcaagtatgcgccgctaagctcctccctcacaaaccgatacaatgggtacgacctacacgccttgatcctgttgggaattgacattgtaccattctcaaatgcgactctaacactctcaacttccttaggtaaaatggccttcaactcgtcttcaaaagtcgcaatcttttggaagatagacgtgctagtgttcttctcggtttccccattgtttaaggcgtggtcgacaaggacttggcggagcttttgcattaatggatacgtggcgaggcaagggtcatcaatgtaggcgaaaacgtactcacggtcaacaacacggagcaagtctttttcacaaaacctcgatgggtggagctcgccgtttacgcccatggttaggacctttttcgctacttggctcacggtgttcttcacggttgatttcatgttctcttccaaatgcctcaagtcaatagattggcataatgcgactaagtacgttgatgacataagctttaaaatgtcgaccgcttccgcggttttcctcgcagaaatcaaccctaaagagttaacatcttggttatgttgttccgcgctttggacatggtttgtaaccggattcgctagaaactgaagctccgaacagtaagaagccatcgcgatctccccgcctttgaacccgtaatccaaacttgggttgcgcccacccgaaagatttgaaggcaacccattgttgtaaaaatcattcacaagctcggaaaattgggcaaacatgagttttcctatggctgcaatagccaaacgtgtgttgtccattgagacaccaattggggttccttgaaagttgccaccgtgtaaggctttgtttctcgaaacatcgatcaacgggttgtcgttaacggagttaatctcacgttcgatcattttggtggcggaccgaatgacttcgatttgtgggcccaaccattgtggggaagtgcggagcgcgtaacggtcttgtttgggcttttgtaacgggtccatttcatggaccttttgggccgctttaacataatcacttccatccaaaatgtactccatgatggccgcggcctctatttggcccggatggtgcttcaatttatgggtcaagtggtccgtaaactcaggcttcccttgcataacttccgcgaaaatcgcggacaacacttccgacaacaacgcaagtacattagcttcaaacaacaccatggacgccatcccggatcctaccgcggtaccattaacgagcgccaacccttctttcggctgtaactcgaagaatccgccttccacgccagctttggcgaaggcggattccgcattgagggtttcaccggccgggccgaccgctttggagttgggccggccggtgaggaggcccgcgatgtaggatagcgggacgaggtcccccgaggcggtgatggtgccgcggaggggcaaacacggggtgatgttgttgttgaggaacttagttatggcttccaagatctcgaatcgaatgccggagtaaccctggaggagggtgttgattcggacgagcatggcggccctggtggcggaatgcggcagtgtgtggctagattctgtaccgttgccgaatattccggcgttcaggaatctaataagctccttttgtagagcaccaccattcttagtcctcctatgagacgtagcaccaaatccagtagtaacaccataactatccgtccctttgttcatgctctccataacccaatcactactcgccttaaccccggcccgagccgcctccgaaagctctactttcaccatgttgtcac

SEQ ID NO: 10 - DNA, 497 bp, Artificial Sequence
  Synthetic Construct
ccaaaccgcattgatggagagtggattcatgttacccgatcctaaggacttcgcttcacgtatctacgattccgtgaaatccagtttgagcattagccccgatgcggctgttgaggaggaagatgaagtggaagaagcccctgaggttgagacaaccagcaccgagaagggagaagctgattctacacccacagaagaagagcatgaggatattaaggatgagttataggtctgttagcgagttaactcgttaggtttaaacaaagttttattgtcggaagatgatatgcattcgtgtactgtttttgccattggcttggaaaatgtactacatacgttgttagcaggagttgtgtgttttgagtaattttccacctttgttaattagattacttcttgtaccttttttagatttgagttcaaaacaattatcgagtaagactgttaaagataaaccagtgaaccgttaacgaacacaactacaagcattcgaattg

SEQ ID NO: 11 - DNA, 900 bp, Artificial Sequence
  Synthetic Construct
agtggaaccaccaccaccaacttcttccccttctctctctaaaaccctctctctctctctctctaaactctctctcatggcaaccaccaccaccaccgtcaccgtcctcttaataaccatcatcctctccatctccaccatcaccaccaccgccagaccctgcaaaaccatcttcttcatcacctcaaccaccaccacctcctatttccccaccaatcaccctctccaaaaccctaaccctaaccctaaccctcgtcttaccttcttcatcaccgaaatccaccacttccaccgcacccgtcccttatcccaccacgctttcacccatcactcctcccaaaacgatgtcgtttcatccaaaccctatttcccttcctcttcaccttcgattcgtgaccgtaccatggatattatgagcattgtcggtgccttattgttcggtgtaggctgtggtgctctcactgctggaaccatctacttggtctggtctttgttttctaacaaccgtgcgattgattttgattccgatgattctgacgttgaaagtgttgatgatgacgtcacccctaacaaaaacggttatgttgctgttgctaataaggaagttcctccttctgctgatgaggttgatgccatgaagaagtaataataataattgatggtgttttgttggtaatctgtcgattcttgtttttctgttactgtgatgtgtatttgataatatactgtgaattgcaaggtttattgtgatttttatggttgttatgctatatgtctgtgtgtgtgattgtaaggctcctactgtgttttataaaagttaactttctaatggtttgcggtatttgtgattttaggtagaaattagtgttctgtttgaatttgttctaaattggtatggat

SEQ ID NO: 12 - DNA, 517 bp, Artificial Sequence
  Synthetic Construct
ctttgaccgacttcctcgtgcccacttgttcgcgtgacccgacacgtcgtcttgaacgacgtgttaacagaatcggaacgttgaccaggtctgactcatgaccgaattttgaccaggagttgaccggacccgaccctggcccgaacatccaaacgaaaccgaaagaaccagatctgacccggatctaacctggaccaggtatgactccgaggtctgaaagctaaccagatctccgagtccggaactcgtaaacgaacgcaagcggcccgatccaaaacctgactgaaccacaaataaaccagcgccacatctggccaataaccagccgttagccaccactggcccgagtccaaccaccgtgaaccatctctaggacattaccatcatcccttgtcagcctttggtcgaccaagacagcaccaccacatggtgtaagaccaccatcggccaccaacaaaagaaaatcaactaccgatcgtcaccacaaacccacaaccataaccagaccaaacgaacc

SEQ ID NO: 13 - DNA, 2029 bp, Artificial Sequence
  Synthetic Construct
ttttttttgaaccaagaataattcaataaattttatttcatgatgttattcatagcacaaatttgcatagatttccattgtatgacatttaatttaacaattaatagtatttcaatttattagtaccatttattagtaccatgtgattgtgaatagttattctcacatgataccctgatctttgtgcaatcagttgggtattacaataacaaagattaagacaaacattaagaaagagtaatcctaatatgcagacatgatcacaagtctaaacatatgatatgtttatggtgagtgcaaattttaacaaagtgagttgtaccaccagacaaaatcatatcaacgtagttaatgatctattagcggctttgctgatatgaaatgctggaccagtgcaagagccctctttggttggtagcttggcacttggtgaccagcctctcggactgtcactaatgttatatctcctttataaacttgtgcaaagccagccgcctctctttgatgaaaccaaggatgccatttagtttttataggaagcttcatggaactaatggaatacttggttgatgtgattggtactcttgcatcagtatctccactaaatatccaaacgcgaagcttgagtttcatgaattcctttagaagtggaataactgtcaaggagctatctgtccaagttgtgaggactcgactgcatgactcccagtcatgatcgagcttcgttacattggcatggagggcctcttgaacatctcgacggttcatataagcatgggtgtagtaatcactacaagggtcgaaattttgccacgatgtcttcttaggatttgtagtaagattgccatcaaagcataaaggggcatagatgttatacatgtcaaccgcgttagtgttgtaccaaatatcttttgcagcgtcaatgcacatatccgattgtgtgaccgcttttggtgaaaagttgcaatatttatgaatcgcgaaagtagtttcatctgatattatagcatgtgaaccaaggtaatcgatcatccctatcaaatctgtctcgtcgttgattactgcattcccaataaggatccctttgagattgatgagggttttattatgcttcatgttgtagtaaataatggtttgggcgagctgaggcacataatgtcctgcatagctctctcccgctatataaaaatccctatctttatactctggaaatcgttcaagccaattcaataaaaatacataattatctgccgctgtagatttgtcacccatactttgataatcagaggatctgttagagtatgaaaaccccactccagccggcgactctacaaataatacattcgcagcacgattccaagaaaatttgttttcataaagagtttttccatcgcttttgattcggaatggtccgagctcctgcattgctccataagcaagcgatgagcaacctggccctccatttagccaaagaagaagaggcaaagactccttagatttctcagcttcaacaaagtaataataaaatgccctgccagcggattcacttacggtgacgtagccaccatattggtcaaacttaacatatggttggccgggaagtttgtcgattttatcattctcttttctccccgtttgaggaagaacgacttttgctttgtgtaaacctttaggtgtctcgaaaaggcttatgtttaccggagaggcttttgatagcttcgctttgtagagtcgagccaatgcatggagttgtttgttggcgttagtttgatatacgaatgttgaaaggaatagcgagatgaaaatagccaaaatagttttattcttcattattgtatctctagagcgagatggtttgagtaatggtgttaaaggtttaggtacgtagtgtatagaatttatatggtatgggtgtggtacgcattttcttgtgaccttgtattacctacatgactgtctcatataattgagatgggtccactgcataacaaagctacaagtttattcaatcatataatttatcatcagg

SEQ ID NO: 14 - DNA, 1309 bp, Artificial Sequence
  Synthetic Construct
gcgaatccttgtctttgtcggtgcccgttgattacgcgtcgagacgcaacttccatcatatttctttcactttgtctttgtcagtaagtagctacgtgctactagtataaaaccaaacatttgttactccatttttacttcattgaaatctcccgttcaatttttcgatccggacttgcattttcggtcaagttaattttctaaagtaactgttcatctctaattaagcaattgaaggcatgatgagggtatgtataattttggtgggcttattaagccttttgagcattgaagcttatgaagttggactcacttttctggacagtgccgttgctcaaggagctgtttgtttggatggaagtgcccctgcatatcatatggacaagggattcggtgaaggaatcgacaactggttggttttctttgagggaggaggttggtgcaacaatgtcactaattgccttgcccgaagagatactcgtttaggttcctctaagcaaatgttaacaaccgagaccttctcagggatgttccacaacaagccaaaatataaccctgatttttatgattggaatagaattaaggttcggtattgtgatggagcctcttttaccggtgatgtggaagaagttgatcctaaaaccaatcttcactttagaggagcaaggattttccgtgctgttattgaagatttattagctaaaggaatgaagaacgcaaagaatgccatgcttgctggctgttcagctggtggacttacttccatactgcagtgtgataactttagatcgcttttacctgcaaccactaaagtgaaatgcctctccgacgctggttttttcattaacgcaaagacgattattggaacaagtcatattgaaggcttttatgcggatgttgttagcacgcatggttcggctaatgttttgtctccagcttgcacttcgaaaatgagtcctggcttgtgtttttttccacaaaacatggtgcaatatattaaaacaccaattttcttgattaatgctgcttatgattcatggcaggtaaagaatatattagcaccaggggttgctgatcgaaaaggcacatggcgtgaatgcaaacttgatataacaaagtgttcatcttctcaactcaatatcctccaaggatacagacaagagttcttaaaggcattgactggtttcgataattctccatcgagaggaatgttcatcaattcttgttattcccattgtcaaactggaactcaagaaacatggttaagaaatgattctccactgttgagcaacacgacgattgcaaaagc

SEQ ID NO: 15 - DNA, 1946 bp, Artificial Sequence
  Synthetic Construct
cagctgggatattataattaatagatctaatacacattgaatctctctctatatatacttgtattctccatcgacaaacaatcctatcttctctaaaaccttttatatatacgttctcttatttactatttcattaactcatcagagttgccattatggccggtggtggaataccaatcggtcccggcggtgataaagcttaccccgggaacctcactctttatgtcacctttacttgcgttgttgctgccatgggtggtcttattttcggttacgatatcggaatctccggtggagtgacggcaatggatccatttctaaagaagttcttcccatcggtttaccgaaaacaatcggcggatcatacgacaaaccagtattgtaagttcgacagtcaaacgttaaccatgttcacgtcgtccttgtatcttgcggcgttgttgtcgtcgttggttgcatcgaccgttacccggacacttggaaggaagctctccatgctttttggaggtgtcctgttctgtgccggtgctctgattaacggttttgctcaagctgtttggatgctcatcgttggtcgtattcttcttggatttggtattggatttgccaatcagtctgtgccactatatctttcggaaatggcaccatacaagtaccgagggtcacttaacattggctttcagttgtcaatcacaattggtatcttagtcgctaacgtattgaactacttctttaacaaacttgatggcaatttgggatggaggcttagtctagggggtgcggtggtccccgccttaattatcacggtggggtccctgatcctccctgaaacccctaactcaatgatcgagcgtggcaataaagaaaaggcgagatcaaagcttcgtagaatccgaggtgttgatgatattgatgaggaatttaatgatttgatttacgcgagtgaagagtcaaggaaaatcgaacatccatggaggaatttgttgcaaaagaaatataggccacaactaacaatggctatattaatcccattttttcaacaattgaccgggattaatgtgattatgttttatgccccggtgttgttcaaaactataggttttggcgatgatgcttcacttatgtcagctgtgattacgggtagcgtaaatgttttggccacatgtgtttcgatctatggtgttgataagtggggacgtagatttcttttcctcgaaggaggcactcaaatgctcatttgtcaggttgcggttgcaattttcattggaatcaaatttggagtaaacggagatccaggcgatttgcccaaatggtatgccatagtagttgtgcttttcatatgcatatacgtcgctggattcgcgtggtcgtggggtccacttggatggctcgtgcctagtgaaatctttcctcttgagatccgttcagctgctcagagtattaatgtttccgttaacatgatcttcacattcataatcgctcaagtgttcttaaccatgttatgccattttaagttcgggcttttcctattcttcgccttttgggttgttgtcatgacaatcttcgtgtactttttcttgcccgaaacaaagaacatcccgattgaagaaatggtgatcgtgtggaagaagcattggttttggtcaaaatttatggttgatgttgattacccgaatggccttgaactaagtaaaggcggtgatgtcgtaaagaaagtataacaaattcagtatcatgtcaattgtcatgttttagtgattttatatattgtgacgtattagtagtttgttttaaagaacctctccaaaaacaagagtttgatttttttaaatggtcaagtgaatataatggtttatcatatttgatttctgaataccgttctagttatacgatgattgaattggatattggtctaaaactgtatcttttttttttttttttttttt

SEQ ID NO: 16 - DNA, 571 bp, Artificial Sequence
  Synthetic Construct
aaaggctccaacaccttcaccacccaaagacacttgcccaatagacacactcaagttaggtgtatgtgctaacttactaaatgaccttatccacctagtcgtgggaaccccaccgaaaacaccatgttgcacactccttacgggacttgccgatcttgaggctgccgtctgcctttgcaccgcaatcaaagctaatatcttgggcatcaacctaaacatacccatctccttgagtctcttgattaactactgtggcaagaaggttcccactggcttccagtgtgcttagatagtttgtcttctagaattatggaaggttgaaactttaacttgtgtctttggaatatgtatttatatttggtcatgatcaatacttgtaaagaatcatagcgatatgattcaagaataaatttatgtcttctcttatattgttttatgatatatatgtatgtacggagtctaaaatgctacaaacatgtatgtggcatttgggattgtaattctaaagcaatgtctagaagtgagatttgaaattataataaataatttttttagtgatcgaaaaaaaaaa

SEQ ID NO: 17 - DNA, 335 bp, Artificial Sequence
  Synthetic Construct
aaacccgttttactaagaaaaaaagaaacccaatttgagacggaatgtgattgcttctctataaattgttccccaagacacactccattctcccatcccattaatattaatattattgctaacaaattcttcttccctcaataatcttcatcaccatgaccaaccagcctgaatcctcttcatccgatccgaaagcgtcgaagaaggacttttcgactgctatcttggagcgcaagaaatctcctaatcgccttgttgttgacgaagcggttaatgatgataactccgtcgtcgctatgcaccctgccaccatggaaaaattgcagcttttccgt

SEQ ID NO: 18 - DNA, 300 bp, Artificial Sequence
  Synthetic Construct
agagactcctagagaagaagcaggtaaacatgctcttcacattaaaattttttttcttagataaatattaccctcttataaattacacaaaattatcatgttgcttgagaatgaatctaagacctcccactaagagaaaagagtctctatcaagttgacagttaaatatttaatcggcgtaaatcatataataacatacatgaaaagttaatttaagaggtgaaagtttgtattagtttattatactaaatattcttgtatctatttgatgtaatttagttgaaagatgactcaaaaggc

SEQ ID NO: 19 - DNA, 301 bp, Artificial Sequence
  Synthetic Construct
gtttccttttaagctaatgtctttctaaccgtttaacctgttaaagataatacataaaccgaatcaaccctttgtttactaaatgtgaatgggcaggcaaacaactgcaccccaaacaatattatagtacctgatttaaatctcaattaataattaattgaaaatgagtacaacacacttttgtcattatgctataactagtaagcagtaagcacacacttttctttgtagaatttctatagatcactgagaccccttaaaaaaatagaaaagtaaaaagaaaagataaaaaaaaaaagta

SEQ ID NO: 20 - DNA, 1755 bp, Artificial Sequence
  Synthetic Construct
gaagattttataataacttgtttaagtgatattttgaacttctattttcaaacaaaatgcttacagttccattttttaatagaacttgaacaattttataaaaccatttaatttgttgctacttatagctatcgcagaccgccccgtgtacccacacgggttatgctctggtgcgtgtagccaatgctatgatgaatgccgcattcgcgagcatgatgctaacaaatttaaacgttcagaatccattattattcattcaaattgaaataccaaactaacaaagtccgttttaaacttgcatccaaacggaagtcaaaacataagttaaaatggaacttaaaattagcaactgatgagggctttccgcatcgtcatcaatcacatccttgagatttggagaggaaacgcggcttcaaatatcgcatcactgaaagagagcaaacgaaacttgtccaagaaaccgcgtttttccataagaaggttccggcatttttctgcagcatctacaccggacgagtaagctccatgcaccgatccctgattctccatgctaaccgcttcacctccaaagaagagattacccaaaggagcacgtagcctgtcataaatatcgcttggctttccaaccacatcgtatgaatagcatccaagggagttcgggtctgttccccaacgcgaaacaagatactgaacaggttgggttgcttgagggaacatcttcttaagctgatccatcgcaaaatcagcagctgcttcctcggatagattctcgaggtcataagcaaatctcccagcagccatatagacaaggaccgggtgtcctgttgccttgtgtagattcagaaaatagccacaagaatacgacgttggagcaacggtccccagcagctcgacgtttggccagaacacgttatcgaaacgtagcgcaatcttgttttcattccccataccaagatctgatatagcagaggtcttccactctggtagttctggctcgaattcgatcaacttcgcctttagtaccccgatagggaccgtaacgatagcagcatctgcaacaaattgtcttcctccctcaactgtaaccaccacttccttagacccttcgtttgatatccttgtaaccctgtgattcaaacgaatatcaatatcctttgccagagcgtttagcacaggatagtaaccctcaaccatcagcccgtgcccaccagtgagaacgttttcctcatcccagtttttcagagatatcatatctaaatctgcagcaaaccatgcttccattctacatacgtaccattgtagcacttcatacgccagtccctcttgtcttaaatctggatgtcgttccaaaactactgatattgcctgaagaacggaaagatcgtgttgattttcattcctcacttcttcggtcttcttgagaattctcttgaatgcttctccgacttctataacgatctgttgagggacctgcttaccttccttgtcaaatagcatatagctctccaaatcgtggtcatataatacagagttgtcaccacttgtccgatataacgcaagccctaaatggcgaatcaatggggccaaaggattttcattgcagacaccatgtaaccatgatgctcccatatcgacggggcaaccaaacgaataatcagtgtgtatccggccaccgattctatcccgcgattccaataacacaacctcaaaatcagcattgttgagaacatgagccgcagatatccctgaaatacc

SEQ ID NO: 21 - DNA, 1702 bp, Artificial Sequence
  Synthetic Construct
gtggcagagccggtggggattcatgaccagagtagacttgacttattacgccattatcgtcttcttcctaaaagaaaagatgaggaagctcaaatatgcagcacttgtgctgctcttgctttctatctgcaacacttgtttttcagagatcatatttgaggagcgatttgatgatgggtggcagagccggtgggtgaagtctgattggaaaagaagtgaaggaaaggctggttcattcaagcacacagcgggttcatggtctgctgatccagatgataaaggtcttcagacgactactgatgccagacattttgccatctctgcaaagatcccagaattcaacaacaaaaacagaacactagtattacagtactcgatcaaatttgagcaggatatcgagtgtggtggtggttatatcaagcttatgtctggatatgtcaaccaaaaaaaagtttggaggcgacactccttacagtgtgatgttcggaccagatttatgtgggacacaaacgaagaagcttcatttgatcctttcataccaagggcaaaattatcctatcaagaaggatttgcagtgtgaaactgacaaattaacccatttctacacgttcgtactcaggcctgatgcaagctatagtgtcttaatagatggcagggaaagagattctgggagcatgtatacggattgggatatccttccgccacgaaaaatcaaagatgtaaatgctaaaaagcctgcagattgggaggaaagagaatatattgatgacccggatcaagtcaagccagagggctacgattcaattccaagagaaattcccgatcccaaagcaaaacagcctgatacctgggatgaagatgaggatggagtatggagaccaccaaagataccaaatccagcatacaaagggccatggaaaccaaagaaaattaaaaatccaaattacaaggggaaatggaagattccgtggattgataacccagaatttgaagatgatcctgacctatatgtgctcaagcctattaaatatgtgggaatcgaggtttggcaggtaaaagctggatctgtttttgacaacgttttgatctgtgatgatccggagtatgcaaaagaagtggtgcaagaagttttcacccatagagagatggaaaaggaagccttggaggaagcagagaaaataaagaaagcacgtgaggaagaggaagcaaaaagggcaagggaggaaggtgaaagaaggaagagagaaagaggttatgatagaaggcacagagataaggactattacagggacagatacagaaagcgccattatgacgattatcatgatgagctgtaagatgttgtagcggttttggatatagtctcattgtctgtatcattttcatctttgcatttacatgtaacacgcggattgtagcatctcgattgttgtcttaacgcacgcagataaattgcatattgtgcgagttttcaactcacttttttagctttataattaccaaaatttaatgcaaggagcaagttacatttaaggtgttttgttttgtgtcttgtgaagttggcgtgttcagttcataatctatttcatgcatttctaactaaaagttttagccatatttggttttggatattaaccaaattcaatcttaccgaattgaaaaacgacaactaacaaaaccaaccgaaccaattgattatttt

SEQ ID NO: 22 - DNA, 1548 bp, Artificial Sequence
  Synthetic Construct
gagtatctccagtatgttgacaatagcgcatttgctgttgatggtgttctttctgataaccccataactccatctgctgcatttgattgcttatcaaacttggctacgaacaaaactcaagcaaaaccgctgattatatcaaatgaaggagcaagtggtgattacccgggttgtacagatatggcatataaaaaagctgtttccgatggtgcagatgtcattgattgtcctgttcaaataacgagtgatggcgtacctatttgtttaggctccataaaccttcttgagagaacaaatgtcgctaagtcgttgaaatttataaattatacaacacccattcccgaccttcattctgatgctggaatatttacatttatgctcacttgggaccagattcggactctaagacctttaatgtacaacccatatcaagaatacggtttatcaagaaatccacgatacgcaaatgatggaaactttatgagactatctgactttttggagtttgctagtaatgctgcttcaatttctggtgtcttaatcaacattaagaatgcagcttacttagctgttaatcgaagtttaagtgtgacagatgcggtgatggacgtgttgaataattcaagtataaataacaagagaatcttgattgagtcaccggatagtgctgttttgaaactgctcaaggcaaaaagcaacaaccatgagcttgtgtatgaagtggatgagattataagcgatgcactgaattcaacaattgaggatatcagtgaatttgctaatacagttattattcgcaagggatctctgtttcctagttccgacggatttattgttgagcagactgatgttgttgcaaagcttcacgctttcaagctccaggtgttcgtacaagtatttaataatgagttcatttcacaaccatgggacttcttttcggacccgtatgtcgaaattaacacgcatgttaatggagctgaagtcgatggtgttattgcatcttatccagctactccttccaaatatagaaggaaccgttgtctaagtctaccagcaaatgagagaccaccttacatgcttccagcgtcacccggtgagttgttctcgctaatgacacccggatcgatgccaccagctgaggctccttaccctgttttaaacgattctgacgtggttcaaggacccatacctcctttggcaaaacagtctccgccatctagcaatgggaccgcagcagcaccacaaccttcaccaagtggccaaccaccgaaaaccatggtccgaattttgttttctgtgatggctattgttgggacaaatgtattattgtattgaatggacaactacgcagtctttttttattatgtgtaatgagttgatggatattaattaaggactactttgtcactaggagtattgtgtatgactttgttttaatttgatatttgtaagtgtcatattatctgtggaatatatatacgtgtaaaaggacacctgctagcgaaagatctattttcttcagaaaacctttgattattatatactatc

SEQ ID NO: 23 - DNA, 543 bp, Artificial Sequence
  Synthetic Construct
tacttttgttttcgtgataacctaggtagcgtttggtatgttagaacggaatgcggaatgaaatggatcactacgagggaatgaaaaataaagcgtttggttaatcaccggaaaggaatcgcccattctacaaggtattctattcctttaaaatcatttcatccaccccttgtttttttcattctattccctcttacaattttcattaacacaacctaccacctaccaccactacctaccaccactgtcaccgcccaccaccgcagccatccatcaccgtcacccatcactaccgctgctaaccaccatcgcttgttccacaaaccaccacctccagctccaccacatatcaccacccaccatcaccgccactgaccaccacccaccaccgccactaaccatcgattttattcattcgttttttcttgtctatccaacaacacaaagtaatgttttgtttcattcccacatggcaaccaaacaagacatggaatggtaatgatccattccattaccttgtccattccattacctcgtccattc

SEQ ID NO: 24 - DNA, 2753 bp, Artificial Sequence
  Synthetic Construct
aatatgttagttgatatatggacccaagaaacattaactaagaaccaagaatcaatgaatgcaaaatgactaatttgtggggttttcagtttctcatattttaatgtacaaagaaaaagagatttatttatgtatatatagaggtactgatcactcttcttactcaaattaagctacttataactcttgtaaaacaagagagtcaagcttttttctaaataataatatgggcaattacaagtttatcgaaactatctttgttcctatatttattttacttctatcattgaactatgttatgccatgcaacttaacaaccaatttttacgataaaacatgccctaatgcacttgcaaccattagacgttctgttctggcctcggtgtctagtaatcctcggaatgcagccttgctcgttcgccttcattttcacgattgcttcgttcggggttgtgatgcatctcttttgttaaatggtactggtagtgagcagctagcagcagcgaataacggtgtgttgggctatgaacttatagatgctgccaaagctgcggttgagtgcgtatgtcctggagttgtttcgtgtgctgatatactcgcagttgctgctcgtgatgcttctgttgcggttggaggaccatcatggacagtaagacttggaagaagagattctacaggttcaaacgcgcaacaagccggaactgatcttccaagagggaactttaatttaggtcaacttattagtaactttgctagtaagggactcaatgaacgagaaatggtcgccttatcagggtctcacacacttgggcaggccagatgtgttaggtttaggggcaggatatacggcagctccttaagaatagatgctgcttttaatacctcccttagcgcagtctgcccaccgactgcacctaatggcgatggtaacttgcgaccacttgacttacaaacaccaaatagattcgacaacaactacttcaggaatctagtcagtagtaggggtcttcttatatcagaccaagtactctttaatggagactcagctgatagcattgtgactgagtacgttgacaatcctgcaacgtttgcttctgattttgctgctgccatggttaagatgagtgaaattgatgtgcttactggtgctagtgggattataaggacactttgctctagggcaagttaataaaaaaaatatatatgtgagatgataagatgtcaccgtcgcatgttatgcagtcacaaacaagtattctgcatactttgttgatcatataattaataaaagtcttctctcaaccaagctttctactttaatattccattgatgaaaactataatttactcttttagttattttgatgatctaacctaatttcacatttactttctattttagtttaagatactaacaactagaggactcatgtcatataatcatcatcagctaatatacttgtaacaatgttatatatttctatctcattgggaaaataatttatcaatattgatagaactcactatccattattaagagcatctccaatgcgggacaactttgaaatcagacccgccacctcagcatgccatattatttgaaatcagatccgccacctcactatgcatcattaaatcctcctatgctaaatcaatccaatcctccatctcgtcccacactagaatgggtctgccacggcagacctgacctcttttcactatctccttcaacgctagagaagtccaatttgattttagaccttgtttgtttctccgggttgaaaatgaacatcaaaagcgcctcaaactgtcgctactactcatcatacaccttgtcatatatttcaaacatgtaacaattcttgagaaattgttttattgaaggtaataaataaatcaatttatacattgaacaattcgatgcaattatagattagatgttcgacccaataggcaataggtaaactatgttttcctaatccaatgttgagctctaactttggaaaacaattatgatgtatattcaaactcactgaagcctttgatattgtgaacttctaatataataaatttatcattctccttataaagaaaataaagtaaatcatcatgctaactattttcaagagcttgttatccaagaaacaacaaaatataacaataacaataacaagaaaaagaaaaacacaagacaagtaagaacacaaatacaaatataacctggttaactcgatcacgttatcaggcaacatccataacaaaactacagagactttattatttcataacgcactagatatcgatttatagtagtcagattgcggttgtttacttgtcatcaagtttacgcaaccaatcgacctaatattcaaacgagtggagcccttaaatagttaaatgtttatcagtcatcgacttttaattcgttatttaccattaggctagaaggtatggtcacgccccactccaccacgtaccaccacccttcacgtcatcgccacatcacttcattctccaccacgatcctctatcaccaacctatgggtgtggtcatgatggtcctctccactccccttgaattgataggacaaaacaatcaaatgcgtcaaatttggaaaagtggagcgtggtgccctggtgatcttcacgaagaccacacatggtttgtcgaatggggcgatgtagccatgatctcacggcccacattgcagcgtcacgccctccaacttccctccatactgcatagccttagc

SEQ ID NO: 25 - DNA, 494 bp, Artificial Sequence
  Synthetic Construct
ggatcaaatcaaaagtgtaaccagtaactcccggcaatgaagtcaaagttttgtatgcttcagctggaacatcagcaaagtaggttgcaacaagaacattaagaccagagcaagctgattctagttcagagtaagccttggtgaaagctgccaattggtaggcctcgagatccttcacaagggtagcctcatcaaattggatccatgtagcacctgcttccttcaactcagcaataacttccttgtagactggaaggattttgtcaagaagggatagaagatcaaaggttttctccacaccctttgcaggtttcgatagcaacaagtagctgacagggccaacaaagactggaacggtgtcaactccaagagccttggcttccttgtattcagtcacagccttgtgggaagcataggtgaatttcacatcagggcccaactctgggacgatgaaatggtagttggtgtcaaaccacttggtcatttccatagca

SEQ ID NO: 26 - DNA, 1584 bp, Artificial Sequence
  Synthetic Construct
gcggtcattgcctagatccgcccctggtttgtatcactaaatctctagtcatggtgacagagagattaaaccctaacgctatcgccttcatctctctgcttctcctcgtttcagtagcttctgctaaagtgttgttcgaagaacgttttgaggatggatgggaaagccgctgggttaaatctgactggaagaaagaagataacatggctggagaatggaactacacttctggtaaatggaatggagatgccaatgataaaggtatccagaccagtgaagactacaggttctatgccatatctgctgagtatcctgaattcagcaacaaggataaaacactagtgttccagttttctgtgaagcatgagcagaagcttgattgcggtggtggctacatgaagttgctcagtggtgatgtggatcaaacgaaattcggcggtgataccccttacagtatcatgtttggacctgatatttgtggttatgctactaagaaagttcatgcgattcttacatacaatggggaaaacaaattgatcaagaaagatgttccctgtgagactgatcagcttacacatgtttacactttcgttctccgccctgatgctacctacagcatcttaattgataatgaagagaagcagacaggaagcttgtacgccgactgggatcttctcccaccaaagcaaataaaggaccccgaggctaaaaagcctgaagactgggacgataaggagttcattcctgatcctgaagataagaaacccgagggttatgatgacatcccaaaggaaattcctgatccagatgccaaaaagccagaagattgggatgatgaggaagatggtgaatggactgtcccgactattccaaaccccgagtacaagggtccatggaaggcaaagaaaatcaagaacccgagctacaaggggaagtggaaggcacctatgattgacaacccagattttaaggatgaccctgatctgtatgtcttccccaagttgaagtatgttggcatagagctatggcaggtaaaatctggaacattgtttgacaacgtcttgatatgcgatgatcctgaatacgccaaacaagtcgcagaagaaacatggggcaaacaaaaagatgctgagaaggcagcatttgaagagctagaaaagaagagggaggaggaggaatccaaggatgacccagttgattccgatgcggaggataatgatgatgctgaggcagacgaagaggatgaggctgaagaaagcgacgttaaggatgagctatagagaagaagagggggtttggagtgtgagagcagttagaattttaagatgttatattttgttttttccatgacatgacgtctaatttgaaaccctttttaaatagaggatagatattgagaaatgtaatgcagcagattttctgcccctcattttaattgctttcattcgaaactattgaaaacaagtggggaatttgtgtgtttttgttaccatgggtaaaaccgaatttttacctgttttgttttggttgcgtttaggttggtttttggcaaattaaaatctaaaacttatccgttaataaaaa

SEQ ID NO: 27 - DNA, 268 bp, Artificial Sequence
  Synthetic Construct
cagtcttctgggtggcagtcatggcatggactgttgtcataagaccctcaacaatgccaaacctatcgttgatcaccttggccaaaggagcaaggcagttagttgtgcagctagcgtttgaaacaatgttaagatcaggtgtgtattcgtgctcattgacacccacaacaaacatgggagcattgccactgggagcagagataacaaccttcttggcaccacccttcaagtgagcagcagccttgtccttgtctgtgaagacaccagt

SEQ ID NO: 28 - DNA, 847 bp, Artificial Sequence
  Synthetic Construct
ttttttttttaaacaaaagaaaacttgtaatctttattcaaatgtagcttaacagggaaatacaccaccttttttagtagacttggataggtgccctacccaatttacattacaatatatagcatacatgatacaacatagtacagtacaataaaaatacaatacaacacatagggaatgcaataccaacaccattatgcaaaatataacatagttcgattattcgttttaatgtgggaatcattcgtttaaaggtgtttagcacatttaacaaatgtaacccaatataaaacaatagtaaggtggtgttttagtaagttatattacgacatatagaggagagactagcgtcaaagaaaatcaagcaatctagttgaggtttgtaattgcacacctttttgcaagttttcttacccgagcttttaataacctcaattatacattctgctttagtgcaagtagctggttcaggtatagtgtggcactcaaaaatacatcctacgccggaaaattcgtaagttgctttggtaaatttttttccgaactgtagaaaccacttattctgtttataagcctcttctatcatcggtagactcattatgcccttcggaaataggctcttaggaaatgtgtaataatattcttttaaatttttgatacctgtcggtagagttattgtatctttgggtatgccaaccttagaacctcgtaaccgatcttcagggttgcggtcatctgcataggaagagcagatcagaagaatgaaagcaaagagaaccaacactgctttcttgtcgaagatgttcataatttttttctgtccggcaggctcagatattgaagacgg

SEQ ID NO: 29 - DNA, 402 bp, Artificial Sequence
  Synthetic Construct
gagagagagagagagagagagaaatggggaaagcgttaggaccgacaggggagtttttccggcgaagggatgagtggaggaagcatccgatgctcaccaaccagttccgtcacgccacccctggcctcggaatagccctcgttgcctttggtgtctacgtcgttggtgaaatggcctacaacaagatctatgctccttccccatcatcttctcactctcactgacagacagatgctgctgtgaagaatgatgatgacgatgcagattgtttcttgtttactgtttatgctcgtctcttgaataaattgacaaggattttatggaattgtaacaaaacactacaattgttgtttgatgctgctgcataccagtgttatgtttgttgttgccttacgtaaagct

SEQ ID NO: 30 - DNA, 1897 bp, Artificial Sequence
  Synthetic Construct
gatgatccagaagcttttgctgagctgaaggtgaaagagataaagaacggaaggttggctatgttttccatgtttgggttcttcgtacaagccattgtgaccgggaagggtccattggagaaccttgctgaccatcttgcggatccagtcagcaacaacgcttgggcttatgccacaaactttgttccgggaaagtgaaattgaatcggaagtgtatatattgctgaattactgaaaatctataaaatgaattttgaatatgtgaataggtaacgttgatcactatttatatatttgaggttgaagcagctacgaaaatacatgatttcgggtgtacaccctgatgtgcatgtagacttagaaaaagtcaatactacaaagcacgaaatacattttgtgctactcattaatgatgtagtaatcagaagatttcaagtgataaatttgccacaaagaacaagttgttaaggtatgtaattgttataattttctgtgtgtcttacaaagatgatacaaggaggtatttatacacaagggattatacaaactaaggaaactattgattgggcataataaatgagatcgacttgacttttgactaattgatgttgacttgggttgattggagtgatgtccgctaataaatacatttatttaagtacaaaattagtgaaaaatcattttcactatcatttttcaaacaaattataataccaaaacaaattaaaaatgcaacccctcacaacaagattcttgcttcaagaattttatttgacaaatagttttcatcttccaaatatatgaagtaatcagttagttcttcggcaattggtcctcacttcaccacttgatcccgttaatgggttaaggttactcatcttgaccattgcatttgcaaagtctgttgagaaagttgcagcatttgaactgtaagttctcacttgggcgtcagtggaaccaccattaaatagttcttgatcagaatgcaataagccattctggttcaacaaattactgaaatatgcattgtcaaatgacgttggagaaacaacgtctaatggagatagattattgtcaccaccacttgatggacaattcgcttgtaatgatgtagcgaatgatgaatttatgttgttttcgttgtagatgcgatttcgaaatacagtacaccttgcttggccaatagtatgagaccctgataaagcaaccatttcattagttgtaaatccttggcttgaaaaggatgaaatgagaccactaaggttcaaagtaggggcaggaatgttggcattagcggcgtttaaactagccgtggttgagtctctcctcccgaacaccaggttccaagtcggcccaccgagagctacaacggaatctcgagcagcagctgctaacacatcagcacacgagacaactcctgggcatgaactctctagttgagttttgatggtgtcaataacatcaaatcctctcagtgaattgttattaggtcgagctgttttttctcctgtgaaatttgcagtgtcgtccaataacacagatgcatcacatccattaacaaagcaatcatggaaatgaaggcgaagcaaagaggctcccatacgagcttcacttgacacagccgtattcacagcacttgatataatagttcggaaatttgggcacgttgtagtatagaagttggcagatagctgaccgtaactaggtccaaacagaaccaacaaacataatgcaaaagttgaaagtttaaaaaaagatgaaaagtttgataaagccatgcgtaaacataaacaagttataaataattgagaaagaagtggttagttgtgattattgatggtttgtgcagcatctatttatagtgatgttttttctgtaaggggaaggggtggtttat

SEQ ID NO: 31 - DNA, 2941 bp, Artificial Sequence
  Synthetic Construct
cgacgatcattatgttgttttttaaatgtagttaactttgtataatattaaagattaaaaacctaaaccatacaacttaggggtgttgggtagacagcaaatcattccagccggtttgtgagttgaccaaaccggctggttttagcagttgtgtaacaactggctagaccgaaaagttgcgaacccatcaaaagcgaaccaaaaactaaacaagggtctcaaaccaaagaacttgttttagattatatggttcgataattagacatttaaacctactccacttgtgtttattgcaattttatagtgtttgcaaaccaacaaaatatcatctttagaattggattcattgggggtatgcaaaagccttaccagaaaaggtacataaacaaccaaaagaaagcccaaaaagaagaaaacagaactcaaaacctaagacatgccatcaccaaccacagttattccatcactgtaacataaaatattttccatgtcaccatcgtgtgtcctccgtcgggtcaaatctcctcacttggcaagagcagcacggagcttcttggcggctgcaaccatgttcaggagtgcgggcttaacctctgtgtacttacgggtcttgagcccgcagtcagggttcacccacaagatattggtttccaaaacagcaagcattttgttgattctgtcgtggatttcctctgtcgatggaattcttggggagtggatgtcgtagacacctggaccgattccagcaccgtacttgactccctcacggaacacggagagaagcttctcatcagacctggagttctcgatggtgatgacatcagcatccatgttgatgatggaatggatgatgtcgttgaagtttgagtagcacatgtgtgtgtggatctgggtggtgtcttggaccccgacgttggtgattctgaaagagtggactgcccagtctaagtagaaagcatgctcggctttccttagtggcaatccttctcttaaagcagcctcgtcgatttggatgactgtgattcccgccttctcaagatcctcaacttcatccttaattgccaaggcaatctggtagcaagtttcaaatctgggctggtcgttacggacaaaggaccagttgagaatggtgactggaccagtgagcatacccttcatggggcgtttggtcatttcttgagccatggtggaccagaagacggtcatggccttggggcggctgacatcaccatagatgattgggggtttaacacagcgggacccataagattgcacccatccattagcagtgaaggcaaaaccagacaattgctcaccaaagtactcaaccatgtcgtttctctcgggttctccatgaacaagcacatcaatgtcaagttcttcttgaagttggacaactttgaagatttcctctttaatggctttgacatattcttcctcagagatctttttagccttgtactcacggcgaactcttctgagctcaatggtttgagggaaagaaccgatggtggtggtgggaagaattggaaggttaagtttcttctgttgggcatccaatctggcactcacgttggtggcacggcggtggtcagaacccctcaaagctgctgcagccttctggacagcttcattggtgactcttggggagcacttccttgaggcctgagcagcggcattagctgcaaagaatgcctcatccttctgaccagcaagagctttggccaaagcaccaacttcaatcaccttttgagcagcaaatgcgagccaagacttgatctcatcgtccaacttggtttcgttcacaagatccacagcagtgtgttggagtgatgatgaggtggacacaacaagtttatctttgccgacaatagcctcaagagactcaaggacactaagagatcctgcaagatcgtttgcccagatgtttctgccatcaacaacaccagcaaagaggtatttgcctgatgggaagctggtcttgattaaatcaagagtcttttcaccacggatcaaatcaaaagtgtaaccagccactccgggcaaggtagtaagggttttgaatgcatcagcaggaatatcagcaaagtaggtggcaacaaggacattaagcccagagcaagctgattcaagatctgtgtaagccttggtgaaagcttgcaattggtgggcctcaagatccttcacaagggtagcttcatcgaactggatccatgttgcaccagctgccttcaactcagcaatcacttccttgtagattggaaggattttgtcaagaagggaaagaagatcgaaagttttttcaacaccctttgcaggcttagatagcaacaagtagctgactggaccaacaaaaacagggacggtatcaactccaagagctttagcctctttgtattcattcacagccttgtgggaagcatacgagaatttcacttcagggcccaactcggggacaatgtaatggtagttggtgtcgaaccacttggtcatttccatagcagggacagaggcatttcctctagccattgagaagtaagtgtcgaaaccgatctcaccaccgttccagttgtacctcggtgggacggcaccaatcatggtggtggtgtcaagaacctgatcatagtaagagaaagtgttgctgggaatatacttgatgccagcatcagccatttgtttccagatggatgatctgagatcagcagcaactgtctgcagatcctcagcactgctcttgccatcccagaaagactcaagagcgaacttcagctctctcttgggacccatacggggataaccgacaatgtgagacgccattgatctttaaaaaggtgagaaaaagggaaggtccgaataaagggattggttggatcttcctgatagatagagagagagagatagaggagaagatggtggagaccaaaatatatgggtgtgattt

SEQ ID NO: 32 - DNA, 6565 bp, Artificial Sequence
  Synthetic Construct
tccatcgattattttcatcgatgaaattgattcgattgctcccaaaagagataaaacaaacggagaagttgaaagaagaattgtctcgcagcttttgactctaatggacgggctgaaatcacgggcgcatgttattgtcatgggtgcaaccaaccgtcccaacagcattgaccctgccctgaggagatttggcaggtttgaccgggaaatcgatatcggtgttcctgatgaagtgggccggcttgaggtgctccgcattcataccaagaacatgaagctggctgaggatgtggacttggaaaagatttctaaagagacacatggatatgtcggggctgatctggctgctttgtgtactgagtcagcactccagtgtattcgtgagaagatggatgtgatcgatttggaagatgagtcgattgatgctgagattcttaactccatggcggttacaaatgagcacttccaaactgcccttggtacaagtaacccgtctgctcttcgtgaaactgttgttgaagttcctaatgttagttgggaagatattggtgggcttgaaaatgttaaaagagagctccaagagactgtacaatatccagtcgaacaccctgaaaagttcgagaagtttggcatgtcaccatctaaaggagttctgttttacgggccacctgggtgcggtaaaactcttctagccaaggctatagcgaatgaatgccaagctaacttcatcagcatcaagggtccagaattgctaaccatgtggtttggtgaaagtgaagctaatgttcgtgagatcttcgataaggcccgtggctccgccccttgtgtcttgttttttgatgagctggactccattgctacccagagagggagcagccaaggagatgcaggaggtgcagctgatagggttctcaatcagcttttgactgaaatggacgggatgtcggctaaaaagactgttttcattatcggtgcaactaaccgtcctgacatcattgatccagctcttttgaggcctggtcgtcttgatcagttgatttacattcctctccctgatgaagagtcacgttattcgattttcaaatcagctttaaggaagtcacctgttgctaaggacgttgatcttcacgctcttgctaagtacactcagggattcagcggtgctgacatcactgaaatctgccagcgtgcgtgtaaatacgccatccgtgagaacattgagaaggatattgagagggagaagagaaggagcgagaacccggaggctatggaggaggatgtggaagatgatgtgccggagatcaaggcggcgcattttgaagagtcgatgaagtttgcgcgaagaagtgttagtgatgctgacatacgcaagtaccaggcgtttgctcagacactgcagcagtctagagggtttgggtctgagtttaggttctctgagactagcggtgcagccgctagtgctggtgggtctgacccgtttgctgcttcagctggtgcggcggatgatgacgatttatacaactagcgttaaaggcacactcgtcgtatcttttacttttgtaatttatgtaaaacttgatgcttttgctttacaagacttgatcccctttgcgttggtggaaaacgtttccaaaacgtttcttgaatttatggattaacttcgtaaatttgtctttaccttctgttttatagttttaccataagtattaagttggttatgatggataggcaattagctgtaggaatactatactgatctgaagttcatatgctttgatttgactgtttcatggtcatgtttgtggtaaaacgtcgtcaatctgacgttttcacacgcaaggcagcaagtgtcggcaacctgttcatctccgctctccaaaccataacttgacatttaaagggcacccaagagacccatttaaacataaaattagtgccaaattcagcctcaagctgcatcaccttcctgaccgattgggttgaaaacagctttcctttgtctgcagcccaagaccaacgatcttcagtacctcttatctctaacctgctaatgcagcccactagctgcagccactgatcaaaaacctccacaaaattaatcctcgacgtccagtttgcaaccagcacccaattattaccaacccgcttcttccgatccaaaactgaacatcccttcatactttccagactatacaataatggccatcgatatttaagagctacatctccaagccagacatcaaaccagaaacgagctgaggtattcatacccactttaacccttatgaaagtatgtaacgaaacattatcaatcttcagtgagcttccagatttcacaattgctttccatgttccagcagcattattacaaaccggcaacatctcccacatcttatttttgttatgacaagagtctattacacgacgccataagcagcccgggttgaccctatacctccaaactcatttaaacaagagcgcatcattcacatctttcaatctcttaatttccaaaccaccacaatttttcggtctagtcacctgatcccaactcacccaatggatcttgttattgtctaaggaacccgaccaaaggaatttcttcataattctttcaaggctatcaatgacttttcggggagctttatacaaagaaaaatagtatatcagaagactttcaaggaccgatttaataagaataattcgaccaccaatcgacaagagcttcgctttccaaagagccaatctagcttcaaacacctcaacaactttcttccaattaccacatctattcatatcatgttcctacacttcaattattattattaccaagtatttatataatcaagcatcatactatatgtgtgttctcattaatagttcatataatatttatatacatataggtccctttaatttagtcgagttaagttcggcccggtttagtaagctcgtcaatgtcccacacgtgctcgtcctcataatagcactaagtaccaatatgattctagttctcgtttcataccttccaatctttcagctgactatgttgactgttcggttgttcatcctttgactgatgttgaccaaatacaccaatgaacctatcatcatcacaatttcccttcgctaccaatatacattctccactgtttcatcttatgtccctcttctgtagactactagtagccatctcaattaatattactcatcatcttaaataactaaattagtgaataagctaggtggggttgtagtacttaccttggttgctacttctaatcgattccataatagttcctgaatagtatcatcatcgccacttaggcgactaacggagctgccatcaaatcccgaacctcagattaaaccgatgtcgccgtcactcttactcggaaccttgctcgaaacccagatcaaaacagtaccctaatcaaaccctaaacccgattgagaccatcaaccagtgccacaaccgtcgtcttctgctctgttccgccaccgtgcagaccggccagccgaaggctggggtcggtgaccctctctctctctctaactttctctctctaaacaattcctccaatggcgacggcgggcgacacctaccagagcggcggagccggcggcaagcttaggaaacggccaattcgccgtagctccaaaacgacgccgtacgatcgacctcctattgccctcagaaacaataataataataataacaagggcggcgaccctcactggcagccctttgttgctgagaacgccgcttttaatcactactataaggagcaagggatcgtacgtccggatgagtgggatgctttcatggaatgtttaaagaagccgcttcctgctgcttttaggatcaattccagtagccaattctgtatggatctcaaatcgcagttacaggatgactttaccaaatctcttcaggcagaggctacagatgggaacgaccctgagggcatcaaggcattgccttggtatcctgataactttgcttggcagtcaagtttttcaagaatgcaactgagaaagaacaagagtcttgagaggtttcatgagttcctaaagcaagaaaatgaaattggaaacattacaagacaggaggctgttagcatggtaccaccactgttcttggatgtacatcgagatcattttgttattgacttgtgtgcagccccaggttcaaaaacgtttcaacttcttgaaatgatacaccgggaatcggatgttggatccttaccaagtggaatggttgtagcaaatgatgttgatgtacaacgatgtaatcttcttattcatcaaacaaagagaatgtgcactgccaacttggttgtgactaatcatgaagcccaacattttccaagttgtcacttgaaaaagaatcatgcgaatgcttctgagttgggtatgactcagctgttatttgaccgcgtcctgtgtgatgttccatgcagtggagacggcactctccgcaaagctcctgatatctggagaaaatggagtgtggggatggctaatggtgttcattgtctgcaacttcagatagccatgcgaggtttggcattgcttagaattgatggaagaatggtttactcaacatgctctatgaatccagtggaaaatgaggctgtagtttctgagattctgaggaggtgtggagaatctgttgagcttgttgacgtatcttgtgagctccctcagcttattcgcaggccaggtctcaaaagttggaaggttcgagacaaaggtgtgtggttagcttctcataaagatgcttgcaaacatggaagaagtgggattgttcccagcatgtttccttctgggcaagctcatttagagacatctgctgatagcaatgagaaatcaacaacaataactggtgctgaaataattgaagatgggaattctaacaacggggatgttgatgctgctgctgatgttttagtgcctgaagtttcatcacttccgttagagcgttgcatgcgttttgtgcctcatgaccaaaacaccggagccttttttgttgctgttttccacaaagtatctcaattaccagtcttacaaacaaagtcgaacaaacattcgcgggaacgagaatctgatagtgatgaaaaagtgaaacaagaagataaagatgttaatggaatggatgtgagtactgaagaagcaaccactgaggcaggtgtactcgatgaagaatctgatgaaaaggtgaaacaggcagaggataaaggtgttaatggaattgatgcaagtaatgaagaggcaatagcagttgctggtgtacccgtcaaagaatctgatgaagagttgttggactctaatccaagtaaaaagcttagagttcagagtccagataaaccaaaatctcgtgcggagaaggccaatgataaaagaaagttgcagattcaaggcaagtggtttggagttgaccccgttgttttcttcaaagatgatgctatactcactggtatcaaggatttttatggcatcaaagaatcttttccgtttgaccatcaccttattacacgaaatagtgataccaatcatgtgaagagaatttactatatatcagaatctgtcaagaatgttgtggaactcaacttcctagctggcgagcaactcaagataacttcaatcggtctgaagatgtttgaaaggcaaacatcaagagaaggggttgccacagcttgtgtgttcagaatatcttcagaaggtttgccattgcttcttccacatatgactaaacagatagcttgtgcatctcctggggatttcaagcatttattgcaatacaaatccatcaaattcccagatttagttgatgatggattccgtgaaaaggtttcgagtctttctttgggttgttgtgtggtggttctcaaccaaggtaatgagggctcatctgatccgcctgtagtggataaatcaacgattgccattggttgctggaaagggcgttctagtttatcagtgatggtgacagctatcgactgtcaggaaatgctggcaaggttattggttcggtttgataccaaaaacggatcttcaacagacattaattgtgaggaggcagcaaacgttgatgatgatgcgcaagtgaaggaagtttctgaaaccgtaacgtcatcggttgatgttgaagtaaacaaagaatagttatggttgttttattgtcaactaccggattttgtactctggttgaaagggaattatgatacatgagaaagtaggacatggaatttggagagggctttctgattgttgtacctgaaagacaatacttttgattgagacttgttctatttatttaggtagcttttcatgtgctataagccctctttaccatttgcttctttcaaaggttggtttgtgtccagatttccttaaaccaatagtcatgatttatgaaggtgttccattcttgaagctggtcataccttttgattgccgttgaacaacttccccaataacaccaattatccaatggcggactacttatatcttaaagttgtttttcatgtttttagtgatctacaagatatgttgcaaccga

SEQ ID NO: 33 - DNA, 897 bp, Artificial Sequence
  Synthetic Construct
ataataataataataatcacaataataataatcacatctaaacaggttaggttcgagtttaggattaaaatcacatctaaacatatcatagtgtttatcgtcgttgtatgtggcttaataaaccacaatatgcattcaacacattagaacaagggtatgtatctcaagactacctgtttcacattagaaatcagtttgaaatgaaaaccacacgtatatatcagtttcatgattagaaacccagacgacaatcaaacaagataaaaaccagaatattcatttattcaacaatcacaaagagaacccatagaaaccatatccaacaactgttacagcaacttcaaacaacataacttttaaaccgccaaattcaggcatcaacagacaaccaccacaaacatctaaaacccaccacgcagacgaaggacaaggtgcagggtcgactccttctggatgttataatccgccagagtcctaccatcttccagctgcttccctgcaaagatcaacctctgctggtctggtgggatcccctccttgtcctggatcttggccttgacgttatcgatagtatcagagctttcaacttccaaagtgatggtcttcccggtcagagtcttaacgaaaatctgcataccacctctcagacggagcaccaaatggagagtggactccttctggatgttgtagtcggccaaggtacgaccatcttcaagctgttttccagcaaagatcaatctctgttggtctggtgggataccctctttgtcttggatcttggccttgacattgtctatggtgtctgagctttcaacttccaaagtaatcgtctttccggtgagtgtcttgacaaagatctgcatcccacctcttagcctgagcacaaggtgaagagtt

SEQ ID NO: 34 - DNA, 959 bp, Artificial Sequence
  Synthetic Construct
tttacacgtcgttcatcaaagttttaatgatacaaattcgagttactttgagttttgccgccgcacaacgcacggctggtcaaaacactagttggttttaacacatgtaaaacaaatataacatttattgtactttaactagttatttgacgaaaaaacaaaacacgtcttcttatacaagacgggtcaaaactcaaaacacatacaaaacgatcttaaaaactaaaggttctagttaagaaaaagatgtatgcccgatacataacattatcatctctgagagcaagaagatcggtatcatcaaaattaaaacataaagataattatagaggaagttcaccgctatcagcaattgtaactttgctttttggtgatccactttgtgtcccctcggcttccatcttgtagacgacatccatcccggacaacactttcccaaacacaacatgtctaccatccaaccagctggttgtgaccgttgtgatgaagaattgtgacccattcgtgtttttttccagcatttgccattgacaatagtcctggtccggtatgcttgatcttgaaattttcatcattgaatttttctccatagattgattctcctcctcttccatctccaagtgtgaaatcaccaccttgaatcatgaagctggggataatcctgtggaacgcgcttcccttgtagtgaagaggtttcccactcttcccggttcctttctcccctgtgcacaatgatctgaaattttcagctgttttaggaactgtattcccaaagagaccgaaaacaattcttccagcaggttttccagcaatctcgacatcaaaatatactttgtgagtcacctcggtcaaatctttgtttgatttcttagcctgagtgagtgctagggttccgatgaggagtagagaggagaggagtagagctgacggcaacttcattttcgttaacgatggagtcc

SEQ ID NO: 35 - DNA, 2529 bp, Artificial Sequence
  Synthetic Construct
agactaggattagaaaagaaactcatatttcgctaaaccaatttggcttcatgccagggcggtcaactattgaagcaatccacattctaaggagactaatggagaagtatagggagaaaagacgggacttgcatatggtgtttattgacctagaaaaggcatacgacagtgtcccacgtaagctactttgggatactttggaaggtaaaggggtgccggggaagtatatagatataattaaggatatgtatgaaaggtctgtcaccagcgttcgggctcctgtaggggatactgattcctttcctgtggagattggactccatcaagggtctgctttgagcccattcctctttgcgatggtactggatgagctgtccaaatcgattcaggaggcagttccgtggtgtatgctttttgctgatgatattgtgttagtggcggagaccaaagaaagtctgaatgcgagggtagaagagtggagagcatcgttagagagcaaggggttaaggattagtcgctcgaagacagagtacctctactgtggtttcagtggtgcagatgacgttagtgacacgcagatcaccatcgatggtcaagcggtcccgaagacaacgactttcaaatatctaggatcttttgttcaaaatgatggagagatagatatagatgtaacccaccctatccagccggctggcgtagatggaaagcagcctctggggttttgtgcgacaggaggttcccaactaaactaaagggaaaattttatagggttgcagtccgacctgctatgatttatggggcaaaacaatcataataccaaaatagaatacttaatgagaggctaaaacacctcttaattcaacataacacaaagactcaagacaccaacaataacaataaactcaaacagcaagacaaccaaccagaccagccataaccatggctttatttaaaatcttaaatgaaataaaaacaacccttcttcaacacataaaacatactactaatactccttttcttcattctctttccagtccttgccacacttcaacacagaccttggtgacccacagcagcttccttccttcttcacaaacttttggtacacaattgaaccttcacatccaagctcttcataactcttctcttccacactgtacccctcgatttcgatcgcgcaagtcgcctccaaacgctttgacgccacttctgcacctatggcaatagaaaactcgcccggttcaaaacaagccagtacccgttgcactagctgttccaggcttacctcattaaaatcataaccgactgcctcaaagctagcgtaactgaacccgtcttcaggtgtaacatggatagtcgaaattgcattgtcttcaatagagttcatagagtatccacacgggtcaaactcgaaatcacaaatatcggattttggaaggattgaacgaatgccagattcgttagtcatggttgcagccgagctcgattctgttttgtagaaaactgatgctttctctctgtccaaactagtcatgcacatctcgagtgtgtacacttgatcacgaactgaatcgggtcctgcagaagcagaataaacaaaccatttttggggcttatcgaacccacccatcacgtatgccttgctggataaaccgagcttttcaaaatgagcatcaagcacagaaacttcttcggtgaagctgcggtgtgggtaagattgagcattggggaagataaaactgccacgagtgtacctcactgaccggacagtgagggaaattgattctgcaagcttcaaaataggcgggattgaaagaagcagcttggtcgtcccacaagttttaataaccatcttatacggaaaaataaacagactagactcagacagaacatacgagtccaaatattcattcgacagcgaagaaacaattgtgcattctgcagggttcaaaatctcatcgatttgggcttttgacaatgcccggagaccctgaccttgcgggtcaacaaagatgctaggctcaaagaatgtaatttcaagccgtttttcgtaaccttcaaaaccaattgcagacattttgacagaagcaaacaagaaagagactgagaaccgaagaaaaggaggaaaaagggggaactaattataactaataaactaatttgaaagcagtaaataaatcacgccaaacgacgtgaacgggacactaatttgagggatatcaggatggtttgcgagcgcagttggagtaagcagccgatctgaatttcttgattcctccgtttggtcgaacgtcttcaatgctgtatcctaggggagcttcataaaacgatctactactactactactagactttttcccacctttagtctccattagatcattcaatttttcagtatgaatttcgtatgtatggctatataacgacggttaaacgacgaatcaactatccgatgttttatgaatgttaatagccgacgatgttgaacgataatgaagaagaaattagggttatcgtgaacgcaatgtt

SEQ ID NO: 36 - DNA, 2078 bp, Artificial Sequence
  Synthetic Construct
gataacgatggttttgatataattctgcataaacataaaaacgaaaatataacaatttatgtcagtcttacaataacaggtcttctcataagtagggttgttcgtttttatctaaaaccccggaccgaatccgaagcacccgaacccaagttagagaatacatgaaaaacccaaacccgaataacccaaacatgaccaacctgaaccttaaatggtttggttatgggtcactttttcccagtcaaaaatccgaaaaacccgaaatctgaaacccgaacatttaatggctttttttttttaaaagaatatatcatctaagtatagagtctttggtatcttaaacattaagtttcaggacttttgaacaccattatatcatattgtaaaaaaaaattaaaaaaaaaactgaattttgataatatttttaaataacatgatgaattttgttgatattttgcgaaagaaaaaaaatatattacatttaaacccgaaaactaaacaacccggctcgaactaacctgaaccagaataacttcaacccgaactttaaatggttcggttatcgggtcactttttctcaacaaaaaaacccgaaaacccgaaaaaataacccgaagaacacccctacttaaagggacatctttaccacttaaacactgctcttaaaaaaaggtaaataaaatgaacaactacattgattaaaaatgtaagatattacatgcttgaaaactactactattaaaactacctaaggcgaacttgttcttcaaagcctttaacatttcctctaggattaccttcaaaccttctgagcttaaaatgcttgagttgttcatcctgcagggctgcagccgccttcccgcctagcgccaaatccaccagtatccgcccaaccactggagccatcttgaacccatgacccgaaaacccaccggctatcaccacatcttcaccaaactcaccacccaagaagtcaatcacgtaatcgccatctggtgtcaatgaatacaagcaagattggcgcatcactggcccgtcttcccaatcaactcgatcgccaaacgttgattttatccattctttcagatatccaatcaccattgctgtcctttcatcacctggtggcgctgcgccccatgtcctctgatccggatcgcatccccgaccaccatgaaccgcaactttaatcaaatccgggaactccaaagccggtgttccgtagatatacggatcaccgtagctcgcaaacgtcggaaaccctgctccgatggtgtaatcaacctcatatccctctttaatcttccagtaaaacaggctcgtttcgatcggttgtataggcaaatgcataccaccattacaaaccttccctaccagtttactcatccaagcacccgcggtcaccacacatttcttcccccaaaacttctcccccgatccacccgtaaccaaaactccgcctcgatgtttatcaaccgcgatatctacaatctccgtcttgtccttaagtatcgccccatacttgatcgcgagtgtttgaaacatcgcgactgctttcgtaggcttcaaaacacccccaagctcagtcactaccgcaatccaatcatctggaaaatcaacaactccagagaaccgtgttctcgcttcacgacgatcgagaacacggtaattcatcgagtgcgttctacaattagcaatgatcgagacaagagaggtattatcagctggacctaaatccaaatgcttggtttcgtagttgacattgtaaccaatttctgactccaattctctccataacctcttcgattcaatcgccatcggcgaataatattcttcaggatacgttgctcggattgttctagactctccgtgagacgatccacaatggtgtaggaagtcgaattgctcgagaagtagggttttctggccgcgtttagcggcctggtacgcggtcgagcttcccattacgcctgctccgatcacgattaaatcgaattgggatgaatgatccatggctttttaagtaagtaatttgaagttttgagtatgaattgaggatttgttttgttttaggggtgaaa

SEQ ID NO: 37 - DNA, 2130 bp, Artificial Sequence
  Synthetic Construct
gggagttttcacaaagtatgttgtgttatcgtgtgatctttgccaacataaatacagtcgatttaagaagaaagaacaaagattgagtgaacaaaagagcagtaacagatgtaataattaaaaagctttatttattgaactaaggacgaacttcacaccataaccactgaaaaaagatttcaacataaagcaaaagaacacagaaacaaaattacaagactcaaatacagtaacactttcaacaacataggatttctctaattgtttatctgtaggttcggtagttgggggtgtacatacagctctcagcatacttcactagatcagtagggcggggaagacgtgttgccaaaccaagatcatatgctttggcagcaacatttgcagcaatatgagctgaaatcttcctgatgttagtaagtggggggtaaatcatccctttatcgtagtgctcttgtgttacttgattagccaatgcttcggatgctgccaaaagcatctcatcatgtacacggatcgcaccagagatgaccaacccaaaaccaagtccagggaaaatgtaagcattgtttgcctggccaggaataaagaggttgccattgtattcataaggatcaaaaggactgccactggaaaatattgcacggccctcggtccaggtgtaagcttgttcagcagtgcactcagcttgtgaggtcggattggagagagccataataagtggtctctcgttgaaggctgccatggcctcaactacttcctttgtaaatgtctgtcctaccccagatgttccgatcaagacagatggtttgatggccttgacagcatctaagagagttgtaacaggctcatattcatgagcccatggtttcttaaagtgctgaagggattctttacgagaaccaacaatcaaccccttcgagtccacaagccatatcttcttacgagcctcttcaacgggaatatttgtctttgttgatatttcaagagctataagctctgctataccagtccctgcttccccagcaccaagaaacaagaatgtgtgatcagccaagctaccgccaagtaatttcagtgatgcaacaagaccagcaagaaccactgatgcagtcccctgtatatcatcattgaacaccaaatgactggttctatatttggacaacagctcaaatgcattgtggtttgcaaagtcttcaaactgcacgaggactttttcaccgtagttctgcttgactgcagacatgaactcttcaagcaagtcataatattcctttccggtggccctcttttgtttaagaccaatatagaactcatcatccagaagcttttgattgtttgtccccacatcaatggtaacaggcaagcacgcagaaggcctaactcctccaagtgcagtatataaagcaagtttcccaactggaattcccataccctgacaaccaagatctcccaatcccaagatacgctcaccatcggtcaccacaataacttgaatgttcctctcaggccagttcctgagcacctcaagaatctttcccttctctttcaaactaatgtagagaccctgaggacgtttgaatatactcccatatttctggcaagcctcaccaactgtgggagtgtacacaacagggagaagttcttccacattatcaataagaagcttgtaaaaaagcctctcgtttcgctcctcgagttccatcatagccacatatttgtgtaagggaacttcataacttcgaatattttgcatcaatttcttctcctgaagctcctgtgttgaaatagccggaggcaacaaaccacgcaagtaatgcgaatctctttccctctcagtgaacgcaagccccttgttatggtgtggatccctcagcaaagtgtatccactggcgacggagacagtccagggagtgataagctggtcctccgatgcacgatcttcaccgtacacgtcatcaacgccaccaccaacggcggacttagggtcaactactgactctccctgcttctccatttcttttcttttctttcttgtctgctgttgttgaaaatgttgaaatttgtaatgtaactgataattgaaatgaaattattatgtgatgaagtgtacggtgaagatcgtgcatc

SEQ ID NO: 38 - DNA, 1045 bp, Artificial Sequence
  Synthetic Construct
agttaagcccgaggattgggatgatgaggaggatggtgagtgggaagcacccaaaatcgaaaacccgaaatgtgaatctgcaccagggtgtggagaatggaggagaccgttgaagaggaacccagcttacaagggtaaatggcatgctccacttattgataatcccgcttacaagggtatatggaagcctcgcgagattccaaatcctgactactttgaacttgaaaagcctaactttgagccaattgctgcaattggtatcgagatctggactatgcaagatggaatcttgtttgacaacattttgatagcaagcgatgagaaaaccgcagcagaaatcagggacaccacatggaaaccgaagtttaaagtggaagaagagaaacagaagaccgaagaaaaatcagctgggcttgacgggctcaaaggaattcagaaagccgtgtttgatgtgctttataagatcgcagatctgcctttcttgggcgaccacaaggttaaagttttggaacttattgagaaggctgaaacacaaccaaacatcacaattggtgtcatagtttcaattgttgttgtgatcttcagcatcttgttgaagttgttgtttggtgggaagaaagcggcaccgaaagtgaatgtggcaccagaaaagaaggaagaaggggaggcgtcaaacacagccgaaggggaaggtgaaggtgtagttgaagaggaagagaaaaccgacgatgcggccgcggcgcctcctcgtagaaggaatagacgcgacacctgatgagagatggactgttgaagaaactatagttggtagtggtgtagttgatatttcaaggatgtttttgtttggttaaaacacagttttcttggtgtattaggaacaggaaaagctttttgtatgcgttgtaaatggatattattcatggattatgtataaccccttatattaatcattcatatctgagtataagcctactgaattaaagagttgataagattttgtaagtcgtaacaaaattgttcgataaatctttactcttctctcaat

SEQ ID NO: 39 - DNA, 1122 bp, Artificial Sequence
  Synthetic Construct
cctcagggacacaaagttcaacagggaagattgtggtcataccgcgagtagagttaaatagattctctcgtcagatttctgccttttattttacagatgactgcttgcgaggctgttgggagcttgatcctgcagcacttacaggactgcagctgcttgaacctaatcaatgttagtcaaactaattccaatagcaagttaacatggtgccatgtatacaagcaaccatcatagtatgtatttttcttaattacaaaagctaaatagaatcagatgataaacaagttgtacaatatagacttgtaaatttagcatcagagagcagaatgatactaccattcacttctaatgcgtatacattcaatgcaaaatactaccaatacataagaaaagcattccctttaggaaaacagatccaaagataaaaacttccactccacaagatacaagtcaacagaacaatgaagcaccaaattcgtcgaatgttgctgatatataattatatatactaaacattttttcatatacaatattatttctcctgtcattatgtcaaatcataactgataacataccgacagataaccggaaagattataagccaaaactttaggcaaataatttcaagattcatccatgaaaaccgcaccatgaccgttagctttcatgaacttcttcagcagaagcaccataatagcaatggtaagtccgacagccgcccatgtgaacaggcggtcatcagcaggtttctgaaccctcatcacctgctgctgatgtggttccactgtgtgtacagaagacgaagcagaaggtacagtgttagctacctgccttgataccattggggcttcaggttctgtgatttcttcagcggcaacttcatcattttgttcaggcggctgttcttcatgttcagtaatctctgtgctgatatcttgagggctctcagtctcaggagcttgttcacctgctgccgcatcgatggcggcagatggtggctgttctgaagcatcttcagagctctgttgagggactggaggtgccttgctgatcatatattcatgaatctcgtcaatgagcttttgacgttctggagtaccaaacttaggggcagcttcacga

SEQ ID NO: 40 - DNA, 636 bp, Artificial Sequence
  Synthetic Construct
cacaaataatgaaatgatcataattcaatcggggttataagggcaaatatacggtgcagattgacaaaaacagataaatcaattcaaataccctatcaagttaagcttcaagtatccacctttctgcaacccaaaacagcaaacgtaataaaattacaaataaagcgttaaaattcagcaaccaaattataaaaccctaaattccaattacaagcatattatcacaatcactgttacacaattcacaatttattcatacacgatcaaattcaagcgtttaaaaagttattatcatccatcaaacttaagaaaataccatcataaccacaatcatctcaattgtagtaaagttcaagacccatgccatcatgaatctcgtagtccttgagggtgatatgatccttgtagatgttgtaccacttctggatcctaatcttgtctgcgcgtgttcctgtctgtgctgcaacgagcttcttcaggtcgccgattgtgtcgtcgtcgttgcacttcactcggaccttcttccccaaccgatcgttcaacaccacctcgatcatcttcgttggtctttgattgtctcctgctgaataattgggtgtgtggagaatattaatttttttgaaaccctaattcctc

SEQ ID NO: 41 - DNA, 845 bp, Artificial Sequence
  Synthetic Construct
tttttttttttgattaagtaagtgttcatattcaaatctggaaggacaaaagtgcaattctcataaacacaaatactccataatcaaagtacaaacgatacatgaacaatattacacataagtccatcacaattggcaaattgtcattttaagtaaaaatatttaaagggataaacctgattcctcatcctcatcaaggcttccggtgtagacaaagtaaagtgtccaaatcaaaccaaacacaccaaaaaggatccacccgagaaggttgttgcttagcccaaacggaagcccggtcccttcggtgctcattcgctcgtccactagagccatggccgctgggctcgacatggttgcagccaccgcagcagccatcaatgacgatgcacccattcttaactttgatgcactatcgtccgatgtgacgacgggcttaccttccattgaacatctcacttttcccatcttgctcattgatggcaacccgagaaccggagagccacgtgtagtagtcgtcggcttttggagcaacgagaaggatggtgatgaggtagaaaaggaagctgagatggatgccatcttcttctttagttgatttgcaagagcttgaaggttcaacaaaggagattgtagtgcaaagttaaggggtttagtttgtgtttttatataaagttgaattattttgattggtttattgaaattatattcgatgcaaccacataaattggattacaaggattgcacctcatctatactcaaccactggtttcactccacgtgtcatgtcatctctatatcatagtgaaatgtttggatgtacaggatcggatatttgagtgtaaagt

SEQ ID NO: 42 - DNA, 629 bp, Artificial Sequence
  Synthetic Construct
caaaaatatccactaaaataatggtgaaaggataagatgataaagaatgtggtcccaataacgtcaatgtctacacgttgcgatgtctctttctttccttcttactatttttaatttttataatattaatattgccctaatcagtttatatctcactctgtgtaacttgtaatcaacagtcagcttctattcagtaggtgaaaatgcaaagggcaattggatctttcgtatcacatggaagcattgtgaagaatgcggttctgcaacacgttcgtttggtcaacccacttgcacaaactcttgttagacacgagtcggcgtctgctgcccgtatggaagaacatggttttgaaaaaactcaaatttcagatgttttgaaatccaaagggaaaagtgctgatggctcatggctttggtgcaccaccgaggattctgtgtatgaagcagtgaagtcgatgacacaacacaatgttggagcattggtggtggtgaaacctggggaggaaaagtcgattgctggaatcatcacagaaagagattacctcaggaaaatcattgttcagggaagatcatccaagtcaactaaggttggagatatcatgaccgaagagaacaagcttatcacagtc

SEQ ID NO: 43 - DNA, 453 bp, Artificial Sequence
  Synthetic Construct
cgcagcataatcatccaaatcaaccactttaaacacagccttctcatatctcgctttcgcctcttcatgcaacgccttagcaaaaccttcagcagttccagtctgtgttccaaaaaatatggtaaccttcttcttaccgtcatcaacctcttgctccacgtctctcttcggtacaacgatcttaggaggctctaattccttacctgacttcttaccagacgatctattccacaacacaaataaaaaacctattagaacagcagctgacgtcatcagcatcagcaactggttctccataagcatcgtcaacgtcaccggcatcgtcgatgtgttcgttgtgtctagtggtttggtgttaacaacggcggttatgagatcaaacggcgtcgtttgagtggagtctggttgcattccagttgaagaaaatggagcctcgaagtgatggttgaatgt

SEQ ID NO: 44 - DNA, 1208 bp, Artificial Sequence
  Synthetic Construct
tcaagaacatccacaacaagagcgctaaacacaacagccgcaaaatctctacctctagcatgaccataaacgctaccaaaaataatcccttcacccactcacccactataaaaagtaagcagttgcaatagacagaagcttaaatcaaacctcaaaagcagcagatagatatcaaacctctaaaaaggcagcaaatgtttatcatgcttattataaaaatctcctacccggattaaaccaccggtcaaaaacagaaacaaacatagaaataccagaaattcaagtccacctcatccagacaaaactgtctctaaaaaacatagtactttagacttacgtagcaaagtttagtacttgtagtccttgacatggagagactcggatgcaccttcactcaaaccagaaccgccctgatattttccaagggttgcctcagagttagccttgcaccttgcaagaaacgcagcctgggctttctgcacattttcttctttcccggaccaagccttgagggtgctttgttgtagggcccggccaaatgaaaatgacaacgaccatggcttcttaccctggtactggttcatggcgttgaggttaactgtagcctcttcctcactctgaccgcctgacaagaaaacaacggctgggactgcagctggcatggttcgctgcaaggcacggacggtgtactcagctacaacttctggtgcaacctttttggcatcagacccaggtgtaaccatgttgggtttcaaaagggtaccctcgagaagaacatggtggtcgttaagagctttgtagcaggcagctagaacacgctcagttacatcagcacacttgttgatgtcatggggtccgtccacgagaatctcgggctcaacgattgggaccaaaccgttctcttggcagatgatggcatacctagctagcccattggcgttctcattgatagcaagctgtgatggctcgtttgggccgattttcaagactgcacgccatttggcaaacctggcaccagctgaatagtactgagcacaacgttgggcgagaccgtctagaccttgggttgtggtctcaccgtttgttccagcaagctcaacggtacccttgtcaaccttgataccgggaaggactccggcttccttcataacatcaacaaatggcttacctgcagctgtcttctggtagagagtctcctcgaaaagaat

SEQ ID NO: 45 - DNA, 386 bp, Artificial Sequence
  Synthetic Construct
ggtatttctgaatttcttggaaatcactgatttctccacttgcaccaataagcgaatgtttgcccacttgctttaatcgctccacactcttgtagcgaatggtggatccgtatgaccctcccatgtcagctgccatgagaattccatccttgtatttgatcccaatgatggatgtacccgtcacgtatggatactgagttctctgagaatctccttcggatgataataaagtatccatctactgttgtttgaacacgttagtctgatcgttatcgtcaacctgtacggacaaaaacagaaataaaggaattgatggaacgcataagagtgaaaagaagatgaaggattaatgaaaaaatgaagagcccccttgtatttgatcccaa

SEQ ID NO: 46 - DNA, 1146 bp, Artificial Sequence
  Synthetic Construct
aaagaaattcatttttcacatagtgaagctattataatgactcactactatactagggttaatgaaacattaaaatataaaaataaaattttgtgattatatgtattaggtatatggtttgggcaaacaagtatatgttttcgggtaatcaggctaagtatcacgtaatctcattcttgtttcgtactgcagaaatctacaaaattgatcccatatacctggttccaggcccgaatacccgtcccattaactgttccgcatgtttcgggtttgcccatcaattgggatcggttcgggttcaattgccatacccaacgtcatgttcccatcaaaaaatattaatgacgtaacactgggaccaagttataaacgagtgacaaagctggtgctggtatagtttgccctcttatttcgtttttattaccaaaccaacatatttctagttataataatacaaagaaaacacacatcaggaatgattcaaacacattgtaggacaaacaacatgtcacacttgaacaaagtggactcatttccacttcttgccaaactttccatgcttgccaaactttccatgcttgaacttaccatggtgatgcccatagcctccgtagccatgtgacccataaccatgcccatagccatgagacacatgatgagttccatatgcagcagccccagcaagtgcagctgcaccaccagctaggagtcccataccatgtccattatgtccaccgtatccgctatgacctgaaggtgggtagccatgtgttggatatccactatgtgatgaatatccgccatatgtaggataccctgccggagggtatcctgtaggagggtaagtatgtccttgaggtgggtatactccatgttgtggagggtagtatccgccaccactatatccaccatgatgagagaagagtcctttgtctctattgtaatcgctacccatctttcttatccgagatgtatgcaacaagttgagtgtttgttgatctaagtttataaaagctttgattcaaattgcaagaaatttaggagggagagggagtgttatataggaaggtaagaagacctttaattgattcactcgttgtttaaatgcgtgttaatggagaataagacatgtcctaaagaataaag

SEQ ID NO: 47 - DNA, 368 bp, Artificial Sequence
  Synthetic Construct
gtttaatcattttgttattgatggagggtaataatgaaataagtaggtcgaagacttgattcgtttagaagtctattttggatatatttatatcggtttacattgacccctatcgccttttagaaagagagaaggtatgttatatgcatatttatgaaatgatgatgataatgatcaacataccatgaaaatcttcattgtcatcgacagtagaaaagaatatcataatatcttatgatcacatatatatccaagcttccttttcatatgttttgttttcatggcttcaccttcttcttttgacactttctgcttcatttgctccaccgtccaccaccatcatcaccaccaccaccacctctaatcaa

SEQ ID NO: 48 - DNA, 1596 bp, Artificial Sequence
  Synthetic Construct
aagggatgcgggatgtatccggagaaccaccaagtctattaataacgatcacaacctcatcttccaaatcaattcttttaacaccttctagtcgatcatcatgtcttgctgcaatggaaagtgtggctgcggctcaagctgctcttgcggtagtggttgcaagggatgcgggatgtatccggacatcgagacctccaacaccgccaccatgatcgtcgatggcgttgctccaaagaagatgtatgatgatggaagtgatgcgagctttgtaagtgaaggtggacatggctgcaagtgtggaagcaactgcaagtgtgatccttgcaactgttaaaaccaagatcataaaaataaattagcatcatcaatcttttgattgtgtgtatgtttgtagggtttgaaaaataaaaaaaaaagtaccaaaacccttgctcgtcgtaccttaacccgcttgggttggtacgggtgttggttttggtttgggtttatgttggtttcaatgctactataagtggtggtaattgtgtgttaagtagtatgattatggagtattggatgctttgttatgtatttatggagtgatgagtgatctatgagttttattaaacaaaaatgggcatgttgtatgtatgattaatagttaagatcgcataatttagagaaaagaaatatatacgttctaaaactttttagttccatctttatgaaaagaaatatattcttgtatcacttattattactattttttagtagtttaacaagctgtttggctaaaatagatatcttagaaagaaacaaagttaacatatttaaaaagtttttcaaacattttagaagtgatgtaatataaactttatcattgatttaaaacctaatcctacgacgagactaactgttacataatattaaggatttagactccattttgatagtgaaccacaccacttaacatcaacaataaataataataaattagtaaatagataagttccaaccatttagtgccacaattgattggatattgacatattgtgatatatgtcatgaggatgtagaaaataaggtatttttttttcatgtaacatgatatgcacaccaaaatacatacaaactttaaaaaaaaaaaaatctatgtaacttgttaggacaaagaggattggtgaaaccacttttggaaaccttagtctaattcacttctgtcattttggctaagtatagaagtactgaaaagaaaacaaagaactgctgaagagaagattagagaagtgctgaagaagaaacttcatcagaagttctgaagtccaaaccagacaagttcacttcagaagtactgaagacaaactactcatcagtagtttgtcattcatcagcacttgtaatagaatccttccaccgccttatgttttagtagtttaattgtttattattactttatcattttgtacaagttccaagactacagcctgttgtagtattgcttttgtgtaaagttttgaacaagttgtatgtatttgctggtctataaatagatggattcatcccactattttagatattgattttaattgaaaactgaaatccttcaaaaaaaaaaaa

SEQ ID NO: 49 - DNA, 1037 bp, Artificial Sequence
  Synthetic Construct
caacccaacccaacccaacactatctatctatctatctatctattgtttgttggctgatgagtgagtatatgcatatgcatatgcaagggaagggaagggaaagaagaagtgttgtcaaaaataataatactccgtaataataataataataatggcggcgtctttgggaaatgcgcagcttgttccttgtttgctaccggctactgctactacccaatcacccaaacaccacccatctccaactgttgcattcagatcacatttccatggcctcaccctctctttttctttttcttcttcttcttcttattgtttgaacaacagcagcagcagcaagacctcctcccttacaattgttgctgcaaaggtaaacaaagggtctgttcccccttctttcacacttaaagatcaagacggtaagagtgtgagcctctccaaattcaaaggcaagcctgttgtcgtctacttctaccctgctgatgaaacccctggctgcaccaaacaggcatgtgcattcagggactcgtatgaaaaattcaagaaagcaggcgcccaggttgttgggatcagcggtgatgatgcagaatcacataaggcgtttgcaaagaaatacagactcccatttacattgctgagtgatgagggtaacaaagtgagaaaagattggggagttccatctgatctttttggtacattgcctggccgacagacgtacgttcttgacaagaatggagttgttcagcttatctataacaaccagtttcagcctgagaagcacattgatgagaccttgaagttgcttcagagtctttagtttatttaataattaattaataatttaatcaaccaactaaacatgccatgccgtctatctatcttttcttaatcagttgtacaaatgatgagcaaacatatttattctgttatatgcaagtttccagagttgagtttatggttgtcaaataaaaaatacagtaatgtaccaaatatgaaacaataataacatttgtattaagattgatttttttaaaaaaaa

SEQ ID NO: 50 - DNA, 11830 bp, Artificial Sequence
  Synthetic Construct
aaaaaaaaaaaaaaaaaaaaaaaagaacctaaaagacctttattttcctcattctatactttatttacatgtaataaaatacctcaaaggattgtaaactttactaataaattgttttgattgaaatagaaaacaatcaatcccataatgaattagttaatgagtttaaataaactaatgaaaatcaagagttttgatttcattagaccgatgcccttagttaatcctataattcatatcaggataaagtgctctttttagcctaaatttttctccttgctatatttgcattttcctattcattttcctattcattttcattttcctattataagtattcgttttgatatgtcacttagtcatatcatttgaccaactgtaacttctttttttgaatatttgaacgattttgaaaagactcagaataaatactaatataaacatattcaataagttagttcatatcttgattttttattgagttttttcgaacgaggcaagatccggtaaatcggaaacaattaattaagaataaaaaactttttttatggaacagacatatcaatatgcgtggataatcccttttcttccacttccagttcctatgttaatagggttgggacttcttctttttccgacggcaacaaaaagtcttcgtcgtatgtgggcttttcagagtgttttattgttaagtatagtcatgattttttctatgaatctgtctattcagcaaataaatagcagttctgtctatcaatatgtatggtcttggattataaataatgatttttctttagaatttggatacttgatcgatccacttacttctattatgttaatattaatcactactgttggaattatggttctgatttatagtgataattatatgtctcatgatcaaggatatttgagattttttgcttatatgagttttttcagtacttccatgttgggattagttactagttcaaatttgatacaaatttatattttttgggaattggttggaatgtgttcgtatctattaataggattttggttcacacgacctgttgcagcaaaggcttgtcaaaaagcatttgtaactaatcgtgttggtgattttggtttattattaggcattttaggttcttattggataacggggagttttgaatttcgtgatttattccaaatattcaataacttgatttctaataatgaggtcaatcttttatttgttactttgtgcgctgttctattatttgccggtgcgattgctaaatctgcacaatttccccttcatgtatggttacctgatgcaatggaagggccgacccctatttcggcccttatacatgctgctacgatggtagcagcgggaatttttcttgtagctcgacttatgcctcttttcatagtcataccccacataatgaatttgatctctttgatagggataataacagtttttttcggagctactttagctcttgctcaaaaagacattaagaggggtttagcctattccacaatgtctcaattgggttatatgatgttagctctaggtatggggtcttatcgcagtgctttatttcatttgattactcatgcttattccaaagcattattgtttttaggatcgggatccgttattcactcaatggaaactcttgttggatattgtccaaaaaaaagtcagaatatggtgcttatgggaggtttaacaaaacatgtaccaataactaaaaattcttttttattaggtacactttctctttgtggtattccaccccttgcttgtttttggtccaaagatgaaattctgaatgatagttggttgtattcacctatttttgcaataattgcttggtctacggcaggattaaccgcattttatatgtgtcggatctatttacttacttttgaaggacatttaaacgttcattttcaaaattatagtggaaaaaggaatacccccctgtattcaatatctctatggggtaaagccggttcaaaaataagtaacaaaaacttgaattttcgtttggtaactttattaaaaaagaagaagaatggatgtccttcttttttttcaaataaagtatataaaatggatgaaaatgtaagaaatctgatccaaccctttctttctattccgaattttgacaataccaagacttctttgtatccttatgaatcggataatacgatgttattcccaatacttatattaattctatttactttgttcgttggattcttaggaattcctttcaataaagatgtggatatattatccaaatggttaactccgtctataaatcttttacatcaaaattcaaacaattcaatagattggtatgaattttgtgaagatgcagttttttcagttagtatagcctctttcggaatatatatagcattttttttatataaacccgtttattcatcttttcaaaatttggacttaattaattcatttgttaaaatgggtcctaaaagaattttttctgacaaaataaaaaatgctatatatgattggtcatacaatcggggttacatagatgccttttatggaacattcttcactgggggggttagaaaattggccaaattcactcatttttttgatagacgaataattgatggaattccaaatggagttggttttatgagtttctttgtagcagaggttattaaatcggtagggggtggacgtatttcttcttatttgttcttttatttttcttatgtgtcaatctttttattcatttattactttttaaatatttaagtcttttttaagtctttaattaagtctttaagaacaatccatttcatgaataaaatgtgaccaattatccaaccaacaaaactacttgttacaaatagcatcttgctgttgcatcgaaacataaaaatgttgactaatctcgctaacattgaacttggtaaaatgaaatgattcaataattgaaaaatgagattattcaggaatacacattgaatgctgagattacgcattgaatttctggtagtagatccataatcaaagaagtgtttgttattattgcagaagaaatgaaacaaaagatacggtagagctaggacagttattgtatgaggtctacccagtgctagatgcagaggcgcataatagatcgatatgaacattatgagctgtcccgtaataaaaccagttgttgctgataccttcttctcggctccttcttctccttcttccataacgtgagctcggagaaggaagagataggagggtcctatggagaatgtggtcagaaatccataatagagtccgaccacaacgaccgaattgattatcttcatgcataaggatactagattacctagtagaaaagatttcaaaatcatcacaaacctccctttttcttttctattgcaatttctggattattatatgatgattttgaaactttccatatatagaaaagaaatagaaagagatagactagaaatgacatctgttatgttaatgacaccaaaaggatattaaatgaatggaattgggatatggatggaatataatgaaatagagccactttgaggttccctatgaaatgaggcatggaggggagccactacgaagaagttccgggagttacgaaggaagattcgagctcatattggtcatgggttgagaacgggaatggaactctatgagatcgaatctcccgttgttcctcagtagctcagtggtagagcggtcggctgttaaccgattggtcgtaggttcgaatcctacttgaggagatttgaaaatcaccgtcaacaccccggtgtcggtccgggatactcctttgttccatagccctggggctatttacaactatccaattaagaattctcagatgtactagtactagcagtgcatcaaagatgcagtcatcgattctaccgagaggccacaattgccgcgagcaaacatattaatgacgaggaacgcgtttttgttatgctactaatataatacttgtacttgctctgctattctgccccatcctggctgaggaagagttacgggcgcgtaaaacaaaaaaatacgctgatgggggacgggcatactatgtgtaatgcttcactcatttacgataaataataaagaaaaggccattccatttcgacaaaagacccacacccaagttccatagcttttggtccgctatcccgatcatgattttcctacccccagagggaaaggcccttcccttttgggccggttgtgggcgaggagggattcgaacccccgacaccgtggttcgtagccacgtgctctaatcctctgagctacaggccccactgcgtctccactggatctgttcccggaagtaccctcaagaacctttgctctccccagccatttcgggttaagaagatgtcaaagcgcctttatctctactttatctctatttatctctactttatctctataagaacggcgcgttccgaggtgtgaagtgggagagaagggatgtcataattggggttttgaataagacgacccttttttttttttcatattgaaaaagtatgaagaacgggaggtgtgaagttttttatcatcctggcgtcgagctatttttccgcaggacctcccctacagtatcgtcaccgcagtagagtttaaccaccaagttcgggatggattggtgtggttcctctacgcctaggacaccagaatatcgaaccatgaacgaagaaaggcatgagagaaaatagtgattgcgaggccccaattcttgactgggggggacaccaaaggactctgcccttccatcccttggatagatagagagggagggcagagcttttggtttttcatgttgtcaaagagttgaacaatggttttttcgtgttgttaaagagttgaacaatgaaaatagatggcgagtgcccgatcgaattgatcgggtcatgtaggaacaaggttcaagtctaccggtctgttaggatgcctcagctgcatacatcactgcacttccacttgacacctatcgtaatgataaacggctcgtctcgccgtgaccttctcttgaattctcaaaacttctgtcgctccatccccgcaggggcagagaacccgtcgctgtctcggctgtgctaccggaggctctggggaagtcggaataggagagcactcatcttggggtgggcttactacttagatgctttcagcagttatccgctccgcacttggctacccagcgtttaccgtgggcacgataactggtacaccagaggtgcgtccttcccggtcctctcgtactagggaaaggtcctctcaatgctctaacgcccacaccggatatggaccgaactgtctcacgacgttctgaacccagctcacgtaccgctttaatgggcgaacagcccaacccttggaacatactacagccccaggtggcgaagagccgacatcgaggtgccaaaccttcccgtcgatgtgagctcttggggaagatcagcctgttatccctagagtaacttttatccgttgagcgacggcccttccactcggcaccgtcggatcactaaggccgactttcgtccctgctcgacgggtgggtcttgcagtcaagctcccttctgcctttgcactcgagggccaatctccgtccggcccgaggaaacctttgcacgcctccgttaccttttgggaggcctacgccccatagaaactgtctacctgagactgtcccttggcccgtacgtcctgacacaaggttagaattctagctcttccagagtggtatctcactgatggctcggacccccccggaaggaggccttcttcgccttccacctaagctgcgcaggaaaggcccaaagccaatcccagggaacagtgaagcttcatagggtctttctgtccaggtgcaggtagtccgcatcttcacagacatgtctatttcaccgagcctctctccgagacagtgcccagatcgttacgcctttcgtgcgggtcggaacttacccgacaaggaatttcgctaccttaggaccgttattgttacggccgccgttcaccggggcttcggtcgccggctcccctgtcatcaggtcaccaacttccttgaccttccggcactgggcaggcgtcagcccccatacatggtcttacgactttgcggagacctgtgtttttggtaaacagtcgcccgggcctggtcactgcgaccccctttgtgaggaggcaccccttctcccgaagttacggggctattttgccgagttccttagagagagttgtctcgcgcccctaggtattctctacctacccacctgtgtcggtttcgggtacaggtacccttttgttgaaggccgttcgagcttttcctgggagtatggcatgggttacttcagcgccgtagcgcctggtactcgaacattggctcgaggcattttctctaccccttcttaccctgaaaaagcagggtcaccttacgtccttgaaccgataaccatctttcggctaacctagcctcctccgtccctcgggaccaacaaggggtagtacaggaatattcacctgttgtccatcgactacgcctttcggcctgatcttaggccctgactcaccctccgtggacgaaccttgcggaggaacccttaggttttcggggcattggattctcaccaatgtttgcgttactcaagccgacattctcgcttccgcttcgtccactaccgctcgcgcgggtgcttcactctaaggcggaacgctcccctaccgatgtatttttacatcccacagcttcggcagatcgcttagccccgttcatcttcggcgcaagagcgctcgatcagtgagctattacgcactctttcaagggtggctgcttctaggcaaacctcctggctgtctctgcacccctacctcctttatcactgagcggtcatttaggggccttagctggtgatccgggctgtttccctctcgacgatgaagcttatcccccatcgtctcactggccgaccttgacccctgttattttgaggtcatatctagtattcagagtttgcctcgatttggtaccgctctcgcggcccgcaccgaaacagtgctttacccctagatgtccagtcaactgctgcgcctcaacgcatttcggggagaaccagctagctctgggttcgagtggcatttcacccctaaccacaactcatccgctgattcttcaacatcagtcggttcggacctccacttagtttcacccaagcttcatcctggtcatggatagatcacccaggttcgggtccataagcagtgacaattgccctatgaagactcgctttcgctacggctccggtgggttcccttaaccaagccactgcctatgagtcgccggctcattcttcaacaggcacgcggtcagagccccgggctcctcccactgcttgggagcttacggtttcatgttctatttcactccccgatgggggttcttttcacccttccctcacggtactacttcgctatcggtcacccaggagtatttagccttgcaaggtggtccttgctgattcacacgggattccacgtgccccatgctactcgggtcagagcgtaagctagtgatgctttcggctactggactttcgccatctagggtgcagcattctactgcttcgcctagcagcacgacgcttgtattgctctcccacaaccccgttttcacggtttaggctgctcccatttcgctcgccgctactacgggaatcgcttttgctttcttttcctctggctactaagatgtttcagttcgccaggttgtctcttgcctgcccatggattcagcagcagtttgaaaggttaacctattcgggaatctccggatctatgcttattttcaactccccgaagcatttcgtcgcttactacgcccttcctcgtctctgggtgcctaggtatccaccgtaagcctttcctcgtttgaacctcgcccttcattttaaggctatgccatcctaaggtgctgctaaatggatggatcttatcaacgtccatgaatgataaatcatagatcgaaccgccgaatcggaaaaattgggtgctatcatatagctttgtatcggctaagttcacgagttggagataagcggactcgaaccgctgacatccgccgcagggtaaaccaccgcctctcaggtcccccgactgattctaccatagaggccaacgatagacaataactcccccccgaacacagcttacaactttcatcgtactgtgctctccaaagagcaactcttctcaaaatctcactcaaaaggtgctgagttggaatcgcattctaattctaactaaggattcttgtggttccggaggatccagctacaggagaaccaggaacggagagctttccccccgccgactctttggccttaagaacgctggttttaagaatgagtgattgcccttctccgacccttactgcccaacctgagagcggacagctaatgcgttccacttattgaacagggttctatggtcggtccgtgacccctggatgccgaaggcgtccttggggtaatctcgtagttcctacggggtggagatgatggggtcggtccatggattttccttccttttgccgtatttcgctcaaagggttgaagggagatagtgcatcaagctgttcgcaagggccaacttgatcctcttccccagggatccccgataagggaaccctaggagagccgccgactccaactaccgtccatgtacgatccatactagatctgaccaactgcccatcctacctcctctacgttcttgacgttcttgacagcccatctttgtctcagtagagtctttcagtggcatgtttcggtcctcttccccattacttagaaaaagtgagccaccggttcaggtacaagatactatcattaccgcctggacaattagacatccaacccgtaatcgcaacgacccaattgcaagagcggagctctaccaactgagctatatccccccgagccaagtggagcatgcatgaaggagtcagacgcttcttctattcttttccctggcgcagctgggccatcctggacttgaaccagagacctcgcccgtgaagtaaatcatcgcacctacggtccaaccaattgggagagaatcaatagattccttttcgggagcgattcatccttcccgaacgcagcatacaactctccgttgtactgcgctctccaagtgtgcctgttccccccttcttccttaccatggcaagtctttgtgaaagaactccgatgagaagaaaaaagaaggcgttaagagaccctcctggcccaaccctagacactctaagatcctttttcaaacctgctcccatttcgagtcaagagatagataaatagacacatcccatttgatttcagctttctccagacctcggggaaaagcatgaaaaaaaaggctcgaatggtatgatccctccgtcaccccagaatgaaaggggtgatctcgtagttcttggtctgtgaagatgcgttgttaggtgctccattttattttcccattgaggccgaacctaaacctgtgctcgagagatagctgtccatacactgataagggatgtatggattctcgagaagagaggagccatggtggtcccccccggaccgcccggatcccacgagtgaatcgaaagttggatctacattggatctcacctgaatcgccccatctatcctcctgaggagaagtttggtttcaaactccggttcgaacaggaggagtacgccatgctaatgtgccttggatgatccacatctcagggtcaggcgccgatgagcacattgaactatccatgtggctgagagccctcacagcccaggcacaacgacgcaattatcaggggcgcgctctaccactgagctaatagcccgtcgtgcgagccccccaccgggggcccactatgccaaaagcgagagaaaccccatccctctctttccttttttcggccccatgtcaccacacaggcggcatggggacgtaaaaagggggatcctatcaacttgttccgacctgagataataagctcataagcttggtcttacttcactgtcgagaaacgaaagaagacttccatctccaagtttaactcagacgtagctcccttctttttgggtgtgaagcagtgtcaaaccaaaatacccaacaagcattagctctccctgaaaaggaggtgatccagccgcaccttccagtacggctaccttgttacgacttcactccagtcactagccctgccttcggcatccccctccttgcggttaaggtaacgacttcgggcatggccagctcccatagtgtgacgggcggtgtgtacaaggcccgggaacggattcaccgccgtatggctgaccggcgattactagcgattccggcttcatgcaggcgagttgcagcctgcaatccgaactgaggacgggtttttggagttagctcaccctcgcgggatcgcgaccctttgtcccggccattgtagcacgtgtgtcgcccagggcataaggggcatgatgacttgacgtcatcctcaccttcctccggcttatcaccggcagtctgttcagggttccaaactcaatgatggcaactaaacacgagggttgcgctcgttgcgggacttaacccaacaccttacggcacgagctgacgacagccatgcaccacctgtgtccgcgttcccgaaggcacccctctctttcaagaggattcgcggcatgtcaagccctggtaaggttcttcgctttgcatcgaattaaaccacatgctccaccgcttgtgcgggcccccgtcaattcctttgagtttcattcttgcgaacgtactccccaggcgggatacttaacgcgttagctacagcactgcacgggtcgatacgcacagcgcctagtatccatcgtttacggctaggactactggggtatctaatcccattcgctcccctagctttcgtctctcagtgtcagtgtcggcccagcagagtgctttcgccgttggtgttctttccgatctctacgcatttcaccgctccaccggaaattccctctgcccctaccgtactccagcttggtagtttccaccgcctgtccagagttgagccctgggatttgacggcggacttaaaaagccacctacagacgctttacgcccaatcattccggataacgcttgcatcctctgtattaccgcggctgctggcacagagttagccgatgcttattccccagataccgtcattgcttcttctccgggaaaagaagttcacgacccgtgggccttctacctccacgcggcattgctccgtcaggctttcgcccattgcggaaaattccccactgctgcctcccgtaggagtctgggccgtgtctcagtcccagtgtggctgatcatcctctcggaccagctactgatcatcgccttggtaagctattacctcaccaactagctaatcagacgcgagcccctcctcgggcggattcctccttttgctcctcagcctacggggtattagcagccgtttccagctgttgttcccctcccaagggcaggttcttacgcgttactcacccgtccgccactggaaacaccacttcccgtccgacttgcatgtgttaagcatgccgccagcgttcatcctgagccaggatcgaactctccatgagattcatagttgcattacttatagcttccttgttcgtagacaaagcggattcggaattgtctttcattccaaggcctaacttgtatccatgcgcttcatattcgcccggagttcgatcctggctcaggatg

SEQ ID NO: 51 - DNA, 6833 bp, Artificial Sequence
  Synthetic Construct
ggatcgtggcagcaaggccactctgccacttacaataccccgtcacgtatttaagtcgtctgcaaaggattctacccgtcgcttgatgggaattgtaattcaaggcggccaacgcggctcgtccgccgcgcaagcttagcctacgacacgtgcctttgggggccgaggcccctactgctggtcggcaaacaggcgacgggcacacgcgtcgcttctagcccggattctgacttagaggcgttcagtcataatccagcgcacggtagcttcgcgccactggcttttcaaccaagcgcgatgaccaattgtgcgaatcaacggttcctctcgtactaggttgaattactattgcgacactgtcatcagtagggtaaaactaacctgtctcacgacggtctaaacccagctcacgttccctattggtgggtgaacaatccaacacttggtgaattctgcttcacaatgataggaagagccgacatcgaaggatcaaaaagcaacgtcgctatgaacgcttggctgccacaagccagttatccctgtggtaacttttctgacacctctagcttcaaattccgaaggtctaaaggatcgttaggccacgctttcacggttcgtattcgtactggaaatcagaatcaaacgagcttttacccttctgttctacacgagatttctgttctcgttgagctcatcttaggacacctgcgttatcttttaacagatgtgccgccccagccaaactccccacctgacaatgtcttccgcccggatcgacccgccgaggcgggccttgggtccaaaaagaggggcgttgccccgcttccgattcacggaataagtaaaataacgttaaaagtagtggtatttcactttcgcccgagggctcccacttatactacacctctcaagtcatttcacaaagtcggactagagtcaagctcaacagggtcttctttccccgctgattctgccaagcccgttcccttggctgtggtttcgctggatagtagacagggacagtgggaatctcgttaatccattcatgcgcgtcactaattagatgacgaggcatttggctaccttaagagagtcatagttactcccgccgtttacccgcgcttggttgaatttcttcactttgacattcagagcactgggcagaaatcacattgcgttagcatccgcagggaccatcgcaatgctttgttttaattaaacagtcggattccccttgtccgtaccagttctgagttggctgttcgacgcccggggaaggcccccgaagaggccgttcccagtccgtcccccggccggcacgcggtgacccgctctcgccgcggaagcagctcgagcagtccaccgacagccgacgggttcgggacagggacccccgtgcccagccctcagagccaatccttttcccgaggttacggatccattttgccgacttcccttgcctacattgttccatcgaccagaggctgttcaccttggagacctgatgcggttatgagtacgaccgggcgtgggaggcactcggtcctccggattttcaagggccgccgggggcgcaccggacaccgcgcgacgtgcggtgctcttccagccgctggaccctacctccgactgagtcgtttccagggtgggcaggctgttaaacagaaaagataactcttcccgaggcccccgccgacgtctccggaatccctaacgttgccgtcagccgccacgtcccggttcaggaattttaacccgattccctttcgaagctcgcgtgaaacacgctatctgacgggcttcccccgtctcttaggatcgactaacccatgtgcaagtgccgttcacatggaacctttcccctcttcggccttcaaagttctcatttgaatatttgctactaccaccaagatccgcaccgacggccgctccgcccgggctcgcgcccaaggttttgcagcgaccgccgcgccctcctactcatcggggcctggcacttgcctcgacggccgggtgtaggtcgcgcgcttaagcgccatccattttcggggctagttgattcggcaggtgagttgttacacactccttagcggatttcgacttccatgaccaccgtcctgctgtcttaatcgaccaacaccctttgtgggttctaggttagcgcgcagtttggcaccgtaacccggcttccggttcatcccgcatcgccagttctgcttaccaaaaatggcccacttggagctctcgattccgtggcacggctcaacaaagcagccgcgccgtcctacctatttaaagtttgagaataggtcgagggcgttgcgcccccgatgcctctaatcattcgctttacccgatagaactcgcacccgggctccagctatcctgagggaaacttcggagggaaccagctactagacggttcgattagtctttcgcccctatacccaagtcagacgaacgatttgcacgtcagtatcgctgcgggcctccaccagagtttcctctggcttcgccccgctcaggcatagttcaccatctttcgggtcccgacaggcatgctcacactcgaacccttctcagaagatcaaggtcggtcggcggtgcaccccgcgagggggatcccaccaatcagcttccttgcgccttgcgggtttactcacccgttgactcgcacacatgtcagactccttggtccgtgttacaagacgggccgaatggggtgcccgcaggccggtgccaggagcacgcaggtgccgaagcacgccgtgacggcgcgcgctgccaaccacgatcggcacaacggcatctccacaggcatatcaacaacctgggctttggccgccgtaccaatccgcaccggtccacgccccgagccgatcggcggaccgacatacaccgttccacatccgaccgggacgcatcgccggcccccattcgcttccctcccgacaatttcaagcactctttgactctcttttcaaagtccttttcatctttccctcgcggtacttgtttgctatcggtctcacgccagtatttagccttggacggaatttaccgccctattggggctgcattcccaaacaacccgactcgcagacagcgcctcgtggtgcaacagggtccgggcacgacggggctctcaccctctctggcgcccccttccaggggacttgggcccggtccgccgctgaggacgcttctccagactacaattcggacgacgaagccgaccgattttcaagctgggctgttcccggttcgctcgccgttactaagggaatccttgtaagtttcttttcctccgcttattgatatgcttaaactcagcgggtagtcccgcctgacctggggtcgcgatcgaagcatcatcgcaagacaacgcgttagggtactttaagagtatctacaacaaggaatgaaaacgcacgacacgagacgactgtgttatcaaccaccactagccgtgcgtccctcttgatgagactcccatttaggccaaccacaccatgggcacgggagaccaatctccgccccaaccaaagtagtcccttgatgggaatgtttggtgggggcgacgtgatgcgtgacgcccaggcagacgtgccctcaaccggatggcttcgggcgcaacttgcgttcaaaaactcgatggttcacgggattctgcaattcacaccaagtatcgcattttgctacgttcttcatcgatgcgtgagccgagatatccgttgccgagagtcgttttaggtttaacaaagatgccacatacaacacgcacaccgcgaacggggcgcaatagcacgggccctttaagtttagttttccttggcacgtaccgtgccgggggtttgttatcgtgtctccatgaaatccatgatgccccaaatgtttggcgcaagcatagacacacgtcgacaaggcctcacaaagatcgaaacgtaagcttcggtccccgcttagccggtgtttttacatgttcacgggtcgttctgctatgcagggttcgacaatgatccttccgcaggttcacctacggaaaccttgttacgacttctccttcctctaaatgataaggttcagtggaattctcgcgacgtcgcaggcagcgaaccgcccacgtcgccacgatcctaacacttcaccggaccattcaatcggtaggagcgacgggcggtgtgtacaaagggcagggacgtagtcaacgcgagctgatgactcgcgcttactaggaattcctcgtttaagaccaacaattgcaatgatctatccccatcacgatgaaatttcaaagatttcccgggcctgtcggccaaggctatatactcgttgaatacatcagtgtagcgcgcgtgcggcccagaacatctaagggcatcacagacctgttattgcctcaaacttccgtggcctaaaaggccatagtccctctaagaagctggccgtggagggatacctccacatagctagttagcaggctgaggtctcgttcgttaacggaattaaccagacaaatcgctccaccaactaagaacggccatgcaccaccacccatagaatcaagaaagagctctcagtctgtcaatccttactatgtctggacctggtaagtttccccgtgttgagtcaaattaagccgcaggctccactcctggtggtgcccttccgtcaattcctttaagtttcagccttgcgaccatactccccccggaacccaaaaactttgatttctcataaggtgccagcggagtcctaaaagcaacatccgctgatccctggtcggcatcgtttatggttgagactaggacggtatctgatcgtcttcgagcccccaactttcgttcttgattaatgaaaacatccttggcaaatgctttcgcagttgttcgtctttcataaatccaagaatttcacctctgactatgaaatacgaatgcccccgactgtccctgttaatcattactccgatcccgaaggccaacgtaataggaccgaaatcctatgatgttatcccatgctaatgtatacagagcgtaggcttgctttgagcactctaatttcttcaaagtaacaacgccggaggcacgacccgcccagttaagggaaggagcgcatcgccgacagaagggacgagtaaaccggtgcacaccagaaggcggaccggccgacccaacccaaagtccaactacgagctttttaactgcaacaacttaaatatacgctattggagctggaattaccgcggctgctggcaccagacttgccctccaatggatcctcgttaagggatttagattgtactcattccaattaccagactcgtatgagcccggtattgttatttattgtcactacctccccgtgtcaggattgggtaatttgcgcgcctgctgccttccttggatgtggtagccgtttctcaggctccctctccggaatcgaaccctaattctccgtcacccgtcaccaccatagtaggccactatcctaccatcgaaagttgatagggcagaaatttgaatgatgcgtcgccggcacgagggccgtgcgatccgtcgagttatcatgaatcatcgcagcaacgggcaaagcccgcgtcgaccttttatctaataaatgcatcccttccagaagtcggggtttgttgcacgtattagctctagaattactacggttatccgagtagcaagataccatcaaacaaactataactgatttaatgagccattcgcagtttcacagtctgaatttgttcatacttacacatgcatggcttaatctttgagacaagcatatgactactggcaggatcaaccaggtagcattcctcttttgacaatacccacccgcggtacgagccatacgggctaggcagtcattttaagagaagcgcgaacgctaaacgaaggcagatagaggatcccaagggaccccatgccaacatcaggttccgtatccaagataacaagcaacgctcaatgtgccacagcatcaatgcaagcattaacgatgtggtgcaaagagacagcttcaaggttcatccaatgccatgttagtgacacaagacgaaagcaactttgatcagccaaagacaataccatagcttaggtaggcaacacaggaaacccacagtatgcctaagcgaaaaacgcctaaacaaacaattgaagaggtaagccaggtggaagttgttgcgcaagcaaagagccaaccacccgtaacaaaccaaacaccactcatgcacctttacggttggcattcccaaagaccacaatgctaatagacacccaccataagcctagaggccaaggaagcaactaaaagccaagcgatccctagaatgtaccgggcgatgcgaaaacaatatcaaaggcaacaaggaaggcagcaaggaaggcagcaactaaggcagcaactaaggcaacagatttgccccgcactttgcatcaccagtatataaacaactcaagagcaatgacgttagagccttagacgatgggcaccatgcggtgttgcatccctttgtcctcagcaatcctcactaccctaacttagtttcaatacccatgatgcaaaatattgtgatgcttgaaatccaaacatcataggaaggcaagctttgaggatatggggttcgatgcatattactcatggcagacgactctatgggacgaatgatgaatttggatgcaccagggttcctgctctcagcttacccatctatgatatctggataacaagcatcacaacagccggacaaatctcgataagggacgttcttgaaagggaccagcgatgggcaccgctgccagaaggcacccatcgctgaa

SEQ ID NO: 52 - DNA, 922 bp, Artificial Sequence
  Synthetic Construct
gctatgcttatgaaatgacaaaaaataaataaaatgaaatatctaggggtaattctggtaaacaacaatatattatctttatagacattgccttatctcttacaagaggtgaatgggaacctcaagtttcttttaaattcaagattttgcaggccatcttttttcatacccatcaaaatcttgaaatcatcacatattattattattttaccataaaatatctctgcaaatatgtcttctgttttgaattgtactcctcttacctcttccacttcccctaccaaaccagggctaattggtggaactggtccaaaagtaactttcattaacgtttcgaataatcagaagtcaaaatttgaaagatttccagctttagaagtcaaggcttcctctgatggtacaaaagccaccacggcgtcaaaaagcttagtttgtgaatcgtgcgagggaaatggtataatacaatgttcacaatgcaaaggaactggagtgaacacagaagatcacttcaatggaaggttcaaagctggtggattgtgttggctctgcagaggtaaaaaggagatcctctgtggtggttgcaatggagctggatacgcgggtggattcatgagcaccgcggatgaatagaatacatgatcttgaaatggtgctcgttacgtcaggttgtttgtttgtatcgtttgttgttgactagcaactataaacattgagtatgttttgtttaattttgttcgattttgtatttcaaactgttaaagttgttaagttgcatttatagactctacttcttaaagctaaactcaagggaataattgtcaagttgcattctaacatcacgagtgtgggtggtcttttgtctatgaatgcatcagggtagccaagctttgtttgacgttaaaaaacacgcattaaaaactaa

SEQ ID NO: 53 - DNA, 1520 bp, Artificial Sequence
  Synthetic Construct
tttttttttctttatcaaaaacgattcgtccttgttattgaaatacaatgatattgatgagtgggtgtaacaatattacaattgaaaactctagttaaaactgaagagaataaaactattgtttaccatttgaggtttttgagtctcgtgtgcaaaatcccacgagtacaaaattcgtgtggttgatgctaaatctgtgatgacatactgatttattctaaatcttgtttgccactcatcaggtttaccttcttagaattattttgcgtattgttcatcgaatcacacacacccatctctatactcttatatgttcatgtaaaattgattcaaaacagggagatgttagatcctacaacgccaaatatagaaatacaaggaataatgctttgaatttgcatgcgttatttttttgacaaactcaactcaagacaaatgctaagagtatataggagtttgttggaagatcaaacatacataagcacaaaaagacacacataactacttaaccataatgagggaatacgaagaccaaaggagacggtagtaagttttattatggcgaccaaagggtgtagtgatagctttattgtggtgtgtttacgcattgcatcaatttcctccatctctctttccctcaatgctaaccttggctgcctcaagcctggaactcgccgcatcccatgatacaagcttatcgagaaaagttgctatatagtcgggcctgaggttcttataatccaggtagtatgcatgctcccaaacatctaaggtgaggagtggatagtaatccaacacaagaggattaattgcatttgaggtcttcagcaccacaagcttcttatgctctttgtcatatacaagccaagtccaaccagaaccaaagtgagtagcggcagctaacttaaattgcttaattagctcttcaaatgttccgaactctcggataattaagtctaaaagttccccggaaggctttccaccaccaccaggtttcatagattcccagaaaaattcatggttccaaacctgtgcagcattgttgaaggagggaagaatatcacccttattatatgaagctaatattatgctttctaatgacttttcatctaattcagttccctcaatttgcttgtttagattgttgacataacctctgtggtgttttccccaatgatattccaatgtctctttgctcatatgtggttccagagcatccaatgcataaggaggtggcttcaaatctatgctggctgatattgtcccgcaaacagattttctagtacgcggtctctgtgacgagtgtatgctctgaggtaatctacgaaattctggagcagaaagaaaattggaggttagatgagcagccattgtgctaacggacgccattgttattattattatgtttctgcagcagagcttgtaacaggaggatgaggatagttaggatgtggatggctatgaaagacctgtaattatttgatagtacaactacgtacatgcatggttcatgttgttat

SEQ ID NO: 54 - DNA, 797 bp, Artificial Sequence
  Synthetic Construct
taacaaaaagcacactgagcggaaccaaaaaccgtcatactcgacaaccgtccaaggtgtggttgactcacgaggtgtctttacggacatctgcattggctaccctggctcgatgtctgacgacaaaatcttggaaaaatcagctttgtcccagagattcaatatgggccaattgaaaaacatttcagtagtgggtgggtccagctacccactcctggactggcttttggtgccatatacgcaccaaaaccatacatggagtcaacattcctttaatgagaaaattggtgaggtacaaatgatagcaaaagatgctttcatgaggctaaaagggagatgggcatgtttgcaaaaaaggacagaagtcaagcttcaagatttgcctgtggttctaggggcatgttgtgttttgcacaacatttgtgagatcaacaatgagaccatggatgatggtttgaagtttgatttgtttgatgacgagattgttgttggggaaaatgctaggggtaaatcgttgaatgcggtacaagttagagatggcattgctcataatttattgcatcatagccatggaagtactggctttagatgattataggattatacaaaggtttttttatgcttgaaataatttttttggggtgttatgaaagaataattgcaagattaatgtaatatatcgttttttgcttgtaagttcaagtttatcatatgtactactcttaataaataataatatgaagttataaacttatatgttgtttcctttgtaaaaagaagtaaatcatccattaatt

SEQ ID NO: 55 - DNA, 765 bp, Artificial Sequence
  Synthetic Construct
aacaactccaattctcaaacttaaaaaaatgatgatgatgatgatcaacatcttcttcttcatctgtttcatcatcccaatcattcgaatcaagtccgatctactcaaatctttaaaaaattccatcaacatgttccggtactacagccatcacgaagagattaacgaatgtgtgttggatcttccagctattcgattcgaagatctacagaatcatcgtccaagatctatcgaccgcatgtgtttcgtatgctccaaagattacgacaaggatgatgtgctttgccagctcagtcgatgtggtgacatcttccactccaactgtgttggaaaattgattaaccagaaacaaacttcttgtccattctgttgcactccattcttctctggcctttcgactgtgccctgcgagtgaggtttcgaaaagcggtattgtatggtttgttagttacccgtagaattagtgagcgtgtgagttaggagtacgttctataaaagattatctgtattctaatatttatggtgaaacagtgctatcttttttgtatgttattgttagaagttataatcaagaagtttagaaggcttatgggttatctcggatgtgaaggcttatgggtttctttggaataataataggaatacaaatgttaattaatgtacttgggaagaatggtgtaattgtgatgtgatgtaatggccgtctagttcctcgccttgaggatcttgcggctctttagttttgttttaatgaagttcaatttt

SEQ ID NO: 56 - DNA, 3449 bp, Artificial Sequence
  Synthetic Construct
gccgccactgtaacataacagcataagttctgattgggtgcagcagtgtctggacatctatatatactagcctatagaatcaacattctgaacaagtaaataacatcacagaatattctctcctgtctacgatcaaatatgaatcgttacttgatcactctgcttattttattgattgttagagcggtcaacagccgtcaactggctgatgaccttattcggttgccgtcggagaaacttggtttctctgacgacgactctgttggtacccgatgggccatcttgattgctggatctaatggttattggaattacagacatcaggcagatgtatgtcatgcgtatcaaatcttgaaaacaggtggtgttaaggaggaaaacatcgttgtattcatgtacgatgatatcgcgaataactggcaaaatccgaggaaaggaaccattattaacagcccggatggtgatgatgtgtatcatggagtaccaaaggattacactgggaacgatgttaatgttgacaactttttcgccgttttgcttggagacaaaactaaagtcaaaggtgggagcggaaaggttgtagatagcggtccaaatgatcgcatcttcgtttactacactgatcatggtggccctggtgtgctcgggatgccaacttatccttatctgtatgcaaatgatcttattgaggtcttgaagaaaaagcatgcttctgggacatacaaaagcttggtattttaccttgaagcttgtgaggctggaagcatatttgaagggcttcttcctgaaggattgaatatctatgcaaccacagcctctggcccggatgagagcagttggggtacttattgccctggggagtatcctagcccgccgcttgagtacgacacatgtttgggtgacttgtacagtgttgcttggatggaagattgcgatgtacacaacttgcgaacagaaaccatcaagcagcagtataaactggttaaggaaagaacctcaaatgaaaactcttattatggatctcatgtgatgcaatatggggacatgccactaagcaaagataatctatacacgtatatgggtacgaaccctgcaaacgaaaacttcactttcattgaagaaaactccctgtcaccatcaccaaaagccgttaaccagcgtgacgctgatcttctacatttctggcataagtaccgaaaggctccagaaggttctgatatgaagactgaagctcagaaaaagtttgttgaagccatgtctcatagaatgcacatagacagcagcatccaactcattggcaaggttttgtttggaatggagaagggtcccgaggtgctaaacaccgttagatccggagggaaacctgtagttgatgactggacctgccttaaaacatttgtgaggacatttgagacacattgtggatccttatcgcaatatggcatgaaacacatgcgatccattgctaatctgtgcaactctggagtcacaaatgagcagatggctgatgcatcatcacaagcatgcaccaccttcccatcaaacccatggagttcacttagcaacggtttcacagcgtaaggttctttgtccttcagagactgtgggcccgtaatttgtcataatatcatatgtttgtggtttctgaaaccttctttcagcaatatgttgcatcattgtatataaagcctgaagcttatgcttatcgtagtatttaggtccacaaaacttggtatctatatatgtatatcgttcgcttgtaatagtgaatcaatgatatgaaaatcatgtaagatctgtatattagatataaattgcagtctgtatcacagttaaaacaatacgtacagctacttatgttattctcactgccctacgcgtaaaattgattctaaaccaaaagattgcatttataaagataatcaatggcttcaacccctgctgcaactttcggttttgatcatctcaataacccaaaaatttatgagcaacaagtgacaaaggttccaaagtccccggagaaaatttcctcgcgaataggtaacagattgaggaaggttctccgttataaatacagttttgaatgtgaagtttcttcatgaattcctccgttatgtcggacctcccaaatgtagcagggtgggggccacctctagaccaatcaacccaagtcaagctccggtttgctattaaatcaggagcctgtattgtcaacagggttgggaaataatgttcgtccacgtaacaagctggcctgcaaaacgcttcaaatttgggataataagtcgtatcggagatgatgactgatgcaagatgccggttaacttcaaaccattgggatccttttctccattgggagatgttgacttctggcaacatgttgtggttgtagcgtcccctaccgacgggcccagggtcatcaaaggctcccatgaagctgtgtttggagttggtaatgtaatgataggttgtagtgaagttgtagagtggaatgcatgattcggatacaagaacaaagtattcgttggagaaatcaagaagggcgtttgcgagcagtcgtctctctgcatcacacatactcattcgaccccattgtgctacctgactcgggatttgcctcccataaaaaacagatccgggcgaaaacttagcttcatatgaaggaagtgaatgaatgtaaaccgaataaaatccctcatgacccttgaagaacctctcccacaaagaagccaaaggcaatgggcctttagtcaagaacataaaagcaatctttgggactcttgtaaatggataactcttcaactgtggcacaaaagaagccctccaaaagagttcttcatcactcattgcatgcaacaaactggaaggaggcttcatccaatgatcaagactgctcttttgttgctctttcacacatggttgtaaaaccggaagcgatgaagctaatgtagtagtaacttcggtttttcgaatcatatatataacaaataaacagattgaaaaacataaaaccagaaacaagaacagcaactgaaccagtcttggtggtaacactctagattggtttggtttggttacagttacagcagtttggtctttaccttcctccatgatgaataataatcataaactgtatgttgataacacaaaaagattagtatcttttagtgggtatgttgaattataggtgcttagatcctttgacaaccttcttgcagccatagtgagtgtttgatctgtgtccaatgaaagaagttaaatgattaaaggagtcattgtgagttataaaaagaagctagaagaagatggtgaaagagacatgcgtccacaaaggtggggagacatcgagacgaggaatataatatcagacaagcaaaagatgttgttaaacaagctgtgaaggtgggagagagatccaaggaattaatgtggtctgtctacatctgagg

SEQ ID NO: 57 - DNA, 222 bp, Artificial Sequence
  Synthetic Construct
aaattttaaaacaaaaacatatataaaaaaaaaggatttgagcgtgtggcaacaagtgtaagttatccagttgtgatgctgaccaatcacctccacaaaaacactccttacacttaaccttatcaaccaccactctgtgtgtgtgtgtgtgtcttgtgccacccacatcatcaacatgtccacaaccacagcaggtgcaggtccagccacagccttctccac

SEQ ID NO: 58 - DNA, 868 bp, Artificial Sequence
  Synthetic Construct
caaaaacatgattccaggaactgctggtgggattcttttcttgaggagtttgagagtgtaatccgcgacttgttgaggtgtggctttgtccttgcagtcggctccaggtgtaaccatgctcggtttcaaaaggattccttcaaacaacacattgttctcagctaagtaaaagaacacctcagcccacactttctttgcaacctcaaacgtcgtatcgattccatgttcaccgtctagtaagatctcgggctcaacaatggggaccaacccattgtcttgagaaattgcagcgtaacgggctagaccccatgctgcttccttgaccgcgagggccgatggcccgttaggaatgctcacaactgtacgccatttagcgaaacgcgcaccctgttggtagtaagctgcagagcgcgaggcaagcccgtcaagaccttggcaccatgactcgtcattcgagccggccaatggaactagacccttgtcgactttgataccaggtacaatcccctgttcaacaaggatgtccacaatctttttgccatcaacggtcgattggtacaacgtctcctcaaagaggatagcacccgagatgtagttgccaaggcccggtggggtcacaagtagggttcggtaagcctggcggttagcctcggtgttctcaagaccgatagaagccaaccgctttccacaagtggcatttgactcatccatggctaggataccgcggcccggtgatgctacggttttcgcagttttaacaagctcatctgcataggaagcacggacgctcagtgcgttctggctgacccgcaccacggagaccgcgggttggcgaatggactgacccttaacccaatcagacttatca

SEQ ID NO: 59 - DNA, 1557 bp, Artificial Sequence
  Synthetic Construct
tttgtgtgtgttttgtgtgtgtgtgtgtgtgtgtggagaagatgagggggagagtgggtgttgtgttatccggtttttttgtgtgatgagaaatgataataataatattgggagttctaattcgatttggggttcgtgtcctgttgaatttcgttgccctatttctaaagaactcatgagagaccctgttattctctctactggtcagacttatgatagggcttttatccagaaatggttaaaagctgggaacaggatatgtcccaagacacaacaagtactttcacacactattttgactccaaataatttggttagagatatgataacacaatggtgcaagaatcgcgggcttcagtttcctggcccgattcagtattcggatcaagacgggctaacagaagccgatcgggatcttttcttatcgcttttgaagaaaatgcagtctacacaatctgagcagaaagaagctgcaaggactttgaggtcgttgacgaaaagaatgccttcgtttcgtgcgttgtttggtgaatctcttgagtccatacctcagttgttgaccccattgtgccaaaccaagtcccaaaccgaaatccaccctgatcttcaagaagatctgattacgactcttttgaatctgtcgattcatgataataataagaaacttgtggcagaaacaccgatggtgattcctctgttgctagatgcgttaagatttgggactattgagactagaacaaatgcagctgcaacgcttttcactttatcagctcttgattcgaataaatcgctgattgggaaagcaggcgctttgaaaccgttgattgatcttttggaagagggacatccactggcgatgaaagatgttgcttctgccatctttaatctgtgtattcaacatgaaaataaagctagagctgtgagggatggtgctgtaagtgcacttttgaataaaattaagaacagggttcatgtcgacgagttattggcgattctcgccatgctttccagcaaccagaaagctgttgaagaaatgggagatcttggggctgtttcttgtttgctgactttaataaaagaaacaaattgcgcccgaaataaggagaattgtgttgctgttttatacaccatttgttgctatgatcggacgaaattgaaggaaataagtgaagaagagagcaagtatggcactttatcacaagttgcacaaaatggtacttcacgggccaaacgaaaagccagtggcatactcgacaggttcaatcgtgccatcaacttgacccacaccgcttaaacatgctccctaaaataatctgtaaattcgacatctcttataccattccaggtttgagcaacatttataactttgtttcgcttgttttcactcaattttgtgaattcttgtctcaggtttgagcttattatagccattgtgaataattactatattactgcatatcctcagtataatactattttgaataaattgtacatttgaaataatgaagtataatgttgatggttttaaattattcaatagcagtcagggcaaccaag

SEQ ID NO: 60 - DNA, 3338 bp, Artificial Sequence
  Synthetic Construct
gttaaactgaaacgcccaagctcaaaactatataaacaaattttcaagtaccatgtccatatgtatttgaaaattaaaattgtaataacattaacaaagtgacaatattctttaagactacaacttataaacattttatcgtttgcccttttttatggtaactagtttaaaaacccaaattttttgtttttataaagacggatttctttaaccctatcatctatgagaattgaaccctgtacttccaaatctagttgttcctaaacactttgtttttgtcaatggccacgaacacattgacgacccatcaatatatagttaacatggtgagcaaaacaaactattgtttctttagtttaataaacttttaaaattagaatataaaaaagtagtatagtttctaatgttaatacacctcctaaaatttaataaaaaaatatcaaaacatattttctttgtagataaaacatacacatgtgaaggctacattagctaatacataagtacgcatgtgcattaaacaccacctaagttcaagctacagaaaaggagctctaagtgagtcaagttcgagctctctttggttaaacgagttgaactcaggtttagatggaatcagtttccttagtacacgacgaatttggttcatttcaattatcaaatgactaacttcatcctaattagcttatacattagctgatttgggttatgacttatttccaattggataaatgggtcaaatacaaatatttagttagaaagaaaatagaaaaataagttgaatgagcgatcattcggtgcaaaagatataacaatattaaagtatcaagagaatcccttccaacatgtttgggtgaaaaatattatccgttttcacctcacccttttggcatgctacccaacccgcccgctctgtctacttttccacctttaatatgtcaaatttctcttaataactaataaccaacctcgttaagtaaagtttggaaccggttactttgccagccatgcgagagatgggggagcatgatcatcataacgggccgataagaaatctccctcttgtgtaactacagaaaacaaaaaaaaaaggcactctccctacgcaggcattatcctgttcaggttctaagggactttctcagcctgaaactgccgcagtcaccccaccaccaccacccaaaaagggacggtagctcggtgctgcaaggagattcagacacccctacgctgaggattttgaacttcagtttatgctaggccgagtcttgcacacagtccaaagtgatcctcgatttcggcatgctacctttatttcttcttagcattgatgtacgattccggcaagtagatctcaccaccagcctcgccatgttgttcaccactaatggtcttttttgccgcctgaaactcagcactcatagcatccatcccttgttgaacagcctcttcagcactcccatacccatgttcttcagcatacttcctcacatcctcagtaatcttcattgaacaaaacttgggtccacacatggaacaaaaatgtgccacttttgcaccatcagaaggtaatgtttcatcatggaatgatgtggcggtcatcgggtccaatgacaaagcaaactgatccaaccacctgaactcaaagcgggctttgctaagtgcatcatcccaggcttgagcatgtgggtgacctttggctaagtcggctgcatgggctgctattttgtatgatatgacaccggttttgacatcatcacggtttggtagaccgaggtgttcttttggtgtgacataacagaggagtgcagttcccaaggctccaatgttggctgcaccgatggctgaggttatgtgatcatatcctggtgcgatatcagtggttaacggtccgagtgtgtagaaaggtgcttcattgcaccattcgagctgcttttgcatgttttcggggattttatgcattgggatatgtcccggcccttcgttcatgacctgcacatctttctcccaggctctacgggttaattcaccttgagtcagtaactcggcaaactgagcggtatcattagcatcataaatcgatccaggcctcaacccgtcaccaatagataaagctatatcatactgattacaaatatcaagaatatcatcccagtgttcataagcaaaattctccttatggtaggccaaacaccattttgcatgaattgacccgcctcgagaaactatgcccgtcatccgcttagctgtcaacgggatatagcgtagtaaaacaccagcatgaatagtgaagtaatcaaccccctgctcagcctgctcaatcaatgtctcgcggaaaacttcccaagtcaaattttcagcaatgccgttgactttttctagcgcttggtaaatgggaactgtcccaacgggcactgcagaattacgaagaatccattcacgggtttcgtggatgtgtcggcccgtggataggtccatgatggtatcagcgccccacattgtggcccactgaagtttgtggacttcttcttctattgaacttacaactgcagagttgccaatgttggcattgactttgaccaagaagttccttccaacaatcatgggctccaattctagatgctttttgttggaaggaatgattgcacggccacgagcgacttctgatctcacgaattctgggtcgagattctcacgggttgcacagtagagcatttcttcagttatgattccatgcttagcatagtgcatttgagtgtatcttggtgggctaagtttttccctcctgtcaatccaatccttgcgtaatttcgggagtccaacttgagggcttatgttttgtggaccactggtgtcatatgtgtcaaaatgcggctcatcccctgcaaggtggacacgtcgaaatggaactttaagaacattaccagattcctcatgaacaacttccctgtattcctttgtgctttgtgggaaacattgttcaaatgaaggaagtggaagaaactcaggtgcagttgggtcaactgtgctcttcctttgcttagtcttctccacattggttgacggcgggtcaaaggtcaacgtggcccgaggcaccctgatgcatgatggatgtacttccttcttgcataagttaggagtattggtggtcacatcgaaaacaggcaagaaaacagactttgaaacctttgagagtctatggttggcattttgacgcataacagatgtaaaattagcagtatatattgatgccatctttgtatatgtctatcaaaagaatccgctgatctcgaaagcgcagtagtgagtgatcaatgatgtatccccgaggcaccctg

SEQ ID NO: 61 - DNA, 823 bp, Artificial Sequence
  Synthetic Construct
tctacatcttaccctcctcagactctaccttaacttttgctattgatggaattaactgagtatgatgatgaagccaagctacaccatccccaacaccctgcatagcccagtcaacatagcttcctcactcaattagagtatgaaccaattggagtatgaacactaaatcatctcatacaatgaaataatgcagaaagtacaactcaatctagcatgaaagtaatggatttggcatactcgattatatttataaagaataaataattatacaattttagaattcaatattggaaatcatgaacatcctgttctaaggcttcttctaacaaaaactcagattgatccatcaaatcagggcttaatctgaacataagaacacaaaattccatctgactaagtgcaccatctccatcaaaatcaccttccctcaacatgctcaaaagatcagcatcagacaaatcctgaagacccaaaagcgacgaattcttcttcagactgtcgaaagtgatcacgcctttatcgcgatccatcagcaactgaaacccgttgcacagttcttcgactagaccatcaccgccaagtttatccgccattagaggtaagaaatcttggaattgagattggttttttgtttccattgattgaaatgaacaaaaatgtatcagttaagtgttgaagattggttggatattggtgagaagatatctagagggatgattatataaccatgttgtaaggaaggtgtatacaggttccaggaagggaagaaaagccatcggatgacagcagatttgagtattgttgcttccacgaaggacttgcg

SEQ ID NO: 62 - DNA, 263 bp, Artificial Sequence
  Synthetic Construct
gtgattggatgtgagtctagtctatgtgaagaactaaaatatcatcgatctcctccgctaaaaagtaaaaccagcgttgaagcaagcaaagataggcgtatccttcactctgcacacaactcacatccaatcacacccaaaaccatgatcacaacgctaacaccaatcctcaaacaatatatatccaatcaatttatataaatccactccccaatctaacaataataataataataatacacgtcatctcaaccctaacccta

SEQ ID NO: 63 - DNA, 523 bp, Artificial Sequence
  Synthetic Construct
tttttttttttttttttttttttattttaattctgttagttaattttttatggtaaatttttacttattaaatatttattatttgagatatattataacatacttctgagtgttaaatttttaatattacttctcttttagaattttaaaaaatatgtactttttgatttaaaacttatcttttaagtcatttaggtctatatttttacaaatttatttaaaagtttggacgttatagctcttttaattaaattaacttatttaacaactagatataaaaaaatcgtattcttataaaatctatgtttagttttattttaacttttttacgttaataaatactttttagcataattcttttttttttcgagaaaatttcttctgccatttagttaattaaccctgatacaaaaggtatgacaagataggtaaactttttttttaattttttcttttgttttcactactttttttattaactatagattttttattatttaaattctttttatttactatattt

SEQ ID NO: 64 - DNA, 928 bp, Artificial Sequence
  Synthetic Construct
aaggtaaatcgaggaaaaactggggccattagagcctgagacaaccataatatccaatcagtctctcatctacacgttgcagatcctacagaggaagggattgaatcgtctgtatgaagcgattctttccagagctcacttcctgcttcagtggtgcacctgcggttcataccatcgccgccgacgaggctcctacccgtcgccggcgcatggcgcaaggtaaatcgaggaaaaactggcgcccgaagctcgcggcgatcgacgaagacgcttcggtccgtgacgcgcgaaaggcgaatgctcaatccggttctgggtttagaagtgagaagaaaccgttgattagaagtaaattggcggggaaaattcggtcggattcgtatgagtttgatcaccggaaatcgagtgatgcattcgtgataccagccttttcaccaacaccgtttgtgttttgatttccgtgattggttatcatatatattctagtgtacgttgtggctttctccccggtctatgtataatatgaaccgtgaataatattccatttctgtaattatagtgggaggaaatggaattatgaaacttgttaatatctaattttttaaattttgtgtttttgttctggtgaattcaagacaatcatgaacatattgattggtagttaatttgcaatgaatcttaattttatgtgtatgtttgattttgttttctaggttgacaagtttaaactcgagctcggttaaaacaagaaactctaaacgtgttaagtggttgtgttcagtttacttgtattataacacgaataaacacatcggtcatgttcggttcaacaattcaacatgccaacctaataccattgacacctatgtctaaatcgtctgaaagtttatgttatcatcgtttgaagatatatgatgttactcgaatg

SEQ ID NO: 65 - DNA, 1244 bp, Artificial Sequence
  Synthetic Construct
gtcacttagaagccgtatccaaattataatattgattgattatataccaaaattaaccgacacctttcaagaatcttctcgcttacttaatcgcgtcgggtacgtgtcgcatggtcctataaatacaaccctcacacaacttcttttatcacacaatcgattctctctaattcaatcaaaaaacaaacaaaccttcatttcatatttgatcactctttgttttatatttcaactttgctttaatcatgtctaacgaagcagtttaccatgatgcccctaaaaccgaagagccatgcgaaaccatcaccaccacactgaaggtagaggagcccgagaccgtggagcatgcggatcgtggattgttcggtttcgggaagaaggaggaggagaagaagtgcgaagatgctgcgatttcgtccgagtttgaacagaaagttcatgtggctgaacccgaacccgaactgaaatccgaggagaagaaggaaagtttgctagacaaacttcaccgatctaacagcagttccagcagttctagcgatgaggagtgcgtggatggagagaagaagaagaaaaagaagtcgttgaaggataaggtgaaggacaagatcgaagggcacaaagaaaagaaggaagaggaaaaagttgagaagtatgaggccgacacaagtgttccaattgagaaatatgaagaagtagcggtcccaaccccgactcccgccccagaggagaagaaagggtttatggaaaaaattaaggagaaactacctggcggacataagaaggcagaggaggagcatgttatagctcctcctccgccaccgccagctgtggtggcacatgatgaagaaggcgaacagaaggagaaaaagggtatatttgagaagattaaggagaaaatcccagggtatcattccaagagtgaagaggagaaagaaaaagagaaagtaaaggagtgtgattgatatgattcaaagccaaaagcataatggtttgtgaggtggcttggtttgctttgattacccttacaagttacaactataatgggtgggccttggatgtgtttttgcttttatgtcttttgaataagtgttttttcatactatttgtttgaactttgaaagttcttgtgtggtgttattttgatttgggttgtaagtctatacctttttatttgtatagaatgtattttgtttataaagcattatatattttaatgaaacttaattataaactaaaaaaaaaaa

SEQ ID NO: 66 - DNA, 560 bp, Artificial Sequence
  Synthetic Construct
ttgtttttttttttttaaagatatacaccccatgtagatttataatttcatgcgtaaaggacccctgtatttttttaaaatttcataaacaacaaccaggcagataaataaataaaatataaaaaaagaagccaaccaattaatagaaaaaatgtaaaacttgataagacccacaaactccatataaacaaggcctcagtccccgccatccaaaaaaaaaaaaaaaaaaaaactagaaaacacccccacacctcaaacaacacttaaaacaagcagtctgtttagattaactaaaagcttttcattaacaataatactacaaacatacataaataaaattatcagccccctaacaaacaaaataaataaaatataagaataaaaataagagttcaaaataaatgttaacagcaaaatcatgccaactaaaaaaaaaagcataaagaaaataaataatagaggccttaaaactattgaaaacctaagagctgccatagcagtcattaatgttataatacagtaaataatttaaagcaaaatattaattttggaaattaaaa

SEQ ID NO: 67 - DNA, 1486 bp, Artificial Sequence
  Synthetic Construct
ttttattttcttatccaaaaattcaaccttataaaatatgccgtacaaaacaatacaaaattaaattaacatattaataaattttgaatacaaccaaatattattaaaatatccattacaaaaaaatcaaagtgaatggtggacattggtggatgagcatcaaacctcaacgagtggttcgcaatggttcctcattggatttcaactcctcatcccacctccatttcaaccccattatctcctcctctcctaccaccatcatcaccaccgttgtttcaacttctccggccaccatggcagcctttacaaacctcatggtcaaacctcctacaaacatatgctccaccaacaaactccatgttttctcttctcattacaaccctaaactccaatctttcttcccaaccaaaactaaaactcactcaccaccactttttgtaacgaaagcctccgccggtgctgccacaaaaaccgacagcagcggcggagcggctgccaaaaccaacaaggaggaagacaaggtacaacacatccacacagtggcggagttcgaggctgcgttacgcgacgccaaacaaaagctagtggtggtcgaatttgccgcccgaaacagcaaacaaagcagcgagatataccccttcatggtgaacctaagcaggacatgcagcgacgtcgtattcttactggtattgggtgatgagtccgatgagatgacggaattttttaaaagagaaaagatagaaaaagtgcctcattttaccttctataaagggatggaaaagatccatgaagaagaaggaatcgggccggatcggttgatgggtgatgttttgtattatggtgatagtcactccggcgttgtacaactacataacagagaagacgttgagaagctaatcgctgatcacaaggaggacaataaactcattgttctcgatgttggactcaaacattgtggtccttgtgtgaaggtatacccaacggtgctaaaactgtcaaaaaaaaatggatacagtggtttttgcaaggatgaatggtgacgagaacgatagttgtatgcaatatttgaaagatatggatgttgttgaggtcccgacctttttgttcataagagatggtgagatatgtgggaggtatgttgggtcgggtaagggtgagcttattggggaaattcttagatatcaaggagttcgggtcacatattaactagttccgtagattaaaaaggaaagtcgagtattgaatattgattaaacatgttttagtaatatttgtatcgtgtaacattttatgttactatgtatttttgaacgttatgaatcttggttatgcatttacctaggataatatttaaggttgagaaccaaacatatgtagaaaaccaagataaacgctacaatcattatgctcaatttaatagaaataaaaaagaaaagagagtggggaaaattttccactctcttcatataacttatgctaaaaaaaa

SEQ ID NO: 68 - DNA, 614 bp, Artificial Sequence
  Synthetic Construct
ttttagtcaaaaattggtttcataccattgttgattattttctcataattgagtagctctcttacaaggaaaaggaacatgtagacatgggttgactattaaacattcaatttaagaattaatatttatgaaacatcaagtctcaacttccaaccatttcactgaaagattacaagttataataactataatctgtcacaaaactttaaagtcataaaacaaatactaccctaagtcccctcatttcaccctagattccaaacctcaccatccttcattgttttcttcgcagtcgaacataaataacttatttcgggttcatttttgaaccaaaatatgtattcattttcgttatttatatctatatatgtgtatatatataaatcaaaatcctttatgccatagccatcccggcatgaaactgcatagggctcgagcatagagagtcgacatattcatgttgtttccttgcgcctgatgcatacttttctttgtgttcatcgatggaaatctgcttgtaccggcattcttcactacaaaatgccttctcacctctgtacatgaaaatatccaatccatgaagctttttcttgcaaagataacatgaactcaag

SEQ ID NO: 69 - DNA, 408 bp, Artificial Sequence
  Synthetic Construct
taactcgactgtccaaaactgaccaaatgatttagtttacattcctccttatataaacaaaggaataaaataacatagataaatggccagttcagttaaaaatttcggacaatgaaatgagctgttaaacccttttcccgaaccatttaaaccttaaaatgcccgaactgcttcagttcagtcgttgaaacggtatataaacaaccatgacaaaccacccctaagaggaacacaaccagaatgatacaattgagttccagttgcatactgaaacattaatatttctttcatcattctccagcatgtatgtttgttggtaatatgaactaacatactataaacaaaatttaatatacacatcaatatgctgcagatgtaatttcaatggaccttatttggacatcattg

SEQ ID NO: 70 - DNA, 957 bp, Artificial Sequence
  Synthetic Construct
atcaagtgggtcaaagtcaagcctatcctcatgatcaggatcaatggtctgaataaaaagcttccactcaggatagtttccagcagaaatcgaatcataaagatcttgtgtagcatggctgtgattagctccaccaatcttaatggcttcatcttccaacaaacttttcactccacatgtcggtttccagtgaaacttcacatagtatgctttcccagctttgttaatcaaagtgtaggtgttaacaccagatccttccatgtgcctgtaatcttgtggcacaccaatatcatcttgaagaaatgtgaacatgtgcagactctcgggatggtgggagaaaaagtccaaaattctccagtcttcttgaatgtgtgatttagggtttggtttcaatgaatgaaccatgtccggaaatttcatcccatcacggataaaaaacactgggaaattgtttcccaccaaatcaaagtttccctctcgggtatagaatttcactgcaaaacctctagggtccctgagggtttcagggctaccacgttcatggataacagtagagaaacgaacgataacaggagtctggacaccaggggcacggagaaaatcagcacatgtgagggcagaaatgtcatgtgtgacctcaaaaaatcccttggcactagcacccctagcatgaacaacacgttcagggatacgctcacggtcgaagttagctagcttctccaccaaatggtaatcttctagaaggatcgggcctctggttccaactgtcaatgagttgttgttgttgtaaactggagcaccagaattggtcgtccaaaacggggcattgtaagcacttgaagaacggtacttgtaaggatccatggatatgaagggcgatgaaagctgtgatgatttgttgtggagaagactatttttgctctatacggaatgctggttttattaacaatgattgta

SEQ ID NO: 71 - DNA, 1991 bp, Artificial Sequence
  Synthetic Construct
ttttttttaaccaaccatttctatatttactccaagatattttacatgaaaataagtgtgttttactttatttatattcaatcacactatatacatatatgcacatgtacatcatgcgcccgtgagtgcaaaaatatctattgcatctactgttcacaataattttggaccactatttttcacaacttgttaaggtcactttccctaacaaagagcccatggaaaccatatggtacccggtggggcagcttcactgcagctacgatctcaagtgtcggcgactttgcgtccatcaccacaaatcttgactcaccagtgttctcgttatgcacatacgaaaccacatacccatcatcctcatccgcatcagggttgtctggttccctagccacaaagaatggttcaccaccgaaacaaccaggcccaaatatccggctagcaactatgcattcgcgacgatccacttcggatagtgacgcatctagcttaaccacaccagagatcttaggcatcggatcaccaactccacaatacatatacctgttttttacggcaacaaaccccggattaataacgccaaagtcaagattacgggtcgaaagtgggtgacgggaaaccataccggtttttaaattaatcttaactttctcaaccgatgcatgaattaaatccattctttctaacgtatgttcaactgataatatgttgggtgccaccattaccaccgtatctcccccatcctcttcccatgcattgatgcaatggatcacattaaaccccggaacctcaaaccacttcatttcggactcgtctttcgcgtaccgagggatcaacccgagccgtgggacctttccggggtccgcactgaccggggaccccccaccgagcatctccattgggctcatcccgatttggatctcggggaaaatggcgtagtttttggtgatcgcgaagtcgtggaggaatgaagggcttgtcattgagaagatcgggacgtcggcttgtttttctccgttttcgttgaaacggaaaaaagttaggaaaggggggactgggccgtaacgaaaggcgaaagcttctttcgttacggggtcgatttttgggtgagcagtcatgctcatgaataacttgccgtcaaagtcttcgcgtccgacggtgattatgtcaccgtcgggggcgagtttaacggcatatgggagatctgactctcccagtgcatatagtttgttgccaaaaaaggctagactggtattggctagaccaataccttttgtggggtcaaactgtccggccaaaaaccggccggcagtgactgccatgcgagctgcagaggcagtcacaccattaaaccccgaaaacacgtttggaataatggggaatcccgcttctttctctatcgaatatttgtatgttcttatatatcggctacataatgaagcttttccgttggaaatacgaatagcgtggagcatgccatcgccgtcgaagagatggtagggtcctcgcgggaggaactgcgggttcggaccattacggaaatacgccccgtcaaggcaacttggcaacgtgccctcgatgacctcacagtcagtcgggggcaattcatccaccggtgagaagttatccgacaacacatgtttcggatcaactgaaaccctcgctggtggatcaataaaattattaatgatgctatcaaaagcattgaaaatggtggcgggtaaagactgatccgccaccacaactgaactttcacgttttctgatatttaatgaagttggttgctttttcgccccactaggtcttttgtcagtggtagtagtagtggtagtggtgacagtttgcggtttttcttcaattctgactgaaaagactctgaatgcaggtggtggtgatgacgatgacgatggtggtgggtgggaaacggttggttttgattgggggaaaattgaaatgaaagatgaggaaagagagtccatagtgtggtaccggat

SEQ ID NO: 72 - DNA, 2109 bp, Artificial Sequence
  Synthetic Construct
cttagaccaaaaaaaaacatagaagaagaagaaacatctgagcttcctcctctgcttccatagcgtgtgcttcaacatcctataaataatacttcacaataactcatcatattcatccaaccgatcaaaccaaatcaaataaccacttattttcataacatattgcaggtatcatgtgtaaagttacaaagggtaaatctaatcctactatggatggacagaagttccaacaaaagtatgtgacagtgttcaaaccatgttcaaatttactagacttggcagctgcggatgatgtgaccgggtttattaacatggttgaacagaaaggtgtgagcgttgatgacgtcagcttatggtacggaagatgtattgggggtaggggtaatcaacaaaagatggggttcacagaacgaaccccactcatgattgcttccatgtacggaagcattcatgttttgaaatatattatagatacaaagaaagtggatgttaacaagtcctcctctattgatggcgctacagcgctccactgcgctgcatcgggtgggtcccctatgtcagtcgcagttgccaggcttttaattgaggcttttgctgacgttgacttaactgaccataatgggaaccgagccggcgatttaattgcccgtggcattaaaccgtcggctcggagaggtttagagatgttgttgaaggggttttcgattatagatgatagtgatgaagagcgagctgaaggaaaagaaattgttgcagccagaaaagaatacccggttgatgcaacgatgccggatattaataatggcgtgtacggttcggatgagtttaggatgtatacatttaaggtgcaaccgtgttcacgagcttacacgcatgattggactgagtgtccgtttgttcaccccggcgagaacgctcgccggcgtgatcttagaaagttcaactacagttgcgttccttgcccggagttccgtaaaggaagctgcgaaaaaggagacgcgtgtgaatatgcacacggggtttttgaatcttggcttcacccggcccaatacaaaactcgtctatgtaaagacgaaactttgtgtgcgagaaaagtatgttttttcgcacacagagtggaagagctacgaccggtgtatgcttccaccggctcagctctcccatccccaaaatccggatccgtagttacttcaatcgaaatgggttcattaagcccgctagcgctagggtcaaaccctaagatgtctccaccaaactccccgatgtggcagaacaaggcaaatcatttgacaccacccactctgcaacttgcggggagtcggttgaagacatctttcaatgctagagatttagagcttggaccggagagtgtaagaacgccacaccaacaacgtcaaacaatcgataatttatcgaataatctttacaactataccaaccgattcggagaattaaaaccgaagaatctagaagacattttcggatcaccagatcaatcgttattatctcaattccgacaaaattcaagccctcttcgcgcatcctacccgtccccaacctactcatcttccccaatgagtaaactgagctacgggttcgattcatcatccgcagtcgcacaggcggtgatgaattcaagaacaagttcattaaaacataggagccagagcttcattgaccgaaccggaggctctatgaggctcccgcaacaatctcccacttattcggagtggggatcgccggacgggaaattggattggggattcaacaatgaagatgcaaacaagcttaggaaatctgcatcttttggatacggtggtggtgcacttgctaatgtggatcatgaacctgatgtttcatgggttaatacattggttaaagatgtggggtttgagcagatggtagcataaaagctaaaaaaaaccttccatttgctctttctaatgttgtagcaatgttagatattgtagaagattgaaaatagtttcttcgttaggaaggtattcttttaattttttttgtttgatattatgttttatgtaattcatctgttatttttcccaaactggggatttaataatattgtttttgctgattaaaaaaaaaaaa

SEQ ID NO: 73 - DNA, 1327 bp, Artificial Sequence
  Synthetic Construct
gtttgaagccctgaatccctcactcccttaaaccaaaaaccttatcctctctatccccttccaaacccctaatccttaacaccaccaacaccaatggcttcttcaacatcatcccttcagctcctaaccctatcttcttccaaaaccctatcatccttctctaaaaccctaacacccacttccctttacttctcccctgcttcccttaaacccttccctaagctttttacttccctaaccaacaacagcaacagcaaggtctttgaaccacgtactgttgctgttgctgtgtcttctgggttggagcttgatgaagagttgtctagtgatggagatgaggaacagcaggagcaggagcaggaacagagcttttcccctgacctaaagatttttgtggggaacttgccctttagtgttgatagtgctgctcttgctggcttgtttgaatctgctggaaatgttgagatggttgaggttatatatgacaaagttagtggaagaagcaggggttttggctttgttacaatgtcatccgtcgaggaagtcgaggatgctgttagtaaattcaacggctatgagcttgatgggagagcattgagggtgaatgcaggtcctccaccgccaaggagggaggattcatccttcggtgggcagagagaaggcggccgtggtggcggtgggagaagttttgacaacactaacagggtttatgtcggaaacctagcttggagcgttgataatcttgctcttgaaactttgtttagagagcaaggaaatgttttggacgctaaggtggtttatgacagggagagtggtaggtcaaggggattcgggtttgtgacttacagttccgctgatgaggtcaacagtgccatcgactcattaaacggcgttgatcttgatggtagaaatatccgagttagcgttgctgaagccccacaaaggcgtccgtattaaaatatccaaagacggggtgcagcatgttacaatttaaagaggaaaatgatgcttattgtgaagctgcgactacctgcattttttttatgtaacttaaggattagcaggtttcataagtgtcattaagttttgtaatgaatgcttttaaactatatactgatttgtaatcaatcattttggtgtatgcaaattgcattcggttgaatcttctgtcatatgaatgttatggttattgtgaatttgagcctgcaaattatttgcataaaatttgtattgcttaatcaatcattttatttccgggttgagatgattgaacttcaaatgtctgaaaggattcatgttcctttggtcgcgtgcgaagctgtttcgg

SEQ ID NO: 74 - DNA, 990 bp, Artificial Sequence
  Synthetic Construct
cacatagtttttgttttttaacgaatggagacaaatggttgtgtttgaaggatgagtgaaaaaaatatgtgacaacgtttgtgagtgtttaaaaccagatgcattagagatgctctaacaagtagttgcatttcccatctaaatcaagtcaattatggtttttatttttccaagattttgacaaaatttggcataaacaaaatcaacttaacttgacacttcatataagatcttcaattattgataagatggtatacaacataaagtagtgaaagtcctcaataaagatgacttcattgctatatatgatatgaaaacatcattacttgatgcaaaacacatactcaaacctcctggactttggtttccttagccacatccacttgtgcaacaccaacatccttcttcacatcctcaaccacttgtgctgcagcctccttctttccctcatcctcaaccactagtttaccatccttagccaagtcaacatctttcttaacatcctcaacttgaaaagccgaaaccggaaaggttcggtaaaggccagaagtggctgtgaaagtgatctttccagttggtgggtcatcaactgtgatctcactcaatgtgatccacatcaaaagctccttacttttaacacctgtgagtttcttgatcttgaacttctcaacgtatgcagttacctcagtcgcataggatacgagcttactgatcttttcgaacttgtgtacctttgatttcttttgcttaagccacacaaagcctgtgtccttgacataaccacattcctcaatgtcctctaatggcaaaagaccattgggtaggcccatttctgttagcaaaaacattgacttttctttaccaattgcatccccatggtaaacttctgatgcacttgccctgatctcatctgtcacaagagacattttggtttggtttgtttttgtggatgaatgagtgatggtatgatcagggcaagtgcatcagaag

SEQ ID NO: 75 - DNA, 807 bp, Artificial Sequence
  Synthetic Construct
tattattattattattattatttaaactctgtaaccctacaaaccctaacctagataagctctcttctataaaacctctacggcaaccactatacgctaacgaacactccttaccgtaacccgccgtcaccatacggttagagatctgatacagatcgttaaccgtagctgacgataatccatcaaccgttatattataatccgtcaccggatacgctacaggaataatatccacgtcatccgccacgtcatcaaactcagatccttcatcggaatctgaaccaacggcggcgcgtgaacacaaccgcttaaactcttcctctaaccgaaacatacactgctgaagcaaatcctctgctttatccaagcatgacgtcaccgttgcgtcatcggacatgacactgccttcatggattaacgatatcaattcatcaacagcggttaggaagagagaagcgtctgaggggtttgaccagatgggttggtcaacggttaagtatttggagacacgacggtcaacggtttttaacgtctgttcgagtgatgatttagtgtcgtgaacggaaaggttaccggagagtttatcacggaatcggttatcgaaattggagaagatttggaggatgtcgtcggttaaggcggtatcggtgtggcttagggatttggctatgtgttgagcgacggctatgagcttgtcttcggtgttttcagacatggttatggaagagatgaatcttgaggattgaggattgaggattgagaattgagaatgagaataataatattattattattgctgttattgtt

SEQ ID NO: 76 - DNA, 698 bp, Artificial Sequence
  Synthetic Construct
attaaccctgatacaaaaggtatggcaagataggtaaactttttttttttttttcttcactcattagcgttatcacaaaacaaaagcctttgaaaacagaaagcaagcaaaacgacatcgtaagatgggagttgagaaggaagtgctccgtgccggaaacgggccgaagcccgccccgggtcaaaacgtcaccgttcactgtactggttacgggaaaaatggtgatctgaacgaaaaattctggagcacgaaagatccagggcagcagcccttcaccttcaaaattggccaaggccaagttatcaaaggttgggatgagggtgttttgggaatgcaattgggagaagttgctcgactaaagtgcacccctgactatgcttacggtgctggcggttttcctgcgtggggcatccagcctaactcaactttggtttttgagattgaagttcttcgtgctaagtaagtaacttggcactaaacagcaaatggcgtacgggtttttgatttggttactgcacttgctattataaacttccgaaaccagtcaagtcaaacttgtgtttttaatttttaatgcttgttttagagcttttactgtgtgggtgcctgttagtttgagattgtactgttatgcatgactttctaaacccaacttatttgtgaaaaagaataagagcatcattcgtgaccaaaaaaaa

SEQ ID NO: 77 - DNA, 1082 bp, Artificial Sequence
  Synthetic Construct
tttttttttttaactaaaatcggccgctctttatttcaattaaacccgttcataaaacagtatcaataccacagactctcatttgtcaaaccacctacctcttcatgtgggtaataatctgcctgaaaaggctacagtaactcataaaaaatggacactcacaagtcgcaaccacctacctgcttggttttctagaccaatggaagctcgccgcactcactgataacaactctctttgttgggcggtcacctctgtctgtgacttgggactcgatcagcttaactacatccatgccctcaagaacttgcccaaagaccacatgcctttgatccaaccatggagtctttactgtgcatataaagaactgacttccattagtattgggccctgcatttgccatgcttacaattccaggtccagtgtgagtcagattgaagttttcgtccttgaatgtccgtccatatatgctcttgccaccagtgccgtttcccttgtcaaagtcccctccttgaatcatgaaatccttaataacacgatgaaacgcagaacccttgtagccaaaacctttctctcctgtacagagagcacggaaattttcagcagtttggggcacatcatcaccaaacaacccaatcacaattcgccctacgctgttaccaacagggtttccaatgcttatgtcaaagtaaactttgttggttactttagcctgtgtagctacctcctgagcactagccttaacggagaagcaccgggacttgtgctgaacactagggtttgttgaagactgcaaccgcaacgagccagataaaaagctcgatgatgctgataaggttgacttagcaagcttgtttgatctgaaacacgagctttgtacggttttgaccggcgataaagtacgggaagcagagagggaaccgacgtttgacatagatgtgaatgagagcgccattgatgagtggtttgggttggtaggataatagagagaggggtgtgttttgagtgaggggtaggggaaatggatgacgatgatgacgagtgagtggctccttggtatattcgctctttatttacagcaatg

SEQ ID NO: 78 - DNA, 2139 bp, Artificial Sequence
  Synthetic Construct
tttttttgttgttctgcaaataatatgccacttatttaattagaagtattagaacttattacattgttagtgttatctaagctcattccgggttgtgatgttacacaaaataagagaattttcactcttaagacgatcgaacatgcataacaattctcggtacataacatgtaccttggaattcttttcatgcttcaaatacataatacgaccgaccttacctgctttctaaattctaggcagatacaaggaagaaccaaacagaaacacagacaattcacaatcacataaaaacacaaattaagtctctcaacaacaaactaaaaatgaaacgacattgattcacacgttgtgttcctcatttattcaatagcggatttcattctaaatatttacttcatttaggcatcggcggtctcactagaatcagcagctgcaaggatcagctcagggtgaacacttcccacacttgttccgtccaatgcattcggcagcccaagtgccttcaatgccaaatgagccgctttctgtccggatatcatcatagccccaaatgttggccccattctgggagatccatcaatctcagcaacttccattccagtaacaatcataccagggacaatctctctggtaagcttcacgatggcgtcctcagcagtgttcatgtcgagcgccttcatcccaggaacgctttcgatcattccaacgctcctgagccgcttgacccccgtagcacccatgggaccatcatgaccacaagagctcacaaccaccttagcctccatcacattagggtccatgcatgactgtgtgtcatggttcatggacaccaaagcccagttggtcaccacaccaccaactcttccgccctttatgatcaagtcctctgctgcaaccgcgttgaagagcttcacatttggtctagccaaaagcttgctcatgatggtggaggtgaacagggcagcgtgcttgatgacaacgtagtcctcttgctcgtcgtactcaatctcaagctcgtcgaggaagtggtgcgctggtttgcggacaaccatggctgaaaacagctgtcctccgagccatgctccaccgccagggctcacagactgctcgatgatggctacctgaacgttagggtttttgctaagttcataggcacacgagaggccagcagagccagcaccaacgacaacaacgtcggtatcggcgtaagtaatcatatcagtcatgtaccgtctggtcatttcacgtgacacgatggattccttgatgggttgaaaggtgaacgacccgagatcgtatggagttgcggacatggagatcggtgtgtttttgttgttgtgggtcgatttaacgggttggattcggtttggagaagagattggaacgccatggaaggatgattgttcgaagaaagaggttttgatgttggtggagagagatgaggcaaaggttgtagccatggaagccatattatgatcacttataaaaagcttaacctataaaagcttgaaggtggtggttgtggtttgtgaagaaggtgagtgaggtgtgtggagttatatagcggtaagaggcgaaggaaatatctggagggggcaaagtagcccgaagcttttcttacgggtagggtgtgcaatggttcagaaccggactgaatcaaaccgatttaattcgaaaccgattctgaactgttagatgctaagtataaagatcggaattggactgtatgaaaacctactggctcagtttagaatcgagttaaacagtttatttagcatcttatcgaaaccggttctcaattaaattctcaaattaacaagtaaataaaactaaatggtgtgggtactaactcggttttagcagttttctgaacagaccagtggttccagactgaaccaaatcggaaaccgttttaaccaaaatttaagaatgaaaaccggaccctaatacaagaatacggtttttacctagaggttcggttccactgttctgacggtcgtggattaaagtcccgtttagagtaatttgtttcgggtttagtttgtttatttatggattgataggccgggttatcacgtttggttctttagacgaacgggtgaggtgactcattcgctagtttgatctctagtgtgc

SEQ ID NO: 79 - DNA, 986 bp, Artificial Sequence
  Synthetic Construct
aggacttgaagagacaactttttggtctgccaccaagatacagggactctgttcgagcgatcacacctggattacctctgtttctctataactataccactcaccagctgcatggtatttttgaggcaacaacttttggtggttccaacatcgatccaactgcttgggaggacaagaaatgcaaaggcgaatctaggtttcctgctcaggtgagaatccgcataaggaaagtctgcaaagcattggaggaagatgcttttaggccagttctgcatcactatgatggtcctaagtttcggcttgagctgtccatccctgagaccctggacttgctcgacctctgcgaacaaaatggggtttaaattatatgttgctgcaagtattacctcttttaagtgtatccgtatcgtgcataagcaaaagatggattagggtaatattgtgcaagtctagtaggtggttaagcaacgttttcttcatggagcttctgtataagcgtaagcttgagtgaagttgttttgtgggggtgccggactacaaacttggccggttgctgccgatgaataaatgggtcgatggtgtttgtgttacttttaatagctagctatagcttatgcaatgaacaaagtatgggataacctgtgtgtaatggcaaaagtcatcgtcggcctacatagatgacaaatatccttgaatgtttttttgttttgtggtgataaaattatgtttcattaggctggtcgtggttgtattgtgtttcgtattgtttggggaccacaccgatcatggaggtttggcaagttatccaaccacatgtccaatttattataaatcaacgatgccaaaccgaaagaaactggtttcatgttcacgcatgttggtgtggtccaagtttgtctcctatcttacagaacacacacagttacgtagcatccaagagtttccaatgaggtctccaaaaaaattgttcatcttttttttgcaaa

SEQ ID NO: 80 - DNA, 760 bp, Artificial Sequence
  Synthetic Construct
tggatgtatttcataaagaaacacatgctttcaacttctgatatgtataagtagagcaaaaaccactatgttaatattgttattacaaattaatttaatgtaagataaacaaactttttattattattacagcaaaatcaatctggaggttcagtttgcatttgctggggtaaacttgagggagacgctattgacacaatggcgttcatcggtgggggtacgaaaaccttcacctttaaaaacatgcccaagatgcccaccacaagctgcacatgtgatttccaccctcattccatctggatcagcatttcggtttatggctccagggagaccctcaaagaaggcaggccagccacacccagaattaaactttgccgtcgacttgtacagaggggtcccacatcctgcacaagcgtacactccctcctcatagaacttgtcatatagccccgtgccaggatactcggtgcctttctgtctgagaatacgaaactggtcaggagataaaatggcacgccactcctcttccgatttctgagccatagccacaactcttcctctgaaccctctcttagatcccaccaagacttgtgcaaaactcccaatgtttgttttcaattgcaaatgactgctgctcttcaacaataccctgcctggtagtaatgataatgatattgatactgacgtcatcgcaccacttcaatacctaacccctcacctcaccggtgggttacttaaaacaacgaacgaactacatgcc

SEQ ID NO: 81 - DNA, 937 bp, Artificial Sequence
  Synthetic Construct
aatcggcctgccaccgacagtacacacagcaagaggaatacactagactacacccaaccaaaccatatctcttcttcttatattattccccccttccactccttccccttctccttatcctttcccctctttactccaactcactgagtccacccgcttccccaccgagtcacacctttttccgccatgaaaggagccaaatccaaatctgaatccaaaaaaacccgacagcaggctttcagttaagaaaagagaagccccgaagcagaagaaagcgcctgcgaagaaagcgaaggaggtgaaggatcctaacaaaccgaaacggcctgccagtgctttctttgtcttcatggaggatttccggaaacagtacaaagaaaaacatccaaataataaatcagtttccgtcgtcggtaaagctggcggtgagaaatggaaatcgatgtctgaatctgagaaagctccgtttgtagctaaagcggagaagaggaagtcggaatatgataaaactcttcaggcttacaacaagaaattggctgagggaaagcatgaggaagaggaggaagaagaggagtctgataagtcgaaatcagaagttcatgacgatgaagacgatgatgatgatagcggagaggatgaggaggatgatgatgaatgaaaaagaagaaggaagacacgtggcaagttgttttgaataacccttgttttaatggaaggttaatttggatggtagtgattagaaatgcaaaagtagtttcagacctatagatggttgaaactcatatcctatctgtactttttttaaatccttaatttcaatcaatctgtgaaatatctatgtataacctaaagatgattcatagtgttgttggttgtcgaccaaaagtaacctttaatacaaactgttcgtggttgtttttgcttggctttaaaacaa

SEQ ID NO: 82 - DNA, 908 bp, Artificial Sequence
  Synthetic Construct
ggatatcaactgagcttttggaccagattcaacacacacacaatttacacacacactctctttgcaaaacaaaacaatggcctccttcccagttgtgaacatggagaagctcaatggagaagaaagaggtgaaacaatgaacctcattaatgatgcttgtgaaaactggggattctttgagattatgaaccatgggatatcaactgagcttttggacactgtggagaacatgacaaaagggcattacaagaagtgtatggagactaggttcaaagagatggtcgctagcaaaggcttagaagctgtccaaaatcaaattgatgatcttgattgggagagcactttctatatccgccatctccccgaatccaccatctccgaaatccccgatctcgaagacgaatataggaaggtgatgaaggaatttgctaaagagcttgagaaactggctgagaacattttggacatattttgtgagaatttggggctagagaagggttacttgaagaaggctttctatggtgctaagggtcctactttcgggactaaggtgagcaactatcctccatgccctaagcccgacctaatcaagggtttgagggcccacaccgatgcaggtggcgtcatcttgctctttcaggacgataaggtcagtggactccagctccttaaagacgacaagtggattgatgtcccaccgatgcaccattccattgtcattaatctaggtgaccagttggaggtgatcaccaatggaaggtacaagagtgtgatgcatagagtgattgctcagaccgatgggaaccgcatgtcgatagcatctttttacaaccccggaagtgacgctgtcatctatcctgcgccacaattgattaacaaggaagaagagaagaacaatgtgtacccga

SEQ ID NO: 83 - DNA, 2573 bp, Artificial Sequence
  Synthetic Construct
ttgggaacaaacaaacaaacatcctcacttcctgttatatttaaccaatttaaagattctatttttaaggaaacagggataatagacaacaacatgcatagtctagaattagaatacagataatttaatccaaacaaagtctagagctaaacataatagctcaattcatacaagtaaactgaatattaaaataaactgagattaaaaaaaactgaaatcaaactcagatttttcttctcaatgccattagattcaccaaactgccaactttttttagcacctcacacttttggagagatgattatgaaaccagaaccaatggtgtcgaatcaatggcgcgaatcggaggaaccagatgacggcttgtcagacaagccgtcgtgttcctcctcctcaccctggattcgtgccgggacaccaaaccccggaaaccctcccatgcccggattcccggaaaaaaatccgggcacggcacacgggtgataaaaacccggggtttgggtctgacccgggtcgatccccgctccaaacgagagcgggtcgacccaagcacgaaccggaggtgagttactggactgaaggggtcccctctgtgaaatatagtttaacaacaactgattgtttgttggttggccgaacaacggctgcagatacggcgtcgtttgtcccaccaccggcaccgaattaaacacatacccaccgttctccggccaacctaagccaccatcaaaactaatattattattattattatattaaataaataaattatctctctctctcttattctctcttttattcaacatgtttattgagagctttaaggttgagagccctaatgtgaaatacgcagaggatgaaatacactctctgtacaattacgacaccacggagcttgttcatgaagagaaaaacgggtcttatcaatgggttgttaagcccaaaaccgttaaatatgagtttaagacccaaacccatgttcctaaactaggggttatgcttgttggatggggaggcaacaatggctccaccctgaccgcgggtgttatcgctaatcgggagggaatctcttgggcaactaaagagaaggtgcaacaagccaactactttggttctctaactcaagcctcaaccattagagttgggtcatacaatggagaggagatctatgcccctttcaagagcctcctccctatggtgaaccctgatgacatagtgtttgggggatgggacataagcagtatgaacttggctgattccatggccagagctaaggtgttggacatcgacctacaaaaacagttgaggccttacatggaatccatggtcccactccctggtatcttcgaccccgacttcatcgctgcgaaccaaggcgagcgtgccgataatgtgatcaagggaaccaaaaatgaacaaatccaaaaggtcatcaaggacattagggagtttaaggaggctaacaaggtagacaaagtggtggttctatggaccgccaacacagaaagatacagcaatgtcatggttgggcttaatgatacaatggaaagtctttttgcatcattggacagaaacgaggctgagatttcgccatcgaccttgtttgctattgcttgtattcttgaaaatgtgcctttcatcaatggcagcccacagaacacttttgtaccaggggttattgatttggctatcaagagaaacagcttgattggtggtgatgactttaagagtggtcaaaccaagatgaagtctgttcttgttgacttccttgttggagctggcataaagccaacatccattgtgagctacaatcacttgggaaacaatgacggaatgaacctttccgctccacagacattcagatccaaggagatctccaagagtaatgttgttgatgacatggtttcgagcaatgccatcttatacgagcccggggagcatcccgaccatgtggtcgtcatcaagtatgttccatatgttggagatagcaagagagcaatggacgagtacacgtccgagatattcatgggcggtaaaagcacaatcgtcctacacaacacatgtgaggactctctgttggccgctccaatcatcttggacttggtcctacttgctgagctcagtacccgtatccaactcaaagctgaaaccgagggtaagttccattcattccatccggtggccacgattctgagttaccttacaaaggcccctctggtaccgccgggtacaccagtggtgaatgcactagcgaagcaaagggcgatgctggagaacataatgagggcatgtgttggattggcaccagaaaacaacatgatcttggagtacaagtgatgagggcatggcttatttattttataaatacgttatttgcttctttttttttcaaatgtaacgtgtgtgatgtgatgtgctatatgctttgctttgctttgtttcttcaagtattttatttggagatgaatatatcccaaaataaacccaagttttctggtgaatatttgttaatgccaatcatgccataaacacttatttaaaaaatagggggttgttgagactcgattagtttt

SEQ ID NO: 84 - DNA, 1619 bp, Artificial Sequence
  Synthetic Construct
atcctgaacttggtctggtttttctctttacatttttttttaataggtgactttctgtattgtggctgagtttatcacatccctcagaagtatctgggggtccctagccacacactcaacggcatcatcaggccaaagcctggtgttgcgggaatggtcggcccaggagaggcagtgcccctctgtgcaaagggcgctaaggctaccacccggaaaatttcctgccacttgacagatgttgcaccttccacaagaggcaagcctctctgaaataaatgcacggtactagaaaactagggggaccatcagaatcgaacctggcacctacaaatgatactttgtttgtctaaatttttggtaactcttaaattcaatccaaaagatttattaaatatagccagataaagtttttattttccaacaatcctaaattcttgaacaacacagagagaaaagctactatttttaaaccatacatagaccagatgagccaatgctacgagccttactcggaccggttagcatacctttcaacattcttgtcaaggttcaacccaaccatggcttcacctagcccgcagctcacctctgcaaggaccttagggtctgtgtaatgtgtaaccgcttggacaatagcacgagccctacgagccggatcgccactcttgaacacacctgaccccacaaacacaccatcacatcccagttgcatcatcagggctgcatcagctggtgtagcaacaccacctgctgcaaactgcaccaccggaagccgtcccaattgtttggtctgcatcaccaaatcatatggcgcagcaatattcttggcaaaagtgaacacctcgtcatcatccatgtttgcaaggaccctgatgtcacccataacagatctaacatggcgtacggcctcaattatattcccagtccctgcttcgcctttagtccgaatcatggccgccccttctctaatcctcctcaacgcctcacccaaattcctacacccacaaacaaacggaatcctaaaattatgcttattaatatgattcccatcatccgcaacagtcaaaacctcactctcatcaacataatcaaccccgatcgcctccaatatctgcgcctcaacgaaatgcccaatccgggctttcgccatgaccgggatggtgaccgcctgcttaatttcctttataagctgcgggtcgctcatccgggcaacgcccccctgtgcccggatgtcggcgggcactcgctcgagcgccatgacggcgcacgcgcccgcctcttcggcgatgcgcgcctgctccgcgttgacgacgtccatgatgacgccgccgcggagcatttgggctaggccaactttgacggaataagttgaggatttttttggtttcggttaaggcgccgttgccgtatactgctactactcctgtaccgtccattggtttggtttggtttggctaggttaaaccctgtgtgtgtttagggttttgtgtgtgtgtgtgtgatgataaggaggattggtgagtgaggcggttatgtatgatatggagaagagcaagtggtgtctgttttggtttatgtgatgggtttggatagttttggtcatttgacgaagaagcctg

SEQ ID NO: 85 - DNA, 842 bp, Artificial Sequence
  Synthetic Construct
gacattttcttaaaccgtcaactcgtttaacacatttaacacatttacaactataactacaactcattcattttcgacccaccaacatgttttgagtcatttaaacaatgaggtttctagattacacaattctaagtatgttcggggataaacttgatgtgtttgacacggataaatacatgggttgtttttggattcatcaattcaacctccaatgaggtcacaaaacaaaacaaagaatggctaaatcccaacagtttcattttcaatctgatataccatttccataactggacctagtacactttaggcgtcacattacatagaaatcaccaacaacttaaacacaaaacattaataatgcatattagctttcaaatctagtcttgatcaactcaagcggcggcagcatcggcttcaagcaattgtttcgccttggtgatgtagtcgttcattgcttcctccttggttttcgcttcaacagccttccatgcatcccatttagccctttctttcatgctgaatatgcccggacgatcagtgttcactggtccaactgtagcttgcttgaacaagccatagagtatgagcaaatccgcatcagatggctttttagtcaaagtcttgactttgtcagcatgctcttcaaattcctccttcaaacccatttggtgacgaaacaatagtgtgggtagtgacggtggcgatgtattcgtgactgacgagtgatgggttcttgttgctatatttatagccacaaagggatggatgttgatttatttgatttttgtttcggcggagatatcgagaaaacatgccttcaagttttataacctttacaa

SEQ ID NO: 86 - DNA, 542 bp, Artificial Sequence
  Synthetic Construct
tagtaccatattaatctcaaaaccttcgaatcaacaaacatgtcttgctcaagtggaaagtgcaactgtggctcaagctgctcatgtggcagcagctgcaactgcaactcctgcaacgtcgagatggcgaccaccaccaccaccatcatcgttgacggtgttgctcccaggatgacttttgctgagacaaccgagaccgctgagagcggaaacggatgcaagtgcggaggaagctgcaagtgcgatccttgcaactgctgaagatgagatatggtgccatgacccaaacaagaagtggtgttttgttttcatgtaatgaataaaggaaacctatatatgtgttatttgtatttctaatggggtttcattgtattgtgtaacagtcaaaacgttattttaatcgacttgtttgggtatttatataaagtttgtaattggatgtatgtgtgtgacgaaagaatgaatgaagaagctgtatgatatgtccaaagaaactaaatttttgttaaaaaaaaaaaaaaaaaaaaaaatttttttttttt

SEQ ID NO: 87 - DNA, 399 bp, Artificial Sequence
  Synthetic Construct
aatccggtttttagatcgagtgcaaagagtggtgcagagtaactctccttgttaccagcttgttgcccgtttacctaccgctacccaccgctaacttgtaattcttgttggtctttcttctaatattatttaagttaacgtaaaaaaaaaaataggattataatttcactagaaagatgtagaatcataagtcaaactaaaccaacataaccaacataaaatcaacaattcactaatctatatttacacataactataacaacatacaaacacataaccttcaaagcatcatagctttaaactcatcaaagttcaacacaccatcaccattaagatcaaactgcctaatcatcccaacacactcatccacactcctcgactcaccgagtcgactcagca

SEQ ID NO: 88 - DNA, 2756 bp, Artificial Sequence
  Synthetic Construct
aaaaaaaaaaaaaaaaaaaactcatccaaatctaaaatccaaaactaaataataatcaaacaaacttcatcaccaaaaacacaaatctctataaccataacccacatcacactcataaacataaaccctagatcaacaaccaatcaacaataaccaaccaaccaacaataaaccaaatgccaatgcctaaaagattcttccaagctcgctctcaatccagcagcatccccaacagatcaatcttactaaccctatgcataataatctccatttccggtctattattattcatcatctccctttcacgtggagagaccaaatgcaagcacaccaaacctgtttcagtctccgttgtttgggataaaactggcggcgagtctagtaaagcaagtgatggcgatcggaaacggcataaggttatgggatttgttggaattcagaccgggttcgcatcggttgggaggcggaagtcgttgcgcaagacttggtttccttctgatcatcaagggttgaaaagattggaagaggcaacaggcttagctttcaggtttatcatcggcaaaactggtgatgcgtcgaagatggcggaactgaggaaagaggtggatgaatatgacgacttcttgatgttggatattgttgaggagtacagtaagcttccatacaaaacgttagccttcttcaaagctgcgtatgcactctacgattctgagttttatgttaaagctgatgatgacatatatctaagaccagatcgtctctcgctgctcctggccaaagaacgttctcactctcagacttatcttggatgcatgaagaaaggtcctgtgttcactgatccaaaactcaaatggtatgaacctctagggtatatgcttgggaaagagtactttcttcatgcttacggtccaatatatgctctttctgcagacgttgttgcaagcttggttgctctaagaaacgacagtttccggatgttcagcaatgaggatgttactatcggtgcatggatgcttgcaatgaatgtcaaccatgaggacaatcgtcaactttgtcaacctgaatgtaccgctacatccgttgctgtgtgggacatccccaagtgttccgggctctgtaaccctgaaaagaagatgttggaacttcataaaatggaaagctgtgcaagcagcccaactttgccaaccgatgaagacgagtaacaggtgcacaaacttaatcttcggaaggataccctttcattcttaaattctacgacaaagccttagataaattttgtgtgtttcagtgtgtgtatgatgtatctcattctttcattatatatcaacttgaccaacaaaagttttgaaatgagagactgtaatgtaaaaactattttagaggttagaatatctagatgaaaatacttacctgctgcttcatgcagttcattgtatatcttgatggttttccttttaacataagttgtaatttcatttcttaaaacttgacaagttacattaaaatagaaggtagacgggcaacaatggcttgtgaagacggaaaagaaccgttactgaccgaacaacatgaggctcaggagcctaaacctcaggatgactgtatagtgtcaagcaaaagcaaaaaaactaagacgatgcagaagcgcgcctatggttgaaccgattattcaagaatccaatcttaaccagtttcttgaaagcccgaaatcgagctttgatcagtttaatccacagtttaaaagggtagctacttacttgtttatttaccttagcataagtacattctgtttttaccttgttagaaaccagatttctggacagaaaacaaatggggttctagactcgatttacttttgtattgtaaccatgacaacggttgggtatggcgatctaataccggatagtactgcaacgaaattacttgcttgtggttttgttttctcgggtatggtactcgttgggttctttttgagtcgcgctgcagacagtttggtcgaaaatcaagaagtgttactgatcaaagcgatgcatctgcgtcgagaggctaattcgatggaaacgcttaaggaaatggaatcaaatagagtgaggtacaggtgtataatattctccatcttgatggtgattctcatagcttctgggacagtgtttttaaccacagttgagaagatggagattgtcgacgccttctattgcgtttgttcgactatcacaactctaggctatggcgacaaaagcttctctactccggttgggcgtattttcgctatattttggatattaacaagcactatatgtttagcccagttgctgtttgcttttgctgagctatatacagaaaacaggcagagatcacttgttaaatgggttcttacaagaagaacaactgctgcagaccttgaagcagctgatctagatgatgacggagttgtgggggcagcagaattttgtatatataagctaaaggagatgggaaagatcaatcaagaagacatctctcttattatggcccagttcaatagccttgatgtggatgaatcagggactttgtcaccatctgacatttaccttgctcaatcaacagggtaggccaggtgaattaaacccagtaataatgacattcagatgtttgttgttacatatctaccatgtatcagatacatcatttcactgtacctcacttcatgaattattattaatatatacaatattaatgtgatcacaataaaccaatctcaaaatgttttaaaaaaa

SEQ ID NO: 89 - DNA, 2180 bp, Artificial Sequence
  Synthetic Construct
taataagccgtcttaaattcttcctacatcatgagtcacattattgccagttgctacaaacatatgctatttacgtttcttcataatgcctactgttaccagccacaaagattccggttttaatccacaggaggttttgtttttgcatttgatttgaatctgatttcatttgtctggcctgaatactagtggtgttgattgttgaaactgtttggagactgcaaggaccgttggacgtattgtttgtgttattatggccaatgaaaagaacatggagcaaacagaacacactgttgaagatgtaatggcagctccactaatagctgaagatgttaagagcaaagaggataatttcatggtttacataagtacatttgttgcagtttgtggttcttttgcatttggatcatgtgcgggttactcgtctcctacgcagtctgctattagagaagatttaaacctgtctcttgcagagtactcgttgtttgggtcgatactgacatttggggcaatgattggcgcggttgcaagtggacccatggccgatttcttcggccgcaaaggagcattgagaatatcgaccgctttttgtaccgcagggtggctagcaatctactttgctcaggggcctctgcctctagacattggaagactagcaactggattcggaatgggcgtcttttcctacgtggtacctgtgtttatagctgaaatagctccaaagaatttaagaggtgcattaacagctgcaaatcagctcatgatctgtacaggagtgtctgttgcctttatcataggaacggtgctgacgtggcgtgtcttggctttgacaggattgattccttgtgctgttctattagtcgggctgttttttgttccagaatctccgagatggctggctaagataggaaaacataaggagtttgaagctgccctaagaaaacttagagggaaggatgcagacgtgtctgcggaggcagatgaaatccaggattatatagacacactacaaaagctccctaaagccaaactattcgatttgtttcaaagaagatatctgcgatctgtcactattggcgttggactaatggtatgtcaacaatttggaggaatcaacggaatctgtttctacacgagcagtatttttgagtctgcaggattttctgctgatattggaacaataatctacgcaattcttcaggtcatcattactgccttaaatgcactatttgtagacaaagccggtcgaaagccactattactggtttcaggcaccggattggtcatcggttgtttacttgcagcggtttcattctacttgaagacttatgaaataggactagaggcagcacccgcgctagctgtaactggcatactgttgtatatagcttcattctctgcaggaatgggagcagttccatggattataatgtctgagatattccctattaatatcaaaggggctgctggaagcctagcaacacttgtaaactggtttggtgcatggcttatatcattcaccttcaattttctcttaagctggagttcttatggtacattcctagtatatgcggctataaacttggctggtattatgtttattatcgtgatggttcctgaaacaaaaggaagaactttggaacagatacaagcagccattaatgcttgatttatcacaacttatcgccatggtattgattgcagggtacatcaagaaagagagaaccattcagattaatgacgtcatcatgtgttgtacttaatttattttcttttttggtttgtaactcttatttattgttattgtcttttaactccatccatgtatcatttatagtaacttttagggtgcgtttggttcgcggaattgtttggaattggaatttgtgaaggaattggaattggaatgaattggatttggaattcaaacattccaaaaggaattggaaattgttggaattggattctcaaatcctatattattgtgtttggttggttaataaattggaatctatcacccttgcccccacgacccgcatcgttgtgacccgtaccggccccaaccgtttcgctccgtaccgacccataccatttcgaccagtgacggcccgtaccgtttcgaccagtaccgttttgacccgaactgttgatacctctatcgtttcgacctgtaccgtttc

SEQ ID NO: 90 - DNA, 822 bp, Artificial Sequence
  Synthetic Construct
agacctttatcgggcggcggaccctctgttgttgttgttgaccttgattcgatcttttcgatcacaatcgtgtgtgtgtactgatcactgactactgcaccatgagcttactcaacaagctctgggacgacaccatcgccggtccgccgcccgataaaggtctcggaaagctgcgaaaacagaatcttagtttccggtcattgaattccgaaaaggaatcatgtgaaacaggtggtgggatgaatacagcagcagcagcagcagcagcggcagagtatcccgcagcagcgaaagtaacacgaagtattatgattgtaaaacctgagaggactctaagcgacacgccgccggcttcacccgccggatctacgactccgctctctcctttttccggagaatcattcaggtttagaaggaagtcggcatcagatgctttcgagaaggcgagtggaattggaagtaggagccctcgtcctccttacgagctgtgaactttgaacgagataatcgtatgagtttatgtatgtgtgtttcgctttagtaggtgataatatcatatgtatgcatgctttagtacgtttgtctcttgagtcgcgactcctccttaagttgactttcttgtaagataagtgtcttaacatggttgatcttaatgctttgtgtttctggttccatcaaaagttaataatagcatatgatatatatatattaaattttgaatgtatttaattgtaattatctccccataattagtggagatattcctttgtatatataaggttacattatcaatgttatcaatcaagttgaaacaaaaaaaaa

SEQ ID NO: 91 - DNA, 766 bp, Artificial Sequence
  Synthetic Construct
gttcatacttgcttaattatattctcatctcatccatcccagccatacttgctaaataatattctcattcatccccggcccaaaatactactttaatttaagctttaagacaagaaacataaatatcatcacaagtaaaccaaaaatctagcttattgtcacaatacaagtagcaatcaaatcttcataatatgaaagattaacacaggaaaaatcaaacacactatcaaaaagaagagacttaatcaaagagagagaagcccatgtcttcatcatcactctccttatcttccttcttctcctcagctgcaggtgcctcagcagcagcggccgcaccgccagctggtgcagatgctgcagcagcaccacctccaaaggaagctccaccactagctccaccaataatcacctgaaacacagcagaatcaggaagaacatagctgaaagacgactggacaccaccagaagagtcacaggaaagagcagcgttaacaagcttcctctgaagagtgtatgtagagtcagcagagccttcaaggtctccgttaagctgcttggcggtccagtcaccgcctgaagatttgcatacgaaggtgaacactcccattattattgcttgtgatggtttgatgagatcacaggcaacggcggtgtgtgttttgtgagggggtttgtgttggtgatgtttttgctttatataagtgtgtggctggagttggagatatggggagccctaattgtatctaaatgacggttttacccatgg

SEQ ID NO: 92 - DNA, 411 bp, Artificial Sequence
  Synthetic Construct
atcacagccgttcctgcaaccgaggaagagatattgggcgcaaaatatatggtggtagctccgtcgatgctatcggcgacacagtaaacgttttccgggcaacgatatgatgattagaatgttgggtttatggttttagaggctggggttgaggctgtagttttcaagttgttgtatgtgtatatagagatgttgcttttgtgtaataaagtttgggtagacatgaatgtataatttatggttgttatgtatttgcctaaagcgtgtgggccgtgtagtgggcttcacggtgttattgcaaagtaggaaaccgatataagtgcgcattgaattgaggtgccaatttggctatttataatttgtgacaaaacagggtgttttaaccggtttagcgcactgtagcgcgcagat

SEQ ID NO: 93 - DNA, 2073 bp, Artificial Sequence
  Synthetic Construct
ttgagcttagcatgctaagctcaaacttaaactatcgtaaattaagcaaggcaggctcggttttacacaggcgatcaaatcattgcatttttattattcttgacaagaaaactgagtgtcataacacaaacagatagcagtaatctaagaaaccaagtaaagaaaaactagcatgaatactgtcggattacatttggcgataaaatagttcccatctcaaactggtacaaaaaatagaccctatttggtcaaatatcacctctacgcttattaatcataccataagaccttgtggatcagaagccgccggtgcctttttcttaggcttctcaaccacaatagcctgtgtggttagaaccattcccgcaaccgaggctgcattttgcagaccacatctggtcacctttgctggatcaatgactccagcttccaccaaattctcgtatacatctgtcatcgcgttgtaacccatctcccattcgctctccttcactttctccaccaccacttcaccttcaactccagcattttgagctatgagagatgccggtgccaccaacgccttttgaatgatgtctgcgcctaaccgttcatctgcatcctcgaacttttctttaatagatggaaccaatgttgatagatgaacgaacgcagcaccaccaccaggaacaataccttcctctatagcagcaaaagttgcgtttttagcatcttcaatacgtagcttgcggtcttctaattcggtttcggttgcagcaccaactttaattacagcaattccacctgatagctttgcaactctttccgctagtttctcagtgtcgtaaacagaatcggtctcgaataattcctttttgatctgggcaattctagattgaatctcgtcctttgttgctgcgtcggcaatgatggttgttgagtccttggtgattgtgatcttcctggcggtaccaagttgctccacggtggcgttttgaactagcagacccatatcgcttgctagatactcggatcctgtgacgatggcaatatcttgtaacaaagcctttcttctctcaccaaaaccaggtgctttaatggcagcaacattgagaataccgcgaagcttgttcacaacaagcgtagccaacgcctcaccggtaatatcttcagcaataatcaacaaaggagctctcaattgagtggtttgttccaatatagaaatgatatcctttattgaagatatcttctggtctgtaataagaaccctagcattctcgaactcaacgagtagtttctcggggttggtgacaaattgcggagagatgtatcctctatcaatctccattccttcttcaacatcaacagttgtctcaaaagaagatgatgactcgatagataagacaccgtcgggtccaactttgtcaatagcatcagcaatcatggttccaatgatatcatcgtttccagcagagattgaagcaatggctttaatatcttcacggcctttcacgggtcgtgcctttttctccagctcctcgattaaacccgctactgtcttatcaatacctttcttcaacgacactgggttagcaccagaggtgacactcaggagaccaagtttaataatctcacgggcaagaaccgatgctgtggttgtaccatcaccagctgagtcgttggttctgcttgccacctctctgataagagcagcaccagcattttccatagggtcaggtaattcaatcgctcttgcaatcgtaactccatcattaaccaccttcggcgaaccatattcatccaacacaacattcctccctcttgggccaagagtaaggccaacacaatcagcaagtttatcaatcccagcctgcatagcagctctggaccgttgaccaaactcaatatctttagccgtagcccggaccgcaaaccgccggttcgaccgtctcatcttttgaacagtctggaactgaaccacccttttattaccatcaaacacactctttttgtgtgaagagaggattgaaggagtggagattgcattggcagaggccattggaaatgatgataagaggttttgggtgttgtgga

SEQ ID NO: 94 - DNA, 2874 bp, Artificial Sequence
  Synthetic Construct
ctgcgattcagtgctatacccaagtaaattctcttaccaccttacacatacacacacatacatgtatatgtatccatacacactgacattagcctccgaagacatgatccatagtaaagaagtagacatggtacgaatgggattgagaaggagctcttcccctttttggggtgcattatagcacagtccgtgactacgtttttccgttggttgatgctgctgcacacttgttatatttgtttatatagtgttagttctataacgaatttcaagcgcctatgaattggttccaagaaagtggccgttcacgtcaggaatcaaggttgttgatgtagttgctattctttctttttttcgtctcgattgggttggtgttcagtgtaccgctccgtgggtacaagatcgaaaagaatgcattggatggatgcccgggcattgagaaggaaggacgctttcagaggcgaaaggccatggggagataccgtctgtgatccatggatctccgatcgggaaaccgtatccaagctccgtggctagtctgcgctctttggacttttcaaacttagcgaactgaaacatctgagtagctaaaggaagggaaatcaaccgagaccccgttagtagcggcgagcgagagcggattggggctttgaagaaaaacaaagacgaagcttcgttcctcagcaaagtgttcatttctttttcgccaggtttcattctctttcttggatgatggaaaaaccagcaagctacggcttgaaagcttaccttcttctcttctggagaaagggctttctttatagatgaagacgttgagtaaggggggcggagcttgaagagcgaagcgagctgcgctagcctattacgtttttcagcagcaagctacggtctaacacttgcccccggtggctaacctaggttggggcgaaaactccaaaactagggttccaaacctttctctaataatcaggtaagctttcaagccccttgactttctattagattagtttagtcaagggcgctgtgaactgtaattgtgaaaaggttggaagatctggccaaagaaggtgatagccctgtagattcgttcccatggttcgatccttcccagtaaaacgcggcgtgttcgaattctgatcgcttttacgcgagaaagggggaccaccctctaagcctaagtattcctcaatgaccgatagcgtacaagtaccgtgagggaaaggtgaaaagaaccctatttagggagtgcaatagagaacctgagatccgatgcgaacaatcagtcgaaggagcggagcttagagccttgactttctatattagtcaagcgcactcactctaacggcgtaccttttgcatgatgggtcagcgaggaaatgggaacagcggcttaagccattaggtgtaggcgctttccagaggtggaatcttctagttcttcctatttgacccgaaaccgatcgatctagccatgagcaggttgaagagagctctaacaggccttggaggaccgaacccacgtatgtggcaaaatacggggatgacttgtggctaggggtgaaaggccaaccaagatcggatatagctggttttccgcgaaatctatttcagtagagcgtatgatgtcgatggcccgaggtagagcactcaatgggctagggtggccccatttcgccttaccaaccccaaggaaactccgaatacaggcctagatcgtttgtacagacagactttttgggtgctaagatccaaagtcgagagggaaacagcccagatcgtacgctaaggtccctaagcaatcacttagtggaaaaggaagtgatcgagcgatgacaaccaggaggtgggcttggaagcagccatcctttgaagaaagcgtaatagctcactggtctagctccatggcaccgaaaatgtatcagggctcaagtgattcaccgaagcgacgagaccttgaaagcagctttttcaagtgtcagtagcggaacgttctgtcaatcggggaaggtttttggtgacaagacctggagatatcagaagtgagaatgctgacatgagtaacgagaaatcctgtgaaaaacacgatcgcctgccagtggaaggttttctgcgttcagtcaatctacgcagagtgaatcggtccctaaggaacccccgaaagggctgccgtccgatgggtacacgaaagtgacgaagttgctttgactacagaaccatgcctgtctgttggagcgaattggatgatcgggccgagggctgccccctcttcccctcactctcctttccctaatatgaaccttgagtcatcaaagcctttctgactcggcctggcccggtcgccctacgcgactggcgcttcaaaaggcgaaactctcgtcgtagtttggcgacctaaggggcggacaccttttgttatctaaaggcgaagactcggaagtgggcgaacactcggcccagcgggcgaacatgccgggctccggctccgcgcacctgctgacacctttcgaagcactttatagtgtgcgctttagttttcgaatgtctcgttcagcgtcacgagtctacaagcctttctcatttcagtgctcgccctttgaatcgggcctttagtcttttgattagagtaggggtcgcgagagagcagagcgtaccgccctgccatagtcgcgagtctgtttatagtcgcgactgttgtcatagtcaacaaggttgaaacttccaggaaaaaaacttcgaattgggagggcgatcctcccggtgaactgaccgtaccccaaaccgacacaggtgaacaagtagagtatactagggcgcttgagagaaccatgtcgaaggaactcggcaaaatgaccccgtaacttcgggagaaggggt

SEQ ID NO: 95 - DNA, 242 bp, Artificial Sequence
  Synthetic Construct
atgtatgtatgtatgtatgtatgcaggttggcaggttggttaattagttggagctttgttgtacattatagagattcaattgaacataaatcagtctgcatcttcataatccattttcttctttattgatgatttcttctttaatggtgatttattctgtcattcttctcataaagcaaattcaaatctttgaaaagtttcttcatcttctcagtacagaagtttcctaaccagaggttaga

SEQ ID NO: 96 - DNA, 928 bp, Artificial Sequence
  Synthetic Construct
tttttttttctttataattattttcggtgtttttatatatgtatataaattatggaaacttaacaagctaaactttactatcatttcaaaacgtaaaccatctaccaggctgggttggacgcgccaacctggccctttatgacattgtctataagcaaggtccgcccctatgacgaacatacaatagattaactatctaagactcgttcaatggcctcgacaaagaagtctttaggcatggtgccaactacagactcgcatttctcaccgtttttgaacagcacaacaatgggcacagccttgatgtcatattgctcagcgactcgaggctcctgctcaacattgactgcaacacactcgagccttccagcataatcagtcgcgatctcgtcgatcatttggtgtaccatctgacatggttcgcaccaactcgtgtagaactcaactagtaccggagtttcactttttaaaattgatgggttccatgattggggtgtaacttccaaaactgcagattcttgacgcatagagaggaccagcagacgagaccgattcttcgggacccgaaacgacaaatcataggatgttttaagttggtcttgagccatggatggtttgaactggggaatggaacaataacgacgagttacaagagagtaagtgcaagaagcagcagccattgaagaaaggtatgttttttttttttggtgaataaagaacagtcactagttgcagagaatgtagaattgtagatctaatgagtatgaagaggaatactataggttataaaagaattggggtcagcaaatatcagaacttgattgagatatgatggtgcacgttttctgttttcattgatatttaggtgtgtagtgattcaagtattttaacttattgtaaggcataagataacataaggatattattg

SEQ ID NO: 97 - DNA, 587 bp, Artificial Sequence
  Synthetic Construct
tttctcccgggagcttgcattccttccaaacgacgtcgtattaagaacataaaaaaggttctagaaccctcttatctatctactccgataataaacctctatccatactagggttttgttatcctcttcatcttcatctccatttcatacctaccaacacattatcttcatctccatctccattcgataatggcagctactcacatgaccaccgccaacgccacgtcatcacgtgtttttctcccgccgtttgaaggtctccggccgacaacaatcaaggtttcgtctgtgtcgtttagaacgggtcgtggtttgactctcgggtcgacccgtggacttgttgttaaagctgctactactgttgctccaaagtacactacattgaagccgttgggtgatagagtgttggtgaagattagtgctgcagaggagaagacttcaggtggtatattgttgccatcgacagcacagtcaaaacctcaaggtggcgaggttgttgctgttggagaaggcaggacggttgggcagcaacaggtggatgtaagtgtcaagaatggtacacaagttgtgtattcaaagtatgctgg

SEQ ID NO: 98 - DNA, 504 bp, Artificial Sequence
  Synthetic Construct
gggttcaccaggtggtggaggcggctcaacttctcccaccggtagtcccatgggttcacctggtggtggaggctcctcctctcctatgggtagtcctgcggcttcaccaaatggaggcatgggcacctcatctcccacaggtggtcccgccgcttcacctggtgcagccggggcttcacccacaggtaatccagcagatacaccaagtggagccgcgtcttctcccactggtaccaatgggaatcaaggtgcgtcggcagcaagaccaatagttagtgcaatcttgagtgtgccagttgccgtgataataatggtatttatgtattagagatggtcatagattgaatctctttgtttaagttgttatttttcacagaatttattgtatttacgtagttgtgtgttaataaatcgttggatttgttcacgaatcatgatatatattaaataatttggagttctcttttgtgatctccattgatgattgaatgcatataacccggc

SEQ ID NO: 99 - DNA, 759 bp, Artificial Sequence
  Synthetic Construct
aaaggtcctgtcaattccatgctctccatcaagtaagatctcgggctcaacaatcgggaccagaccattgtcctgagaaatagcagcgtaacgagccagaccccaagctgcttccttgactgcaagtgctgatggaccgttgggaatgctcacaacagtacgccatttggcgaaacgagccccctgctggtagtaagcagcagaacgcgaggcaagtccatcaagaccttggcaccatgactcatcatttgaaccagccaatgggactagacccttgtcaaccttgataccggggacaatgccttgttccaaaaggacatcgacgatcttcttgccatcggttgtggattggtacaaagtctcctcaaagaggatggcaccagagatgtagttgcctagacctggtgcactaaccaacagggtccggtatgcctgccggttagcttctgtgttctctagcccgattgaggctaaccgctttccacaagtcgcgtttgactcgtccatggctaggattcctcggcctggggaggccacggttttcgcggttttaacaagttcatcagcatacgaactagcacggacagtaagcgctgagggtgcagccgagttaaaccggaccgcagaaacggggacttgtcgaagagtctggcccttaacccaatcagacttatcaagaacagttgctgcagatggcttgaaaagagaagctgaggccatggttggatgcaataagctaaacttctatcctttagtagt

SEQ ID NO: 100 - DNA, 1457 bp, Artificial Sequence
  Synthetic Construct
gaacacgttatattaacaaacatacatgaacaagatcttgtttttttttttttcggttcgtttcccgccctacacagggcccttcggaatggcggagcatcaatggcttttggtatttttaatattaacacatttctaaaagttgcaaaccaacactcaaaaagttctaaataccaaaattatcatcaaccattgatttacaaatatatttcatatatgatatataatcattaacttgtatctacaaccaactaaatcatcatatatctcccctaaaaatcaaatttcaatccgacaacatatacaattgaacttgtttcataagtagtttaactggaatcaatcacttaagacagtactggtaccatatccgaacccgatatgtgacttccaccaaagaaattcgtcacaatcgagtcaatatcctcgagtccaactccgatgcatagagaattcgtcggtggcgactccacctcttcgctgcgcaccatcacatttggcccgtctctacacttccccatacatttgcagcccaccacagtcgcttctccgcccacagccgcctggaaattctccaacagcattgcagctccagatttcttgcattttccccccatacaaacctctatcttcttaccacaatcagcctcaactacattgctactaatcttgcaagtggccatctctacgacttgtttttcggctttttctagaatttggaccgatgacgttgcttcttgatgggtggtactgtttgaggttggcacggttttcagctggcgcatattcacgacgttatcacaatcagactctgaagaactttcggatgaagatgaatccttgcaatccgtcttcggcatcatcgccatccgggcagctttctttttggccttctcttctttcttcatcctcttatattccatcttctcgtcttttagcttctgcaattgtccaagtaatacctcggtcgcttccgagatcatcttccccttaacttgctgacccaacccatcaccatcaccatcaccatcataatccaaaccaaaccccatatcagtaaaagcattcaagttcttagacaaacccttcaacaccttcaacttcttcttcacctccttcttcatctccttcttcacatctctttccttcacttccatcctcaccccttcataatactttacatgactcttatcacaaaactcaccactacccacaaccatattccatctcttcaccttcgcaaaacaccgtaaccgacgggagcttgacacgtcagcaacggctggagagaaacactgcgactgacggaacacaactccggtgacatccattaatattattaatattaagaatgataatataataatttgaatatgttggattaatgggttttgtgatgaagaatagaatagaagcaatttgattgaagcggtggatttcgttttatacaaggaagg

SEQ ID NO: 101 - DNA, 667 bp, Artificial Sequence
  Synthetic Construct
actatatgatatgaaaacaacgtaataaacaaccaaacagtttaaaagttagaccaaacaaatatagcggaagtagttttaaaagttacaatccataaaccaaaagtaaatagttcaagaagtttgaaatagtgtttgacaagatgcatgaaccagatgagacacagtgcgacccccgtgcgcctaatccgtgctgctaccttagcgaccatttagagtttatgtagttcttagcagaactaacgttccatttcaccaggaccagcaggtggcatcactggcaacacgactcgttcgtttttgcttgctggatttccttcccatcaaatggttcccttgtcgacaatctcagcaatttgtccaaaagtaatcaacggacaccaacaagtcaagcaacctatggttccgaaaacaatatgaaagtccaaaactagagcaatctttgaaacagtcacaaagcccggtggaccattgtctaggggcagtcatggacagtggtgggatctcaatggcgctttgtggagccggttcggtggaagaatccatggttacactttcgttgagttgaggccggttgaaatgttgaggacaatttatttatagcgacagagaatgatgtgtaggggtgtggactgaggacagaatcgtcttacgaagttatagtttc

SEQ ID NO: 102 - DNA, 753 bp, Artificial Sequence
  Synthetic Construct
gagttgatcaagaatgccaagtacatcgccacaccaggcaagggtatattggcagctgacgagagcactggaaccattggaaaacgtctctcaagcatcaacgttgagaacatcgagtcaaaccgccaagcccttcgtgaactattgttcactgcaccaaatgcgctaccttacctctcgggtgtcattcttttcgaagaaaccctataccaaaaaaaccgctgatgggaagccttttgttgatgtcctcctagaggccaatgtccttccaggaattaaggttgataagggtgtggttgaccttgcgggcaccaatggtgagaccactacccaagggtttgatggtcttggtgcccgctgtgcagccttttacaaggctggtgcacggttcgccaagtggcgagctgtgcttaagattggagcgactgaaccctctgagctttctatccaacagaacgctcaggggttggctcgctacgcaatcatatgtcaagaaaatgggcttgtgcccattgttgagccggagatattgaccgatgggagtcatgatatcaagaaatgtgcttatgcatccgagatggtgttggctgcggtttacaaagcgctaaatgagcaacatgtgctccttgaaggaaccctcttgaaaccgaatatggtgacacccggatctgatagccctaaggtgacaccagaagagattgctgaatatacagtgacaacactacgtaggacggtgccagcagcggtaccagg

SEQ ID NO: 103 - DNA, 874 bp, Artificial Sequence
  Synthetic Construct
tagcttagattgtgaggaagaagtggatggccatggcgacttcatcatttgcaggcatttcagctcttcctctctcacaacctaagctatcagccagtcaccttccatctaaagttctacatacaggaagagttccttttcataaattttcaacaaccggaaatcctgctttttcaacaaaagaatgtcatggattgtcaaagaaaatgatatcgatagcccccagatgcagtaattcctcaagctctgatggagccacagaggagtcatctaataaaaccaaggttccgtttgggtatagtcgaaaagatgttattcttattggagttggccttacagttatcggcgttggtctaaaaagtggattagagttcctcggagttgatcctctacaggccgggaatgtggtccagttagtgttggtgttagggcttacaatcggatggatttcaacgtacctttttcgtgtctcaaataaggaaatgacatatgcccaacaattacgcgactatgaaatgaaagttatggagaaacggttggaggggctaagtgaagcggaactagaagcgctgcttgaacaagtcgaggaagaaaagcgtttggcaaacagggattagttaaacctgggttgcgttatccttgtccggtaccaccatagtttagtaaatgactattacacgaccaaagtttaaatgctgtataccacaccctgcctgcaactatcttcatgctgtaaatgattattgcaattttatttttgtataaaatgaatttagatacttttaaaaaaatagatttagacaaagaagttaagaaaagcattgctacatatattaatctatctaattatctatatcccaaagggaagtgtcgg

SEQ ID NO: 104 - DNA, 1267 bp, Artificial Sequence
  Synthetic Construct
ttcgtggtttcctagcagtcaccttcacaccccaccttccatttctcttcgcaggtctggatctcaccggagaagacgaaggccgagttgtggttcccctattcaacaccgtcgaggcatacaccggccagctgtgcaggttattgtttcttcattggcgccatccgtctatttgagcatacaagaacgttaaaaacttctatttctcaaaaacagtaactatgtctctcataccaccgttaccaagcttcacaaacgctaaccccgacaccgacgttgcccagggaggcaccggccagtcgggaaacacagcagatgttcagcaacaaacaacgaacacgggtccagccgtcaccccatcttcagggacaaggcagaccccttccttccagtttattaccgaaacgaatcaggtttccccagctgagaataccgcgccgatggttagtccggaggtatggtatactcagcagaaggaagccctgaaacgtgtatacctccaaaaactgagggaccaacaactcagagaggctgtccgcgcgctttcctacagcgagggaccgtccaacacagcaagaaacaacggcgggagcaaccatgaaactgaccccacatggagagaggactcaggcgagtcccaaagtcgtttcacgcgtgacataagccagcccttaagcaacatcgctgcgcaagggcactttatgactagtgatgacgacctaaccaaaccgtattatcctccatcgttcatggaaaagtcgaaattcaccccgcgcattgcgctggcacccctgccaccaaaaacaaaaatcccgagtacactgggtaaatttgatggcacaggagatccagacgatcacctgagaacattcgaggtcgcggggaaatgtgcgcaatggagtttaccagcatggtgtcacatgttcgcgcagacgctcacgggagctgcgcgagtatggtttgacagcctaccaaatgggaaaattgataacatcaaagaactgacagacaagtttgcagaccacttcagtcaaaacaaacgaatcgagcgcgactcaactgacatcctgcaagtcaagcgacgagacaacgaatcaatcgaagactacatcacccgtttcaatagggaagctctgcagatcgggcccatcggtttcgaacttatgaaagcagccttcactcagggagtgcgatgcgactccctcatccgcacactagcaggctgtaccggaatgcccaacacatttgttccctttatcatcggtta

SEQ ID NO: 105 - DNA, 1214 bp, Artificial Sequence
  Synthetic Construct
tcaatgcacgtggtacgacttacgagtgagagcaactaatacaaacttttccttatatagatgacactctttagacaagtatatgttacaacgatgaaatttaaatttgatgatgtgaagagagagttgtgagatggatgagataaaaaaaattagataactcgtcgcataatgcctaagactgtacgttgtggggtgacgcctcatgccacctcgctcaaacccaacgttatacgccaaaacgtggccgtgaaagatgggattgaaacacttcaatctccagcttgtttcagaacccgcgactttggaaacatgcacaacctataaagtctagtcgaggttgcttaattcaactttcatagacaagctagaatgaacgcttgagcatgattcatcggtcatgcttcaaatctatgttgatagttattgataaatttatacatacgaatcggtacattataaacagagaggtttaagttttattttaactggatcttacatcctccatggctgcaaccaaattatcaaacttagccccagtaacttcttttacaattttaccatgtttaaggatcttgaaggtagggaccacctttatgcctagctcctttgccaagggcttattttcttgattgcagtcaagctttaagaacacaacatcaagatacttctcagccaattcttgaaacttcggagccattatcttgcaaggaccacaccactgagtgtacatatcgagcacaacagtcttatcaccggcggcattaacgatgggccaaaacgtgtccttatcaacctcggtcacctgtccgacaactacagcaggacccgaggtctccaagctcgatctcaccgtcactagagacctcagctttttatcgttaacggtcttcactaatgtgaaagatcgttgaacttctccggcgatggccgcgtgcttagtcggcaacgagaaccgtgggccgactgaagaggcgggagaaattgactggcagatttgacgaatctgtaatgccattgtagtagtgaggaaaacggttattctgggaacatttgagtggttgggaaataggttgatggtgtgcaataatgtgagaaaaggactgaattgtccttgtgtttggttgatttgatttgatggaatatcttagggtagtttgggaaatggagttgttcacataacgaccaaattaaatggtggttggttgtgtggt

SEQ ID NO: 106 - DNA, 237 bp, Artificial Sequence
  Synthetic Construct
tcattgaacccattcttcccttcaccatcttgtttctcaccagtgagcttaacctgggacacaaggtaccactgaaacaaagagaacaatattcccacaacagcacatacaggaatcaagatctctgtaccaagatctgataatattgttgctgacgtcattagatctgattggtttgttccaattggtcttgaaaacacaagatcaatggaagggaaggaggtgaggggtttaatg

SEQ ID NO: 107 - DNA, 2005 bp, Artificial Sequence
  Synthetic Construct
ttagccaggagcttactttcactacttatttagctactttaggtcgttgtagtccataaataaaaagaaaactttatcagagcttgcattcgatgccgttgattcaatttcttcaccaccggtagcaatcaattctaaaaagaagcttccggaaagatattctagtaaatagaaaactcgccattcattcatgaactgtcttgcccgtgccatcaagagctacagctatatctctcgaaggtgtcttagctccaccgataggaactgcccacctatccaggtagcctcacccgtgaaccacgaactacacttacagacaacctaagaagaaacggagtctcacccatcataagcataagaagcaaaaactagaagaataaacaagagataacgcgcatacaaagaggattcttaaccgttcgcgaagagaagatagttgttttaggcaaggaaaaaagccaaccatagctgagcgcaagcaaagctttcggagattattttatttaaaaaattagaaaaaaaaaacttaaaaaaaaacccagcgaaatattgatttgtctgatttgccaaaaaagcctcaccacaggattttgttatctagttaacagccttagcctgaaagtgttcattatcactagttttaagtctgtatacttggatccttcttcaacaccacaaacaaacttctgctcttattttctttacttcttccattcattcatcatcaatggtggtctgactcaactgttaggaataatgacatcttcaatgctgtatggagctgaaaatctggcaataataagaggaagagttggtgcaaatgggttaggatttaatgggtcagatttgaatgggagaaaatttccactcaaagttaatttagcttgtcggagttcggtttcgagaggtagaaccttagttgtacctaagtgcagtgtgtctttacctaggccagcttcacaacctaggttcatacaacacaaaaaggaggcattttggttctataggtttttgtctattgtgtatgatcatgtcataaacccgggccattggactgaagatatgagagatgaagcgttagagccggccgatctttatagccggaatatgatagtggtggatgtcggtgggggtaccgggttcactactttgggtattgtgaaaaatgttgatgctaaaaatgttacgattttggatcagtcgccacatcagcttgctaaagcgaagcaaaaggaaccgttgaaagaatgcaagatcattgagggtgatgctgaggatcttccttttaagactgactatgccgatagatatatatctgccggaagcattgagtactggcctgacccgcaacgaggcatcagagaagcatacagggttctaaagataggagggaaggcatgtttaatcgggcccgtatatccaacatattggttgtctcgtttctttgcggacgtgtggatgctttttccaaaagaggaagagtacattgagtggtttacaaaggctggatttaaggatgtgaagattaagagaattggcccaaaatggtatcgtggggtccgtcgccatggtttgatcatggggtgctctgttactggagtcaaacctgcatcaggggattcaccgttgcagttgggccccaaagaagaagacgtctcaaaaccagtgaacccattcgtgttccttgggcgttttctcttgggtgcgttggctggagtctactacgtcctggtccccgtctacatgtggctcaaggaccaaattgttccaaaaggtcagcccatctgagtgatggtcaagtcacttttggtttgtatcaaatgatgtccagttttcaatatcatgtgtgttatgtgaacccctgttaggattttagccctttgtttttttatgaatgtttgaccataaactttcataatttgtgcaattttttgtttgccaataactctcatacacttcttttgacgaggcttgaacccacacccttttattgacaagagacttg

SEQ ID NO: 108 - DNA, 861 bp, Artificial Sequence
  Synthetic Construct
gcatcttttgtctccaacaaaccacctagaagcgataactgaaacggattcattgctgcagatgtcatgatataccatctacattatcagtgattgcatttttagtttaaagttactgtgtatgttctgaggaattgtgtgtaactgaaactaagatgtgttgtgctaatgaataatatcttagacgacgctattcaatttgcttgctgctaaatttaaatctttgttataactaatcggtttacataatccattgatggaattattttatttgaaactttattataagacctcataacgtcacaataaattttctatgctttgcgcagattttctggctcggctactgttgttaagctgcaacgaacaaagttgtatgcatagtaaatagaacaatacaggcaatgtgaggttgctaacttcagtttatgactgcatgtctgcatccatgagcattttcttttgtactagctgttaaacttggaaacgagcaaagagactcacgattctgatcaaaacttttgatcgactctttttattaacttgaattaacttgaatgaataaaacattacaaccatggggtttatatagactagctattctaatcctattagaactctaatactaaacataattatctaaacattaggaatcataattcaaacctaatactaacactaatagataattcaaataataattaaacaaacttaattattattttactcaattaattagtccttcgactttagcccttcgaattgatattgctacaattttcacaggatacgtcgtttgtctttctgatgatttataagttagatgaatcctttacttaccaagatatcgagttttttgaa

SEQ ID NO: 109 - DNA, 762 bp, Artificial Sequence
  Synthetic Construct
aaaagaactcacttagtgatagaactagaactttttagatgacaagttatctagttttcacttactacaactagaggctccatctcattatttattaatacaaaattttcaaagatacataaaattctaaccacattgttgacaggaggttaacaaggatcctcttgaccccctttggttcccgcccctcctatgattagtagcatttcttgatatacgaatcactatgaataacgaagaatacgacaacaatgatacccaaacaaaagcatgtgccattaatacttcatagtgacaaccttcaacacattagacaaccatggatgtaacccacaaatgcatgtggatatatccattcttatttaacacacaaacataaaaacacaatacataattcaaaatacactgttgtgcttgtttgggcttctgctttcatcttaatctttagcagctgcatttgtcaccacacttgcagttgtcaccacacttgcagtcgcagtttccaccctcagcaacaaagctgccctcacttccctcagcaaacatctgtttaggggcaacaccatcaacgatcatgacagtggtggccgaaacctcaacatcagggtacatcccacatccgttgcagccgctaccgcatgagcagcttgagccacacccgcactttccactgcagcaagacatgattgagtgtgttaaagagggaatatttgaagggtttagttgtgtgtttttgattgaatacgatgaagcttgatggtc

SEQ ID NO: 110 - DNA, 770 bp, Artificial Sequence
  Synthetic Construct
gtgaactgtcgcaacatattagcacggctggcatggaagctagtggtacaagtaacatgaaatttgcacttaatggatgcttaattataggaacattagatggagctaatgttgaaatcagagaagaagttggagaagaaaactttttcctcttcggagcaacagcagatcaagtcccccagctcaggaaagaccgagagaatggactgttcaaaccggatcctagatatgaagaagcgaagcaatttattagatcaaaggcatttggaagctatgattatgaaccacttcttgattctttggaaggaaactctgggtacggccgtggcgactactttcttgtcggtcatgacttcccaacctacatagacacacaagctaaagtggatgaagcatacaaggacaggaagaaatggataaagatgtccattttaagcactgcaggaagtgggaagtttagcagtgaccgaacaatctctcagtatgcaaaggagatctggaacatagaggagtgccctgtgccttgattgcatgcatatctccattggcaaggttggatataataataagtttaatgtttatattatttgtgtttacattccctgaaatatttttctgtattatgtaaaaatcacgcacttgttacaacttataatacgcgtaatttattacttttcaactccttgtctttgggttttgtgtgatcttgatccgattttcaggttgcattgtatatacaatggtatcttgatccgatttttaggtcgaac

SEQ ID NO: 111 - DNA, 1593 bp, Artificial Sequence
  Synthetic Construct
tgcagcctttaaatctggtttaaaaacttattcgcaaataaaaatagaaaaaaattaacaaaactacaattatttgttttcatggtattaacaatttacagaacgacatcataaatagaaaatagagttcacaatagcaaacaaacaaccagagttcacacagaagtagcagagctcttaaatagcttagattacacatacaaataaatatgcatatgcataaacacaaacatgaaaacaagaatcttgaatttaagacatcataatatcacactttactctttgccatcagcagcacggagcgatcccagttgaaccggtgcctgggtcccaacgactttagatgacccgtaaaccgagaggtcaaagccttcacctctttctttctcaatttgttccttgatccagaaatatgtgattctcaatccatcctttaatctcatagtaggggcccacccaagcttctctttgatcaaagtgttatccgagtttctaccacgcacaccttctggacccggaatgtggtgaatcggaagcttcttatcctcaaaactgagaacaatttcagccatttcattcatgctcaccatctcatcacttccaatgttcaccggttcacggaagtcagattttgtcaaccttaaaacaccttcaacacattcatcaatgaaggtaaaagatcgggtttgtagcccgtctccccacatttcaaacttatcagtcgccgtaagcgcttttctgcagaatgcagcaggagctttctccctcccacctttccatgtgccgaatggtccatagatgttatgaaaacgtccaatacgacattcaattccaaagtctttagtgtagtgcctgcacaactcctcagttgctagtttctccaaaccgtaagcatcttgaggctcagcaggccacgcgtctgactccttcaagctcacattggtttccaactgcttaaactcagggtaaatgcaagcactagaggcatagaaaaacctcttgactccactgatcctggcagcctcaagcatgttaaagctaatcattgtgttattatacataataacagagtgatttgactgaatgaaccccataccacccatgtcagcagccagattaaagacatgatcaactttttcagtcactttcaagcaattatccatcacgcgaagatcaacaagatggaactcatgacaaaacatgtcttctgacatgtgctcatttttcttccaatcagaagcaatgatgtagtggccctcggtttttaaacgcctagcaatgtgtgatgcaatgaacccacccgctcctgttatggaaattctaagttttgctgatggccaataaggttccctctcaaggttttcataggtgaatgacccgtattttgtttcaccagcggttcccattctgataaaaataaaaactgttgaagggatatacccaagatgatcccttttaaagtgggttagatttgctttcacaaatctttcagtgagaggatttgtatgttactctcgtggtggaatacaatccagttatccttgtgctgcttcatggtccttttatttaggttttccacctccggattttgca

SEQ ID NO: 112 - DNA, 1126 bp, Artificial Sequence
  Synthetic Construct
ctcgtttacaccctttttaagtttggtttattctcaacacccctagaccctagtatgcctaaaaatgtatcaagttccaatatattttgagtaacgcaatatatcaaactcaaatcatttatcaattttcatacacatattcttgtcggcatcatatatagatatttattaacacaaacataacccatcttgatctttatattcttatctccccacatcatatcacaccactcactccacactctcaccaaaaccaaaccatgggcgtaataataatcgacggttccaccgtacgcgccttcgcgaacaacgaatcctacttcaacaccatagtcgacgcccagttcacctctctggacgtcaacaacgacggcgtattatccaggtcagagatgcgcaaagccttcgaatccatgcggttactggaaacccacttcggcgtcgacactgcaacgacgcctgaggagctgacacgtgtctatgattccgtgttcaagagctttgacggagaccataacggcaccgttgacccagaagagtttagatcagagatgaagaagattatgttggctgtggctgatgggcttgggtcgtcgccgatacagatggcggtagaggatgatgaccagagtttttttgaagaaggcggcggatcttgaggcggcgagcggtggtggttgaccggactgggtatgggataagattttacaaccggaataaacggatccattaatatctctagttatgtgatgttaatgaataataagttaagtaataataacttgtgttgtttttacttaaagttaaaaagggtaaaacggtaaaagagtccatgtggtgtactggtgtgtatgtgtactgcggtagtgtacttggtttaattttgttactttgctcgtttgttactttaatcatgtggtttttgcgacttgaacccaaaacctcctgtaacctaggtacctcgtgaaaatcaagagtttggaacctatcttggttagaaaaaataaataatcgaaacaagtgaaaaacttgaagaactgatagattcgttaaagttatatgttaaataactgaatcgacattaaaactgagcttaatgtttttagtttcatgaatgatatgctga

SEQ ID NO: 113 - DNA, 1604 bp, Artificial Sequence
  Synthetic Construct
tttctgtaactttgacttggagttatttgtaccatagatcagttgttttcatggggcgcttttgaagtttttttttttttttttttaaattttaattagacaagagacataggaaaacttttttttggcaaaaacatgtatcacgccccttgcttcttgcttgcttcagatttcttctctttactaaagaatgagatttgagttccagtgaatttcggtatgttattgaaatatcagtttctaattcttatagcatctcacttgttcatcaggttgtctgttcaattgtctgacgaccgattcaaccaacatgtagtaggaatgtgcaagaaacttaaggccgaccttaagaaatggtaaccaacattgtagcaggagtggatggtaatatgttacttggcagctataatcatgaggagatttcaatcgattgttttatttcaccagaacagccatgtttaggatcatttattgtctatgttgtgaagttttgttctctgctgtcccttttgatcggttcgtatatcaatgcaattcatttttacatgtaaaaaagatagaccgccgtctaacgtgtagcgtatttcatgtaacttgatgctagatttcatttttgacttgcaatcctctattcaagtctgtagactgtggtcgcgtactcgcgtggatgaaatatgggtatggttatgtagtaggagtggagagtaaaatagtcatgacttgataaataatactttaccaagattattatcatttaattctgattaaactgttgatttgtatatatgtatgtttaatgcatttgtgaggagtatttggaaaacagaacaaaatgctacagtgatgtgtagcctgcactttatcgccttgtgaagaataaaaatgctactgtgacagagtcgtgggaatgtgcaggcgtccatcttctgcagatgaactgaatgagacggttcagcggcgtactcagttgttttgtgttcagtttaatgatcggcaagacgtttggcgttgggaaggcaattctcaaggagtttttttcggtagccgactataaaagactctttgctgcaggaagggatcagcagcgacatgttagatgtagtggatttcatgggtttttcttaaggttaaagtttttctttggcgcgccggattaaaccaaattcttacccgtgttatatcagacgattcctgcgttttgtgtgaagctgacccagtaacggccctacttatttttaccggatgcggatattcgtttggtgtgtgggcggcaatcggatcttggtgcaagatcgcttctctgtatgcttttgatttgaaagatttgttgctgattggagatgggaagagaaataaagacacaaagaagattatcaatgggattatgataataacactttggacgatttggaatacaaggaacgataaattgtttagaaacaaagaggcgaaagtgagcgaggtggtcgcaaaggttaagtttgtcttttcttgtggattaggactcgttctagatttaaaaaattagtctagaaagagtggtcgatgtatcctttgtatatgttgtagttcggtgtgcggcggtttcctcgccttgagggccg

SEQ ID NO: 114 - DNA, 464 bp, Artificial Sequence
  Synthetic Construct
ttacatctctaaacctagagagggtatgctcttcatatacatacattagtcacccttgaaataatctaataaacgttaattattacatataatatccaatgtataagaacaccaaaggatttttaacatttgaataacatattcaaaacctaataatatacatgtaacattaacctaaatttaactttacatttagttcaaataccataataataaaatgaaaggaaacccttgacatttttaatttttgagacctgcagggaaacaaggttgattctgcatttgctgaaacaaagcagccattctactgtaactttccatcgtcattggctgtgactggcatgcaaggaaggcagccatgggatcattttgatttggaactcgatcgccagtggccgaatgcatatcccacgaagccatcatcggcggcacaaaggtggaggggtggaagaccggtggcattc

SEQ ID NO: 115 - DNA, 1979 bp, Artificial Sequence
  Synthetic Construct
ttttggaatttttgctaattgtccacacgacaaaacaaagaaagaaagatggtgttaacaaccacctcatgtctccaccctcggccaccaattgcccctcccaccaccactcacgcgccaccgtccgccagaccaaagagggagatttcttggaggaaacaatgtgtagttgggatggcatgtgtgataattggtctacaagtggtgggcatagtgggagatagccatgacctcgcgatcgccattgagccaaaagccgctatagagtcaaaggcaaagggaaagaggtggagtaacaaaagaatatgtccggcttggcaattgaattcattggagaccatagtgcccgaagatctcccacgaccatcatccaagaggcgttgggaggcggttggtgatcatttaagagttgtgccgccacataaatcttccaccaccaacatcactatggcgggtggtgattgtttcactttgtagatactagatcacttgatagatgtctatatacttatggctgtaaatatcatacaacatacatatttgtatcaaatacttagcttggaacattattcaacacattcttagtgacaaaaattgaaccttttttctttctttcttgattgaaactcaaaaagggtttgatgtttatgtcatgaactcaatagatcatacgtttgtgacgcgtgtatgtatacatatttaatgaagtgttttatatgtaagcctttgtgattcgcataagtttattggatacctttcgggtcactataaccctatcacttcatggcatacgtgacgcgaaaaatacactagcccaaccttaaccgaactatagtctaaccttctatgaattcatcttaaatttatgacaccgccatccttgacattcttttccctgtcatcagacccctttcttgacacgcaatgcgtgtcaagaaagggcttttttctagtagtaccattaccggaccttttgcaccttcttccacagtcatagctccagtttgagctgtcatatctgttataacatacccgggatgcacacaattcacaagaatattctcatgttttcttgccatgagccttgtgtaagcattaagagcaacttttgaaactttgtaggcagagactgttagtggccatccattctcctttaacttaccagccttaaaatcactcaaaaaccattggattatctcatctatcctctcttctgataagttgtcaatatctagcaactcttctttcaacttttcattgtggaaccagtataggtcactgtagagagagctaatgttcacgattcttggagatttagagagtttgagaagtggaatgagcgaatctgtaagccttttggtcccatagaaatttgttttcaggcatttttctcctagttcgtaaggttcttccagaatgttggcaaaaagatgcgccttctcatcgacgacttgtagaaaccctcctccatctctaaattctttttcagcaagtattataattccaagttccgctgcattattgatcaagatatctagcttcccaaagtgtgtttgaacaaatttggacaaattggcgatgctagatgggtcgttaatatctagttgatgaaaaacgacgtttgaaagaccagagacgttaagtttctcaacagcttcttccccgcgactttgatttctagatgttaatatgaccataactccatttgaagcaagttgacgacatatttcaagtccaattcctctgtttcctcccgtgacaaccgcacatctttgttcctccattgaaaagtaggaagtgaaattgagatatgaagaagtaagcaatctctcctttttatagtgacttgttgaaacatataaaaccactcccacgtccccaccacaatatcaactttaacgttttgtacaattttgtatgtgctaaagcctaaaagcattcaacgtatgaatatcatataatatatgaattattggaag

SEQ ID NO: 116 - DNA, 208 bp, Artificial Sequence
  Synthetic Construct
gtaacatagcatagcatgcctatggaaaacagcccctcccacttatacactccacctcatcattacatttaagaaagatggttggttcccacataatattcacccacaaacctcttcatacatttttatcaactttctttcttggcctttctaaaatattattcaaactttaataatctttttctcttcttttatttggctactcttc

SEQ ID NO: 117 - DNA, 736 bp, Artificial Sequence
  Synthetic Construct
aacaacttccaaggagtgaccgacaagctaaatcacaggtttttgtttgaacatattcattttaagaatatggtccttcaagctgagcgatacaacggtggctaccagcagcagagctattcatcataccaagggccaactggcggctacaacaacttccaaggagtgaccgagtacacatccccatatcctggctcttttcaagtgtcaacttacccatcgagccatggtccacactaccaatctggttggtctagtggcggtgactactatggcaatggacccaatcactaccctggtggcggcatgcttcctcatccagtgaagcccatcactaacactcatccggtgaagcccaccaccaacgactatgaaagttctttctatgagagctcatacacccatgggagcccaaaccatcatggccatggaagcccccatgatcagggccatggaagcccaaaccatcacggctatgggagcacccacggtcagggccatgggagcccacaccaccagggccatggtagcgccccacatggctttgggaaaacacaaggctttggaggtgctataactaacaaaattaacaagctgactaatggctataccaactatggaagtggtagcccaaaccatcatggccatgaaagcctaaaccatcatggccatgggagcctaaaccaccatggccatgggagcccagaccaccatggccacgggagcccatatggct

SEQ ID NO: 118 - DNA, 1695 bp, Artificial Sequence
  Synthetic Construct
aaaatccaaaaacgaggagaattcaaaatgaggctcactggagttcttatctgctgctagtgggacccagatgatcacccgtgcatcttataaattcaattctcctcgtttttggattttaatttcatcttccttccacacaattttcatcaatcatcaggtggagtagagatggactatacctatgcaccaggcaggaatcacctgtttgttccgggacctgtgaatatcccagaccaggtgcttcgtgctatgaatcgaaacaatgaggactatcgatctccggcaatccctgcattgacaaaaactctacttgaggacgtaaagaagattttcaagaccacttctgggactccttttctcatcccaacaacaggtaccggtgcatgggaaagtgctttaactaatactttgtctcctggagaccgaatcgtgtcattcatcatcggtcaattcagtttgctttgggtggatcaacagaaacgccttggcttcaatgttgatgtagtggagagtgaatggggtcgaggtgctgacttggatgttctagcatcaaaactggcagcagatactgcgcatactattaaggctgtctgcatcgttcacaacgagacagccactggagttaccaacaacttggctactgtaaggaaaatacttgatgagtaccagcacccagcgctgtttctagtcgatggtgtgtcatcaatctgtgctctggatttccgcatggatgaatggggtgtagacgtggctttaacaggctcccagaaagcactttctctaccgactggtatggggatcgtgtgtgcgagccccaaagcaattgaagcttctcaatcagcaaaatcacttcgggttttcttcgactggaaggattacttgaaattctacaagatggggacttactggccatacacaccatctatacagcttttgtatggtctaaaagcagctcttgacctcatttttgaagaaggattggataatgtcattgcaaggcacactcgtcttggaactgccacaaggctggctgttgaagcatggggtttgaagaactgcacccaaaaggaggaatggttcagtgatacagtcactgcagtggttgttcctccatacattgatagttctgaaattgttaggaggggatggaagagatacaacttaagcttgggtctcggcttaaacaaagtggccggaaaagtcttcagaatagggcatctcggtcacctaaatgatttgcaactgctggggtgtcttgcaggggtggagatggtgctcaaggatgtgggctacccggtgaagcttgggagtggagtggctgctgcttgtgaatacttacagaggagcatcccaatgattccatcaaggatttgaattctaaacatgttatgtttgtgtttatgtatgttgtttcatactaactttggaatgcctagacaataagtgatgatggttatgtatggcagtgaatgaaacaaataacacgaaatgaattattaaaagctgtatgtaaacacatacttgtatgattaaattgtggcgtgttaaacaaaacaacatccccaaacgtgagataattcctcatttcatcctcatgaaataacaatccagtagtccccatcaatgtactgtattcatatgaaaaatgagatagtgtttggtatgcacgagtgagaaacgaaatgaaatgag

SEQ ID NO: 119 - DNA, 1723 bp, Artificial Sequence
  Synthetic Construct
ttccattccattccattccattcacctcacgtaataataataataatattattatcttgagatccagtcttcttaattaattagcgaaaatgagagaatgcatttcagtccacatcggtcaagccggaatccaagtcggaaatgcttgctgggaactctactgcctcgaacacggcattcagcctgatggccagatgccaagtgacaagacagtcggtggaggtgatgatgccttcaacacctttttcagtgaaactggtgcagggaagcacgttcctcgtgctgtatttgttgatcttgagccaactgtgattgatgaagtgcgctgtggtacttatcgccaattgttccatccagaacaactgatcagtggcaaagaggatgccgccaacaattttgcacgtggtcattacacaattgggaaagagattgttgacctctgcttggaccgtatcagaaagcttgctgacaactgcaccggtcttcagggtttcttggtctttaacgctgttggtgggggtactggctctggtttgggctccctgcttctggaacgactctctgttgactatgggaagaaatctaagcttgggttcactgtctacccatccccacaagtctcaacttctgttgttgagccgtacaacagcgtcctttcaacccactcactcttggagcatactgatgttgccattcttcttgacaatgaagccatttatgatatctgcaggcgatctcttgacatcgagcgtcctacctacaaaaaccttaatcgtttgatctctcaggttatttcatccttgactgcatctttgaggtttgatggtgccctgaatgttgatgtgacggagttccagaccaacttggttccatacccgaggatccacttcatgctttcatcttatgctccagtgatttcagctgagaaggcatatcatgagcaactatctgttgctgagataaccaacactgcctttgagccgtcttctatgatggctaagtgtgacccacgacacggaaagtacatggcatgctgtttgatgtaccgtggtgatgttgtcccgaaggatgtgaatgctgcagttgcaaccatcaagacaaagaggaccatccagtttgttgattggtgtcctactggtttcaaatgtggtatcaactaccagccaccgactgtggtgcctggtggagacctggctaaggtgcagagggctgtttgcatgatctcgaactcgactagcgtggctgaggtgttctctcgcattgaccacaagtttgacctgatgtacgctaaacgtgcttttgttcactggtacgtgggtgagggtatggaggaaggagaattctctgaagctcgtgaggatcttgctgctttggagaaggattacgaagaggttggtgctgaaggtggtgacgatgaggaggatgaaggagatgagtactagataatataatggttgtgtttgtgtttggattgtgatggatattttcttgtgtttgttgccatgttactgttagccgtatctttcttgtttcaaaactctgtattgtcatgtttgatttaatcttgtggagttatgctatattatgtcgctattatatggatttaaacgtctttagtatcctgatccatgcacaatgttattaatcatgttctttaaaaatgtaattgacaaaacagataaacgatgcgtatgcaatcgctatcttgtttgtttatgcaaaa

SEQ ID NO: 120 - DNA, 2209 bp, Artificial Sequence
  Synthetic Construct
ttcttcataaaccctaacatttcttatctcaaagcctcacttaacacaactcacacctactctctccctctctgttatcttctcctcctttcctctctgtttttccatatcttcagttaattggttgaggattgtaagaagatggcatctattactcccttatcttccataacctcttttggaactcccaagacaattgcaagttcgtcaggaacattgtcttcattgtcttctatctcgtctactcgtcttgtacacggtaggcaaaatgttgttattcagagaaaaaaattctcttcaggttaaggcaatggccaaagagttgtactttaacacggatggttctgctattaagaagcttcaggctggtgtcaacaagcttgcagatcttgtgggagttactctaggtccaaaaggacgaaatgtggtactagaaagcaaatatggttctccgaaaattgtaaatgatggtgtgactgtagctaaagaggttgaactggaagatcctgttgagaacattggtgccaagttggtgagacaagcagcctcaaagactaatgatttagctggtgatgggacaacaacatctgttgttctagcacaagggttaattactgagggtgttaaggtggtggcagcaggcgctaacccaattcagattactagaggtattgaaaagaccacccgagctttggtggaagagcttaaacttatgtcgaaagaagttgaagacagtgagttggctgatgtggcggccgtaagtgcagggaacaactacgaaattggaaatatgattgctgaagcaatgagcaaagtgggaaggaaaggtgtggtgacacttgaagaaggaagaagctctgaaaactacttgtatgttgtggaaggcatgcaatttgatcgtggttacatttctccatacttcgttactgatagcgagaaaatgactgttgagtatgataattgcaagttgcttctggttgataaaaagataacaaatgcaagagatttagttggtgtattggaagatgctattaaaggaggatacccgatattaattatagcggaggatattgagcaagaggcacttgctactcttgttgtgaacaaacttagaggttcattgaagattgctgcacttaaagcacctggatttggtgaacgtaaaagtcaatatcttgatgatatcgctatcctcactggaggaactgtaatcagagaagaagtagggttgaaccttcagagtgctggaagtgaagttttagggcttgctgcaaaggtggttttgaccaaggattcaacaactattgttggtgatggcagcacccaagagttggttaataaacgggttgctcagattagaaatcttgttgaggcagcagaacaggaatatgaaaaggaaaaattaactgaaagaattgccaaattatccgggggagttgccgttatacaggttggggcacaaacagaaacagaactcaaagaaaagaaacttagagtagaggacgctctcaatgcaacaaaggcagctgttgaggaaggaattgtagttgggggtgggtgcactctattgagacttgctgctaaagttgacgccattaaagccacctttgacaatgatgaacaaaaggtaggagcagatattgttaaaagagctcttggttaccccatgaagttgattgcaaagaacgccggtgttaacggaagtgtggtgattgaaaaggttctatctagtgacgacttcaggtttggttacaatgctgcaactgggaaatacgaggatttaatggcttctggtattattgatcccacaaaggtggtgagatgttgcttggagcatgcatcatccgttgcaaagacattccttacgtctgacgcagtggttgttgaaatcaaggagcctgaacccgctccccttgcaaacccaatggacaactcaggttatggctactaagctgcagctagctagcaaatagaatcttaaacctgtgggagttctttgcatacttctaatcaagaaatgttagcttccaccacgtttctttctattagctctattttatgactgtaaggaagtttaaaccctttttccaatatatttggaggtcgttgagtaggttgaattttggcgggaaaaaggtttgtaggattggtatttgtattcaatttgataactgcattaataagaaagcgatattgtctaaaaaaaaaa

SEQ ID NO: 121 - DNA, 21 bp, Artificial Sequence
  Synthetic Construct
accgaacaaccacaaggtcaa

SEQ ID NO: 122 - DNA, 20 bp, Artificial Sequence
  Synthetic Construct
gggctgcataatgagcatca

SEQ ID NO: 123 - DNA, 20 bp, Artificial Sequence
  Synthetic Construct
ccgaacaaccacaaggtcaa

SEQ ID NO: 124 - DNA, 19 bp, Artificial Sequence
  Synthetic Construct
gcgattgggctgcataatg

SEQ ID NO: 125 - DNA, 21 bp, Artificial Sequence
  Synthetic Construct
cgaacaaccacaaggtcaaca

SEQ ID NO: 126 - DNA, 21 bp, Artificial Sequence
  Synthetic Construct
catccactctccgacacaaca

SEQ ID NO: 127 - DNA, 20 bp, Artificial Sequence
  Synthetic Construct
ccctagctccaacgcatcaa

SEQ ID NO: 128 - DNA, 22 bp, Artificial Sequence
  Synthetic Construct
ggagtctcttaacggcggataa

SEQ ID NO: 129 - DNA, 19 bp, Artificial Sequence
  Synthetic Construct
acccccaccccttctgatc

SEQ ID NO: 130 - DNA, 21 bp, Artificial Sequence
  Synthetic Construct
ccgacgaggttttgactggat

SEQ ID NO: 131 - DNA, 19 bp, Artificial Sequence
  Synthetic Construct
ccatacccccaccccttct

SEQ ID NO: 132 - DNA, 20 bp, Artificial Sequence
  Synthetic Construct
cgttttccgacgaggttttg

SEQ ID NO: 133 - DNA, 19 bp, Artificial Sequence
  Synthetic Construct
ttcgcgggtgtaccaacaa

SEQ ID NO: 134 - DNA, 21 bp, Artificial Sequence
  Synthetic Construct
cagaaagccacttgccctaaa

SEQ ID NO: 135 - DNA, 20 bp, Artificial Sequence
  Synthetic Construct
cgcgggtgtaccaacaacta

SEQ ID NO: 136 - DNA, 20 bp, Artificial Sequence
  Synthetic Construct
caaaggcccctaagccacat

SEQ ID NO: 137 - DNA, 22 bp, Artificial Sequence
  Synthetic Construct
gcgggtgtaccaacaactaggt

SEQ ID NO: 138 - DNA, 21 bp, Artificial Sequence
  Synthetic Construct
ggcccctaagccacataaaca

SEQ ID NO: 139 - DNA, 20 bp, Artificial Sequence
  Synthetic Construct
gcaacaccaccatgcatgag

SEQ ID NO: 140 - DNA, 22 bp, Artificial Sequence
  Synthetic Construct
aactcccatcaattcccacttg

SEQ ID NO: 141 - DNA, 20 bp, Artificial Sequence
  Synthetic Construct
cggtgctacagaacccacaa

SEQ ID NO: 142 - DNA, 20 bp, Artificial Sequence
  Synthetic Construct
ttgctggtggcgttcctaac

SEQ ID NO: 143 - DNA, 21 bp, Artificial Sequence
  Synthetic Construct
acggtgctacagaacccacaa

SEQ ID NO: 144 - DNA, 20 bp, Artificial Sequence
  Synthetic Construct
atcaaggcctcatgcatggt

SEQ ID NO: 145 - DNA, 21 bp, Artificial Sequence
  Synthetic Construct
tgaggaaaatgcttccatcgt

SEQ ID NO: 146 - DNA, 21 bp, Artificial Sequence
  Synthetic Construct
gacagacatcacccgcctaac

SEQ ID NO: 147 - DNA, 22 bp, Artificial Sequence
  Synthetic Construct
aagttaggcgggtgatgtctgt

SEQ ID NO: 148 - DNA, 21 bp, Artificial Sequence
  Synthetic Construct
cgctgtaagatgcacggagaa

SEQ ID NO: 149 - DNA, 21 bp, Artificial Sequence
  Synthetic Construct
gggacttcagctgtgatttgg

SEQ ID NO: 150 - DNA, 22 bp, Artificial Sequence
  Synthetic Construct
cagacatcacccgcctaacttt

SEQ ID NO: 151 - DNA, 24 bp, Artificial Sequence
  Synthetic Construct
ccttgactttcttggcgtagtaca

SEQ ID NO: 152 - DNA, 20 bp, Artificial Sequence
  Synthetic Construct
gcgcgggttaaaaaaaacct

SEQ ID NO: 153 - DNA, 22 bp, Artificial Sequence
  Synthetic Construct
cccttgactttcttggcgtagt

SEQ ID NO: 154 - DNA, 17 bp, Artificial Sequence
  Synthetic Construct
ggtggcgcgggttaaaa

SEQ ID NO: 155 - DNA, 22 bp, Artificial Sequence
  Synthetic Construct
ctcgaacgctaagctcatttca

SEQ ID NO: 156 - DNA, 23 bp, Artificial Sequence
  Synthetic Construct
atcacagcatatccacgcctaga

SEQ ID NO: 157 - DNA, 21 bp, Artificial Sequence
  Synthetic Construct
tgaagctgcttagcgaattcc

SEQ ID NO: 158 - DNA, 22 bp, Artificial Sequence
  Synthetic Construct
acatctctgacatccccgatct

SEQ ID NO: 159 - DNA, 20 bp, Artificial Sequence
  Synthetic Construct
ctgggcctcatcagctaagc

SEQ ID NO: 160 - DNA, 23 bp, Artificial Sequence
  Synthetic Construct
gagcattacaagaagtgcatgga

SEQ ID NO: 161 - DNA, 23 bp, Artificial Sequence
  Synthetic Construct
gagttgaagctgcttagcgaatt

SEQ ID NO: 162 - DNA, 20 bp, Artificial Sequence
  Synthetic Construct
catctcccccaatccaacat

SEQ ID NO: 163 - DNA, 21 bp, Artificial Sequence
  Synthetic Construct
ccctcaaaagcaaagccatct

SEQ ID NO: 164 - DNA, 21 bp, Artificial Sequence
  Synthetic Construct
acgttcctgagcttgctgaga

SEQ ID NO: 165 - DNA, 22 bp, Artificial Sequence
  Synthetic Construct
tggtgatcagcaggaacatacg

SEQ ID NO: 166 - DNA, 20 bp, Artificial Sequence
  Synthetic Construct
tgggctgctttggaaaaact

SEQ ID NO: 167 - DNA, 20 bp, Artificial Sequence
  Synthetic Construct
accagcacgctccacaagtc

SEQ ID NO: 168 - DNA, 18 bp, Artificial Sequence
  Synthetic Construct
cggccagatcccatcaca

SEQ ID NO: 169 - DNA, 25 bp, Artificial Sequence
  Synthetic Construct
caaaccagacaagttcacttcagaa

SEQ ID NO: 170 - DNA, 21 bp, Artificial Sequence
  Synthetic Construct
cataaggcggtggaaggattc

SEQ ID NO: 171 - DNA, 22 bp, Artificial Sequence
  Synthetic Construct
ggtgaaaccacttttggaaacc

SEQ ID NO: 172 - DNA, 26 bp, Artificial Sequence
  Synthetic Construct
cagcagttctttgttttcttttcagt

SEQ ID NO: 173 - DNA, 22 bp, Artificial Sequence
  Synthetic Construct
ttccaaccatttagtgccacaa

SEQ ID NO: 174 - DNA, 28 bp, Artificial Sequence
  Synthetic Construct
gtatgtattttggtgtgcatatcatgtt

SEQ ID NO: 175 - DNA, 22 bp, Artificial Sequence
  Synthetic Construct
catgtgcattcagggactcgta

SEQ ID NO: 176 - DNA, 19 bp, Artificial Sequence
  Synthetic Construct
tcaccgctgatcccaacaa

SEQ ID NO: 177 - DNA, 20 bp, Artificial Sequence
  Synthetic Construct
ggccgacagacgtacgttct

SEQ ID NO: 178 - DNA, 23 bp, Artificial Sequence
  Synthetic Construct
cttctcaggctgaaactggttgt

SEQ ID NO: 179 - DNA, 20 bp, Artificial Sequence
  Synthetic Construct
caccaaacaggcatgtgcat

SEQ ID NO: 180 - DNA, 20 bp, Artificial Sequence
  Synthetic Construct
tctgcatcatcaccgctgat

SEQ ID NO: 181 - DNA, 20 bp, Artificial Sequence
  Synthetic Construct
ccctctgcccctaccgtact

SEQ ID NO: 182 - DNA, 20 bp, Artificial Sequence
  Synthetic Construct
tcaaatcccagggctcaact

SEQ ID NO: 183 - DNA, 21 bp, Artificial Sequence
  Synthetic Construct
gccacctacagacgctttacg

SEQ ID NO: 184 - DNA, 20 bp, Artificial Sequence
  Synthetic Construct
agcagccgcggtaatacaga

SEQ ID NO: 185 - DNA, 20 bp, Artificial Sequence
  Synthetic Construct
ctccccaggcgggatactta

SEQ ID NO: 186 - DNA, 22 bp, Artificial Sequence
  Synthetic Construct
agtcctagccgtaaacgatgga

SEQ ID NO: 187 - DNA, 21 bp, Artificial Sequence
  Synthetic Construct
cagggttcctgctctcagctt

SEQ ID NO: 188 - DNA, 20 bp, Artificial Sequence
  Synthetic Construct
gatttgtccggctgttgtga

SEQ ID NO: 189 - DNA, 20 bp, Artificial Sequence
  Synthetic Construct
atgtgccacagcatcaatgc

SEQ ID NO: 190 - DNA, 22 bp, Artificial Sequence
  Synthetic Construct
gcattggatgaaccttgaagct

SEQ ID NO: 191 - DNA, 21 bp, Artificial Sequence
  Synthetic Construct
tgggacgaatgatgaatttgg

SEQ ID NO: 192 - DNA, 20 bp, Artificial Sequence
  Synthetic Construct
tttgtccggctgttgtgatg

SEQ ID NO: 193 - DNA, 18 bp, Artificial Sequence
  Synthetic Construct
cgcgggtggattcatgag

SEQ ID NO: 194 - DNA, 21 bp, Artificial Sequence
  Synthetic Construct
cctgacgtaacgagcaccatt

SEQ ID NO: 195 - DNA, 20 bp, Artificial Sequence
  Synthetic Construct
ccacggcgtcaaaaagctta

SEQ ID NO: 196 - DNA, 20 bp, Artificial Sequence
  Synthetic Construct
ccacggcgtcaaaaagctta

SEQ ID NO: 197 - DNA, 20 bp, Artificial Sequence
  Synthetic Construct
gatcctctgtggtggttgca

SEQ ID NO: 198 - DNA, 22 bp, Artificial Sequence
  Synthetic Construct
gacgtaacgagcaccatttcaa

SEQ ID NO: 199 - DNA, 21 bp, Artificial Sequence
  Synthetic Construct
gttccagagcatccaatgcat

SEQ ID NO: 200 - DNA, 21 bp, Artificial Sequence
  Synthetic Construct
gaaaatctgtttgcgggacaa

SEQ ID NO: 201 - DNA, 21 bp, Artificial Sequence
  Synthetic Construct
ggtcttcagcaccacaagctt

SEQ ID NO: 202 - DNA, 21 bp, Artificial Sequence
  Synthetic Construct
ctgccgctactcactttggtt

SEQ ID NO: 203 - DNA, 22 bp, Artificial Sequence
  Synthetic Construct
tccagagcatccaatgcataag

SEQ ID NO: 204 - DNA, 21 bp, Artificial Sequence
  Synthetic Construct
tcgtcacagagaccgcgtact

SEQ ID NO: 205 - DNA, 22 bp, Artificial Sequence
  Synthetic Construct
gcttcaagatttgcctgtggtt

SEQ ID NO: 206 - DNA, 26 bp, Artificial Sequence
  Synthetic Construct
aatctcgtcatcaaacaaatcaaact

SEQ ID NO: 207 - DNA, 24 bp, Artificial Sequence
  Synthetic Construct
ttgcaaaaaaggacagaagtcaag

SEQ ID NO: 208 - DNA, 23 bp, Artificial Sequence
  Synthetic Construct
ccatggtctcattgttgatctca

SEQ ID NO: 209 - DNA, 23 bp, Artificial Sequence
  Synthetic Construct
caacaatgagaccatggatgatg

SEQ ID NO: 210 - DNA, 23 bp, Artificial Sequence
  Synthetic Construct
ctaacttgtaccgcattcaacga